SEQUENCE LISTING (145 sequences)

SEQ ID NO: 1 - protein, 160 aa, Homo sapiens
  REGION 1..160: note = Description of sequence: IL10 peptide mature form (without signal peptide)
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 178 aa, Homo sapiens
  REGION 1..178: note = Description of sequence: IL10 peptide with signal peptide.
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - protein, 160 aa, Mus musculus
  REGION 1..160: note = Description of sequence: Murine IL10 peptide mature form (without signal peptide)
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 4 - protein, 178 aa, Mus musculus
  REGION 1..178: note = Description of sequence: Murine IL10 with signal peptide
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 5 - DNA, 576 bp, Homo sapiens
  misc_feature 1..576: note = Description of sequence: Human IL-10 Wildtype
  source 1..576: mol_type = genomic DNA
  misc_feature 1..576: note = Vector Name: pExSyn2.0 - His-hIL10 WT (mp 43)
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 6 - protein, 190 aa, Homo sapiens
  REGION 1..190: note = Description of sequence: Human IL-10 Wildtype
  REGION 1..190: note = Vector Name: pExSyn2.0 - His-hIL10 WT (mp 43)
MGWSCIILFLVATATGVHSAHHHHHHHHGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 7 - DNA, 576 bp, Homo sapiens
  misc_feature 1..576: note = Description of sequence: Human IL-10 N21C
  source 1..576: mol_type = genomic DNA
  misc_feature 1..576: note = Vector Name: 078 pExSyn2.0-huIL10-8X NHis - N21C
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgccttgcatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 8 - protein, 190 aa, Homo sapiens
  REGION 1..190: note = Description of sequence: Human IL-10 N21C
  REGION 1..190: note = Vector Name: 078 pExSyn2.0-huIL10-8X NHis - N21C
MGWSCIILFLVATATGVHSAHHHHHHHHGSSPGQGTQSENSCTHFPGNLPCMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 9 - DNA, 576 bp, Homo sapiens
  misc_feature 1..576: note = Description of sequence: Human IL-10 N21D
  source 1..576: mol_type = genomic DNA
  misc_feature 1..576: note = Vector Name: 073 pExSyn2.0-huIL10-8X NHis - N21D
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctgacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 10 - protein, 190 aa, Homo sapiens
  REGION 1..190: note = Description of sequence: Human IL-10 N21D
  REGION 1..190: note = Vector Name: 073 pExSyn2.0-huIL10-8X NHis - N21D
MGWSCIILFLVATATGVHSAHHHHHHHHGSSPGQGTQSENSCTHFPGNLPDMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 11 - DNA, 576 bp, Homo sapiens
  misc_feature 1..576: note = Description of sequence: Human IL-10 N21E
  source 1..576: mol_type = genomic DNA
  misc_feature 1..576: note = Vector Name: 074 pExSyn2.0-huIL10-8X NHis - N21E
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctgagatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 12 - protein, 190 aa, Homo sapiens
  REGION 1..190: note = Description of sequence: Human IL-10 N21E
  REGION 1..190: note = Vector Name: 074 pExSyn2.0-huIL10-8X NHis - N21E
MGWSCIILFLVATATGVHSAHHHHHHHHGSSPGQGTQSENSCTHFPGNLPEMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 13 - DNA, 576 bp, Homo sapiens
  misc_feature 1..576: note = Description of sequence: Human IL-10 M22D
  source 1..576: mol_type = genomic DNA
  misc_feature 1..576: note = Vector Name: 092 pExSyn2.0-huIL10-8X NHis - M22D
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacgaccttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 14 - protein, 190 aa, Homo sapiens
  REGION 1..190: note = Description of sequence: Human IL-10 M22D
  REGION 1..190: note = Vector Name: 092 pExSyn2.0-huIL10-8X NHis - M22D
MGWSCIILFLVATATGVHSAHHHHHHHHGSSPGQGTQSENSCTHFPGNLPNDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 15 - DNA, 576 bp, Homo sapiens
  misc_feature 1..576: note = Description of sequence: Human IL-10 M22S
  source 1..576: mol_type = genomic DNA
  misc_feature 1..576: note = Vector Name: 094 pExSyn2.0-huIL10-8X NHis - M22S
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaactcgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 16 - protein, 190 aa, Homo sapiens
  REGION 1..190: note = Description of sequence: Human IL-10 M22S
  REGION 1..190: note = Vector Name: 094 pExSyn2.0-huIL10-8X NHis - M22S
MGWSCIILFLVATATGVHSAHHHHHHHHGSSPGQGTQSENSCTHFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 17 - DNA, 576 bp, Homo sapiens
  misc_feature 1..576: note = Description of sequence: Human IL-10 M22T
  source 1..576: mol_type = genomic DNA
  misc_feature 1..576: note = Vector Name: 095 pExSyn2.0-huIL10-8X NHis - M22T
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacacgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 18 - protein, 190 aa, Homo sapiens
  REGION 1..190: note = Description of sequence: Human IL-10 M22T
  REGION 1..190: note = Vector Name: 095 pExSyn2.0-huIL10-8X NHis - M22T
MGWSCIILFLVATATGVHSAHHHHHHHHGSSPGQGTQSENSCTHFPGNLPNTLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 19 - DNA, 576 bp, Homo sapiens
  misc_feature 1..576: note = Description of sequence: Human IL-10 M22W
  source 1..576: mol_type = genomic DNA
  misc_feature 1..576: note = Vector Name: 107 pExSyn2.0-huIL10-8X NHis - M22W
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaactggcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 20 - protein, 190 aa, Homo sapiens
  REGION 1..190: note = Description of sequence: Human IL-10 M22W
  REGION 1..190: note = Vector Name: 107 pExSyn2.0-huIL10-8X NHis - M22W
MGWSCIILFLVATATGVHSAHHHHHHHHGSSPGQGTQSENSCTHFPGNLPNWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 21 - DNA, 576 bp, Homo sapiens
  misc_feature 1..576: note = Description of sequence: Human IL-10 E96C
  source 1..576: mol_type = genomic DNA
  misc_feature 1..576: note = Vector Name: 154 pExSyn2.0-huIL10-8X NHis - E96C
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctggggtgcaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 22 - protein, 190 aa, Homo sapiens
  REGION 1..190: note = Description of sequence: Human IL-10 E96C
  REGION 1..190: note = Vector Name: 154 pExSyn2.0-huIL10-8X NHis - E96C
MGWSCIILFLVATATGVHSAHHHHHHHHGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGCNLKTLRLRLRRCHRFLPCENKSKAVEQVKNAFNKLQEKGIYKAMSEFDIFINYIEAYMTMKIRN

SEQ ID NO: 23 - DNA, 576 bp, Homo sapiens
  misc_feature 1..576: note = Description of sequence: Human IL-10 E96F
  source 1..576: mol_type = genomic DNA
  misc_feature 1..576: note = Vector Name: 162 pExSyn2.0-huIL10-8X NHis - E96F
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctggggttcaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 24 - protein, 190 aa, Homo sapiens
  REGION 1..190: note = Description of sequence: Human IL-10 E96F
  REGION 1..190: note = Vector Name: 162 pExSyn2.0-huIL10-8X NHis - E96F
MGWSCIILFLVATATGVHSAHHHHHHHHGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGFNLKTLRLRLRRCHRFLPCENKSKAVEQVKNAFNKLQEKGIYKAMSEFDIFINYIEAYMTMKIRN

SEQ ID NO: 25 - DNA, 576 bp, Homo sapiens
  misc_feature 1..576: note = Description of sequence: Human IL-10 E96Y
  source 1..576: mol_type = genomic DNA
  misc_feature 1..576: note = Vector Name: 163 pExSyn2.0-huIL10-8X NHis - E96Y
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctggggtacaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 26 - protein, 190 aa, Homo sapiens
  REGION 1..190: note = Description of sequence: Human IL-10 E96Y
  REGION 1..190: note = Vector Name: 163 pExSyn2.0-huIL10-8X NHis - E96Y
MGWSCIILFLVATATGVHSAHHHHHHHHGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGYNLKTLRLRLRRCHRFLPCENKSKAVEQVKNAFNKLQEKGIYKAMSEFDIFINYIEAYMTMKIRN

SEQ ID NO: 27 - DNA, 576 bp, Homo sapiens
  misc_feature 1..576: note = Description of sequence: Human IL-10 E96W
  source 1..576: mol_type = genomic DNA
  misc_feature 1..576: note = Vector Name: 164 pExSyn2.0-huIL10-8X NHis - E96W
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctggggtggaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 28 - protein, 190 aa, Homo sapiens
  REGION 1..190: note = Description of sequence: Human IL-10 E96W
  REGION 1..190: note = Vector Name: 164 pExSyn2.0-huIL10-8X NHis - E96W
MGWSCIILFLVATATGVHSAHHHHHHHHGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGWNLKTLRLRLRRCHRFLPCENKSKAVEQVKNAFNKLQEKGIYKAMSEFDIFINYIEAYMTMKIRN

SEQ ID NO: 29 - DNA, 576 bp, Homo sapiens
  misc_feature 1..576: note = Description of sequence: Human IL-10 T100C
  source 1..576: mol_type = genomic DNA
  misc_feature 1..576: note = Vector Name: 173 pExSyn2.0-huIL10-8X NHis - T100C
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagtgcctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 30 - protein, 190 aa, Homo sapiens
  REGION 1..190: note = Description of sequence: Human IL-10 T100C
  REGION 1..190: note = Vector Name: 173 pExSyn2.0-huIL10-8X NHis - T100C
MGWSCIILFLVATATGVHSAHHHHHHHHGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKCLRLRLRRCHRFLPCENKSKAVEQVKNAFNKLQEKGIYKAMSEFDIFINYIEAYMTMKIRN

SEQ ID NO: 31 - DNA, 576 bp, Homo sapiens
  misc_feature 1..576: note = Description of sequence: Human IL-10 H14D T100L
  source 1..576: mol_type = genomic DNA
  misc_feature 1..576: note = Vector Name: 469 pExSyn2.0-huIL10-8X NHis - H14D T100L
atgggatggtcctgcatcatcctgtttctggtggctaccgccaccggtgtccactctgctcaccatcaccatcaccaccatcacggatccagcccaggccagggcacccagtctgagaacagctgcaccgacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagctcctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 32 - protein, 190 aa, Homo sapiens
  REGION 1..190: note = Description of sequence: Human IL-10 H14D T100L
  REGION 1..190: note = Vector Name: 469 pExSyn2.0-huIL10-8X NHis - H14D T100L
MGWSCIILFLVATATGVHSAHHHHHHHHGSSPGQGTQSENSCTDFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRN

SEQ ID NO: 33 - DNA, 576 bp, Homo sapiens
  misc_feature 1..576: note = Description of sequence: Human IL-10 H14D N21K
  source 1..576: mol_type = genomic DNA
  misc_feature 1..576: note = Vector Name: 470 pExSyn2.0-huIL10-8X NHis - H14D N21K
atgggatggtcctgcatcatcctgtttctggtggctaccgccaccggtgtccactctgctcaccatcaccatcaccaccatcacggatccagcccaggccagggcacccagtctgagaacagctgcaccgacttcccaggcaacctgcctaagatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 34 - protein, 190 aa, Homo sapiens
  REGION 1..190: note = Description of sequence: Human IL-10 H14D N21K
  REGION 1..190: note = Vector Name: 470 pExSyn2.0-huIL10-8X NHis - H14D N21K
MGWSCIILFLVATATGVHSAHHHHHHHHGSSPGQGTQSENSCTDFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 35 - DNA, 576 bp, Homo sapiens
  misc_feature 1..576: note = Description of sequence: Human IL-10 H14D M22D
  source 1..576: mol_type = genomic DNA
  misc_feature 1..576: note = Vector Name: 471 pExSyn2.0-huIL10-8X NHis - H14D M22D
atgggatggtcctgcatcatcctgtttctggtggctaccgccaccggtgtccactctgctcaccatcaccatcaccaccatcacggatccagcccaggccagggcacccagtctgagaacagctgcaccgacttcccaggcaacctgcctaacgaccttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 36 - protein, 190 aa, Homo sapiens
  REGION 1..190: note = Description of sequence: Human IL-10 H14D M22D
  REGION 1..190: note = Vector Name: 471 pExSyn2.0-huIL10-8X NHis - H14D M22D
MGWSCIILFLVATATGVHSAHHHHHHHHGSSPGQGTQSENSCTDFPGNLPNDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 37 - DNA, 576 bp, Homo sapiens
  misc_feature 1..576: note = Description of sequence: Human IL-10 H14D M22S
  source 1..576: mol_type = genomic DNA
  misc_feature 1..576: note = Vector Name: 472 pExSyn2.0-huIL10-8X NHis - H14D M22S
atgggatggtcctgcatcatcctgtttctggtggctaccgccaccggtgtccactctgctcaccatcaccatcaccaccatcacggatccagcccaggccagggcacccagtctgagaacagctgcaccgacttcccaggcaacctgcctaactcgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 38 - protein, 190 aa, Homo sapiens
  REGION 1..190: note = Description of sequence: Human IL-10 H14D M22S
  REGION 1..190: note = Vector Name: 472 pExSyn2.0-huIL10-8X NHis - H14D M22S
MGWSCIILFLVATATGVHSAHHHHHHHHGSSPGQGTQSENSCTDFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 39 - DNA, 576 bp, Homo sapiens
  misc_feature 1..576: note = Description of sequence: Human IL-10 H14D M22A
  source 1..576: mol_type = genomic DNA
  misc_feature 1..576: note = Vector Name: 473 pExSyn2.0-huIL10-8X NHis - H14D M22A
atgggatggtcctgcatcatcctgtttctggtggctaccgccaccggtgtccactctgctcaccatcaccatcaccaccatcacggatccagcccaggccagggcacccagtctgagaacagctgcaccgacttcccaggcaacctgcctaacgcgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 40 - protein, 190 aa, Homo sapiens
  REGION 1..190: note = Description of sequence: Human IL-10 H14D M22A
  REGION 1..190: note = Vector Name: 473 pExSyn2.0-huIL10-8X NHis - H14D M22A
MGWSCIILFLVATATGVHSAHHHHHHHHGSSPGQGTQSENSCTDFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 41 - DNA, 576 bp, Homo sapiens
  misc_feature 1..576: note = Description of sequence: Human IL-10 H14D M22W
  source 1..576: mol_type = genomic DNA
  misc_feature 1..576: note = Vector Name: 474 pExSyn2.0-huIL10-8X NHis - H14D M22W
atgggatggtcctgcatcatcctgtttctggtggctaccgccaccggtgtccactctgctcaccatcaccatcaccaccatcacggatccagcccaggccagggcacccagtctgagaacagctgcaccgacttcccaggcaacctgcctaactggcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 42 - protein, 190 aa, Homo sapiens
  REGION 1..190: note = Description of sequence: Human IL-10 H14D M22W
  REGION 1..190: note = Vector Name: 474 pExSyn2.0-huIL10-8X NHis - H14D M22W
MGWSCIILFLVATATGVHSAHHHHHHHHGSSPGQGTQSENSCTDFPGNLPNWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 43 - DNA, 576 bp, Homo sapiens
  misc_feature 1..576: note = Description of sequence: Human IL-10 H14D E74K
  source 1..576: mol_type = genomic DNA
  misc_feature 1..576: note = Vector Name: 475 pExSyn2.0-huIL10-8X NHis - H14D E74K
atgggatggtcctgcatcatcctgtttctggtggctaccgccaccggtgtccactctgctcaccatcaccatcaccaccatcacggatccagcccaggccagggcacccagtctgagaacagctgcaccgacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctgaaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 44 - protein, 190 aa, Homo sapiens
  REGION 1..190: note = Description of sequence: Human IL-10 H14D E74K
  REGION 1..190: note = Vector Name: 475 pExSyn2.0-huIL10-8X NHis - H14D E74K
MGWSCIILFLVATATGVHSAHHHHHHHHGSSPGQGTQSENSCTDFPGNLPNMLRDLRDAFSRVKTFFQMKDQLDNLLLKESLLEDFKGYLGCQALSEMIQFYLKEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 45 - DNA, 576 bp, Homo sapiens
  misc_feature 1..576: note = Description of sequence: Human IL-10 H14D E96K
  source 1..576: mol_type = genomic DNA
  misc_feature 1..576: note = Vector Name: 476 pExSyn2.0-huIL10-8X NHis - H14D E96K
atgggatggtcctgcatcatcctgtttctggtggctaccgccaccggtgtccactctgctcaccatcaccatcaccaccatcacggatccagcccaggccagggcacccagtctgagaacagctgcaccgacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctggggaagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 46 - protein, 190 aa, Homo sapiens
  REGION 1..190: note = Description of sequence: Human IL-10 H14D E96K
  REGION 1..190: note = Vector Name: 476 pExSyn2.0-huIL10-8X NHis - H14D E96K
MGWSCIILFLVATATGVHSAHHHHHHHHGSSPGQGTQSENSCTDFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRN

SEQ ID NO: 47 - DNA, 576 bp, Homo sapiens
  misc_feature 1..576: note = Description of sequence: Human IL-10 H14G T100L
  source 1..576: mol_type = genomic DNA
  misc_feature 1..576: note = Vector Name: 477 pExSyn2.0-huIL10-8X NHis - H14G T100L
atgggatggtcctgcatcatcctgtttctggtggctaccgccaccggtgtccactctgctcaccatcaccatcaccaccatcacggatccagcccaggccagggcacccagtctgagaacagctgcaccggcttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagctcctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 48 - protein, 190 aa, Homo sapiens
  REGION 1..190: note = Description of sequence: Human IL-10 H14G T100L
  REGION 1..190: note = Vector Name: 477 pExSyn2.0-huIL10-8X NHis - H14G T100L
MGWSCIILFLVATATGVHSAHHHHHHHHGSSPGQGTQSENSCTGFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRN

SEQ ID NO: 49 - DNA, 576 bp, Homo sapiens
  misc_feature 1..576: note = Description of sequence: Human IL-10 H14G N21K
  source 1..576: mol_type = genomic DNA
  misc_feature 1..576: note = Vector Name: 478 pExSyn2.0-huIL10-8X NHis - H14G N21K
atgggatggtcctgcatcatcctgtttctggtggctaccgccaccggtgtccactctgctcaccatcaccatcaccaccatcacggatccagcccaggccagggcacccagtctgagaacagctgcaccggcttcccaggcaacctgcctaagatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 50 - protein, 190 aa, Homo sapiens
  REGION 1..190: note = Description of sequence: Human IL-10 H14G N21K
  REGION 1..190: note = Vector Name: 478 pExSyn2.0-huIL10-8X NHis - H14G N21K
MGWSCIILFLVATATGVHSAHHHHHHHHGSSPGQGTQSENSCTGFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 51 - DNA, 576 bp, Homo sapiens
  misc_feature 1..576: note = Description of sequence: Human IL-10 H14G M22D
  source 1..576: mol_type = genomic DNA
  misc_feature 1..576: note = Vector Name: 479 pExSyn2.0-huIL10-8X NHis - H14G M22D
atgggatggtcctgcatcatcctgtttctggtggctaccgccaccggtgtccactctgctcaccatcaccatcaccaccatcacggatccagcccaggccagggcacccagtctgagaacagctgcaccggcttcccaggcaacctgcctaacgaccttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 52 - protein, 190 aa, Homo sapiens
  REGION 1..190: note = Description of sequence: Human IL-10 H14G M22D
  REGION 1..190: note = Vector Name: 479 pExSyn2.0-huIL10-8X NHis - H14G M22D
MGWSCIILFLVATATGVHSAHHHHHHHHGSSPGQGTQSENSCTGFPGNLPNDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 53 - DNA, 576 bp, Homo sapiens
  misc_feature 1..576: note = Description of sequence: Human IL-10 H14G M22S
  source 1..576: mol_type = genomic DNA
  misc_feature 1..576: note = Vector Name: 480 pExSyn2.0-huIL10-8X NHis - H14G M22S
atgggatggtcctgcatcatcctgtttctggtggctaccgccaccggtgtccactctgctcaccatcaccatcaccaccatcacggatccagcccaggccagggcacccagtctgagaacagctgcaccggcttcccaggcaacctgcctaactcgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 54 - protein, 190 aa, Homo sapiens
  REGION 1..190: note = Description of sequence: Human IL-10 H14G M22S
  REGION 1..190: note = Vector Name: 480 pExSyn2.0-huIL10-8X NHis - H14G M22S
MGWSCIILFLVATATGVHSAHHHHHHHHGSSPGQGTQSENSCTGFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 55 - DNA, 576 bp, Homo sapiens
  misc_feature 1..576: note = Description of sequence: Human IL-10 H14G M22A
  source 1..576: mol_type = genomic DNA
  misc_feature 1..576: note = Vector Name: 481 pExSyn2.0-huIL10-8X NHis - H14G M22A
atgggatggtcctgcatcatcctgtttctggtggctaccgccaccggtgtccactctgctcaccatcaccatcaccaccatcacggatccagcccaggccagggcacccagtctgagaacagctgcaccggcttcccaggcaacctgcctaacgcgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 56 - protein, 190 aa, Homo sapiens
  REGION 1..190: note = Description of sequence: Human IL-10 H14G M22A
  REGION 1..190: note = Vector Name: 481 pExSyn2.0-huIL10-8X NHis - H14G M22A
MGWSCIILFLVATATGVHSAHHHHHHHHGSSPGQGTQSENSCTGFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 57 - DNA, 576 bp, Homo sapiens
  misc_feature 1..576: note = Description of sequence: Human IL-10 H14G M22W
  source 1..576: mol_type = genomic DNA
  misc_feature 1..576: note = Vector Name: 482 pExSyn2.0-huIL10-8X NHis - H14G M22W
atgggatggtcctgcatcatcctgtttctggtggctaccgccaccggtgtccactctgctcaccatcaccatcaccaccatcacggatccagcccaggccagggcacccagtctgagaacagctgcaccggcttcccaggcaacctgcctaactggcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 58 - protein, 190 aa, Homo sapiens
  REGION 1..190: note = Description of sequence: Human IL-10 H14G M22W
  REGION 1..190: note = Vector Name: 482 pExSyn2.0-huIL10-8X NHis - H14G M22W
MGWSCIILFLVATATGVHSAHHHHHHHHGSSPGQGTQSENSCTGFPGNLPNWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 59 - DNA, 576 bp, Homo sapiens
  misc_feature 1..576: note = Description of sequence: Human IL-10 H14G E74K
  source 1..576: mol_type = genomic DNA
  misc_feature 1..576: note = Vector Name: 483 pExSyn2.0-huIL10-8X NHis - H14G E74K
atgggatggtcctgcatcatcctgtttctggtggctaccgccaccggtgtccactctgctcaccatcaccatcaccaccatcacggatccagcccaggccagggcacccagtctgagaacagctgcaccggcttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctgaaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 60 - protein, 190 aa, Homo sapiens
  REGION 1..190: note = Description of sequence: Human IL-10 H14G E74K
  REGION 1..190: note = Vector Name: 483 pExSyn2.0-huIL10-8X NHis - H14G E74K
MGWSCIILFLVATATGVHSAHHHHHHHHGSSPGQGTQSENSCTGFPGNLPNMLRDLRDAFSRVKTFFQMKDQLDNLLLKESLLEDFKGYLGCQALSEMIQFYLKEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 61 - DNA, 576 bp, Homo sapiens
  misc_feature 1..576: note = Description of sequence: Human IL-10 H14G E96K
  source 1..576: mol_type = genomic DNA
  misc_feature 1..576: note = Vector Name: 484 pExSyn2.0-huIL10-8X NHis - H14G E96K
atgggatggtcctgcatcatcctgtttctggtggctaccgccaccggtgtccactctgctcaccatcaccatcaccaccatcacggatccagcccaggccagggcacccagtctgagaacagctgcaccggcttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctggggaagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 62 - protein, 190 aa, Homo sapiens
  REGION 1..190: note = Description of sequence: Human IL-10 H14G E96K
  REGION 1..190: note = Vector Name: 484 pExSyn2.0-huIL10-8X NHis - H14G E96K
MGWSCIILFLVATATGVHSAHHHHHHHHGSSPGQGTQSENSCTGFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRN

SEQ ID NO: 63 - DNA, 576 bp, Homo sapiens
  misc_feature 1..576: note = Description of sequence: Human IL-10 N18R
  source 1..576: mol_type = genomic DNA
  misc_feature 1..576: note = Vector Name: 279 pExSyn2.0-huIL10-8X NHis - N18R
atgggatggtcctgcatcatcctgtttctggtggctaccgccaccggtgtccactctgctcaccatcaccatcaccaccatcacggatccagcccaggccagggcacccagtctgagaacagctgcacccacttcccaggccgcctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 64 - protein, 190 aa, Homo sapiens
  REGION 1..190: note = Description of sequence: Human IL-10 N18R
  REGION 1..190: note = Vector Name: 279 pExSyn2.0-huIL10-8X NHis - N18R
MGWSCIILFLVATATGVHSAHHHHHHHHGSSPGQGTQSENSCTHFPGRLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 65 - DNA, 576 bp, Homo sapiens
  misc_feature 1..576: note = Description of sequence: Human IL-10 N18K
  source 1..576: mol_type = genomic DNA
  misc_feature 1..576: note = Vector Name: 281 pExSyn2.0-huIL10-8X NHis - N18K
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaagctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 66 - protein, 190 aa, Homo sapiens
  REGION 1..190: note = Description of sequence: Human IL-10 N18K
  REGION 1..190: note = Vector Name: 281 pExSyn2.0-huIL10-8X NHis - N18K
MGWSCIILFLVATATGVHSAHHHHHHHHGSSPGQGTQSENSCTHFPGKLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 67 - DNA, 576 bp, Homo sapiens
  misc_feature 1..576: note = Description of sequence: Human IL-10 R32N
  source 1..576: mol_type = genomic DNA
  misc_feature 1..576: note = Vector Name: 304 pExSyn2.0-huIL10-8X NHis - R32N
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagcaacgtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 68 - protein, 190 aa, Homo sapiens
  REGION 1..190: note = Description of sequence: Human IL-10 R32N
  REGION 1..190: note = Vector Name: 304 pExSyn2.0-huIL10-8X NHis - R32N
MGWSCIILFLVATATGVHSAHHHHHHHHGSSPGQGTQSENSCTHFPGNLPNMLRDLRDAFSNVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 69 - DNA, 576 bp, Homo sapiens
  misc_feature 1..576: note = Description of sequence: Human IL-10 R32Q
  source 1..576: mol_type = genomic DNA
  misc_feature 1..576: note = Vector Name: 305 pExSyn2.0-huIL10-8X NHis - R32Q
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagccaagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 70 - protein, 190 aa, Homo sapiens
  REGION 1..190: note = Description of sequence: Human IL-10 R32Q
  REGION 1..190: note = Vector Name: 305 pExSyn2.0-huIL10-8X NHis - R32Q
MGWSCIILFLVATATGVHSAHHHHHHHHGSSPGQGTQSENSCTHFPGNLPNMLRDLRDAFSQVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 71 - DNA, 576 bp, Homo sapiens
  misc_feature 1..576: note = Description of sequence: Human IL-10 R32G
  source 1..576: mol_type = genomic DNA
  misc_feature 1..576: note = Vector Name: 307 pExSyn2.0-huIL10-8X NHis - R32G
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagcggagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 72 - protein, 190 aa, Homo sapiens
  REGION 1..190: note = Description of sequence: Human IL-10 R32G
  REGION 1..190: note = Vector Name: 307 pExSyn2.0-huIL10-8X NHis - R32G
MGWSCIILFLVATATGVHSAHHHHHHHHGSSPGQGTQSENSCTHFPGNLPNMLRDLRDAFSGVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 73 - DNA, 576 bp, Homo sapiens
  misc_feature 1..576: note = Description of sequence: Human IL-10 R32C
  source 1..576: mol_type = genomic DNA
  misc_feature 1..576: note = Vector Name: 306 pExSyn2.0-huIL10-8X NHis - R32C
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagctgcgtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 74 - protein, 190 aa, Homo sapiens
  REGION 1..190: note = Description of sequence: Human IL-10 R32C
  REGION 1..190: note = Vector Name: 306 pExSyn2.0-huIL10-8X NHis - R32C
MGWSCIILFLVATATGVHSAHHHHHHHHGSSPGQGTQSENSCTHFPGNLPNMLRDLRDAFSCVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 75 - DNA, 576 bp, Homo sapiens
  misc_feature 1..576: note = Description of sequence: Human IL-10 R32P
  source 1..576: mol_type = genomic DNA
  misc_feature 1..576: note = Vector Name: 308 pExSyn2.0-huIL10-8X NHis - R32P
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagcccagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 76 - protein, 190 aa, Homo sapiens
  REGION 1..190: note = Description of sequence: Human IL-10 R32P
  REGION 1..190: note = Vector Name: 308 pExSyn2.0-huIL10-8X NHis - R32P
MGWSCIILFLVATATGVHSAHHHHHHHHGSSPGQGTQSENSCTHFPGNLPNMLRDLRDAFSPVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 77 - DNA, 576 bp, Homo sapiens
  misc_feature 1..576: note = Description of sequence: Human IL-10 R32F
  source 1..576: mol_type = genomic DNA
  misc_feature 1..576: note = Vector Name: 314 pExSyn2.0-huIL10-8X NHis - R32F
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagctttgtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 78 - protein, 190 aa, Homo sapiens
  REGION 1..190: note = Description of sequence: Human IL-10 R32F
  REGION 1..190: note = Vector Name: 314 pExSyn2.0-huIL10-8X NHis - R32F
MGWSCIILFLVATATGVHSAHHHHHHHHGSSPGQGTQSENSCTHFPGNLPNMLRDLRDAFSFVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 79 - DNA, 576 bp, Homo sapiens
  misc_feature 1..576: note = Description of sequence: Human IL-10 R32Y
  source 1..576: mol_type = genomic DNA
  misc_feature 1..576: note = Vector Name: 315 pExSyn2.0-huIL10-8X NHis - R32Y
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagctacgtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 80 - protein, 190 aa, Homo sapiens
  REGION 1..190: note = Description of sequence: Human IL-10 R32Y
  REGION 1..190: note = Vector Name: 315 pExSyn2.0-huIL10-8X NHis - R32Y
MGWSCIILFLVATATGVHSAHHHHHHHHGSSPGQGTQSENSCTHFPGNLPNMLRDLRDAFSYVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 81 - DNA, 576 bp, Homo sapiens
  misc_feature 1..576: note = Description of sequence: Human IL-10 S93G
  source 1..576: mol_type = genomic DNA
  misc_feature 1..576: note = Vector Name: 345 pExSyn2.0-huIL10-8X NHis - S93G
atgggatggtcctgcatcatcctgtttctggtggctaccgccaccggtgtccactctgctcaccatcaccatcaccaccatcacggatccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaacggcctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgataa

SEQ ID NO: 82 - protein, 190 aa, Homo sapiens
  REGION 1..190: note = Description of sequence: Human IL-10 S93G
  REGION 1..190: note = Vector Name: 345 pExSyn2.0-huIL10-8X NHis - S93G
MGWSCIILFLVATATGVHSAHHHHHHHHGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNGLGENLKTLRLRLRRCHRFLPCENKSKAVEQVKNAFNKLQEKGIYKAMSEFDIFINYIEAYMTMKIRN

SEQ ID NO: 83 - protein, 557 aa, Homo sapiens
  REGION 1..557: note = Description of sequence: the mature canonical hIL10Ra receptor subunit
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 84 - protein, 306 aa, Homo sapiens
  REGION 1..306: note = Description of sequence: mature canonical hIL10Rb receptor subunit
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 85 - protein, 330 aa, Mus musculus
  REGION 1..330: note = Description of sequence: Mature Murine IL10
MIPPPEKVRMNSVNFKNILQWEVPAFPKTNLTFTAQYESYRSFQDHCKRTASTQCDFSHLSKYGDYTVRVRAELADEHSEWVNVTFCPVEDTIIGPPEMQIESLAESLHLRFSAPQIENEPETWTLKNIYDSWAYRVQYWKNGTNEKFQVVSPYDSEVLRNLEPWTTYCIQVQGFLLDQNRTGEWSEPICERTGNDEITPSWIVAIILIVSVLVVFLFLLGCFVVLWLIYKKTKHTFRSGTSLPQHLKEFLGHPHHSTFLLFSFPPPEEAEVFDKLSIISEESEGSKQSPEDNCASEPPSDPGPRELESKDEAPSPPHDDPKLLTSTSEV

SEQ ID NO: 86 - protein, 18 aa, Homo sapiens
  REGION 1..18: note = Description of sequence: human IL10 signal peptide
MHSSALLCCLVLLTGVRA

SEQ ID NO: 87 - protein, 18 aa, Mus musculus
  REGION 1..18: note = Description of sequence: murine IL10 signal peptide
MPGSALLCCLLLLTGMRI

SEQ ID NO: 88 - protein, 160 aa, Homo sapiens; identical to SEQ ID NO: 1
  REGION 1..160: note = Description of sequence: Human IL-10 Wildtype
  REGION 1..160: note = Vector Name: pExSyn2.0 - His-hIL10 WT (mp 43)

SEQ ID NO: 89 - protein, 160 aa, Homo sapiens
  REGION 1..160: note = Description of sequence: Human IL-10 N21C
  REGION 1..160: note = Vector Name: 078 pExSyn2.0-huIL10-8X NHis - N21C
SPGQGTQSENSCTHFPGNLPCMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 90 - protein, 160 aa, Homo sapiens
  REGION 1..160: note = Description of sequence: Human IL-10 N21D
  REGION 1..160: note = Vector Name: 073 pExSyn2.0-huIL10-8X NHis - N21D
SPGQGTQSENSCTHFPGNLPDMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 91 - protein, 160 aa, Homo sapiens
  REGION 1..160: note = Description of sequence: Human IL-10 N21E
  REGION 1..160: note = Vector Name: 074 pExSyn2.0-huIL10-8X NHis - N21E
SPGQGTQSENSCTHFPGNLPEMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 92 - protein, 160 aa, Homo sapiens
  REGION 1..160: note = Description of sequence: Human IL-10 M22D
  REGION 1..160: note = Vector Name: 092 pExSyn2.0-huIL10-8X NHis - M22D
SPGQGTQSENSCTHFPGNLPNDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 93 - protein, 160 aa, Homo sapiens
  REGION 1..160: note = Description of sequence: Human IL-10 M22S
  REGION 1..160: note = Vector Name: 094 pExSyn2.0-huIL10-8X NHis - M22S
SPGQGTQSENSCTHFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 94 - protein, 160 aa, Homo sapiens
  REGION 1..160: note = Description of sequence: Human IL-10 M22T
  REGION 1..160: note = Vector Name: 095 pExSyn2.0-huIL10-8X NHis - M22T
SPGQGTQSENSCTHFPGNLPNTLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 95 - protein, 160 aa, Homo sapiens
  REGION 1..160: note = Description of sequence: Human IL-10 M22W
  REGION 1..160: note = Vector Name: 107 pExSyn2.0-huIL10-8X NHis - M22W
SPGQGTQSENSCTHFPGNLPNWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 96 - protein, 160 aa, Homo sapiens
  REGION 1..160: note = Description of sequence: Human IL-10 E96C
  REGION 1..160: note = Vector Name: 154 pExSyn2.0-huIL10-8X NHis - E96C
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGCNLKTLRLRLRRCHRFLPCENKSKAVEQVKNAFNKLQEKGIYKAMSEFDIFINYIEAYMTMKIRN

SEQ ID NO: 97 - protein, 160 aa, Homo sapiens
  REGION 1..160: note = Description of sequence: Human IL-10 E96F
  REGION 1..160: note = Vector Name: 162 pExSyn2.0-huIL10-8X NHis - E96F
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGFNLKTLRLRLRRCHRFLPCENKSKAVEQVKNAFNKLQEKGIYKAMSEFDIFINYIEAYMTMKIRN

SEQ ID NO: 98 - protein, 160 aa, Homo sapiens
  REGION 1..160: note = Description of sequence: Human IL-10 E96Y
  REGION 1..160: note = Vector Name: 163 pExSyn2.0-huIL10-8X NHis - E96Y
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGYNLKTLRLRLRRCHRFLPCENKSKAVEQVKNAFNKLQEKGIYKAMSEFDIFINYIEAYMTMKIRN

SEQ ID NO: 99 - protein, 160 aa, Homo sapiens
  REGION 1..160: note = Description of sequence: Human IL-10 E96W
  REGION 1..160: note = Vector Name: 164 pExSyn2.0-huIL10-8X NHis - E96W
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGWNLKTLRLRLRRCHRFLPCENKSKAVEQVKNAFNKLQEKGIYKAMSEFDIFINYIEAYMTMKIRN

SEQ ID NO: 100 - protein, 160 aa, Homo sapiens
  REGION 1..160: note = Description of sequence: Human IL-10 T100C
  REGION 1..160: note = Vector Name: 173 pExSyn2.0-huIL10-8X NHis - T100C
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKCLRLRLRRCHRFLPCENKSKAVEQVKNAFNKLQEKGIYKAMSEFDIFINYIEAYMTMKIRN

SEQ ID NO: 101 - protein, 160 aa, Homo sapiens
  REGION 1..160: note = Description of sequence: Human IL-10 H14D T100L
  REGION 1..160: note = Vector Name: 469 pExSyn2.0-huIL10-8X NHis - H14D T100L
SPGQGTQSENSCTDFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRN

SEQ ID NO: 102 - protein, 160 aa, Homo sapiens
  REGION 1..160: note = Description of sequence: Human IL-10 H14D N21K
  REGION 1..160: note = Vector Name: 470 pExSyn2.0-huIL10-8X NHis - H14D N21K
SPGQGTQSENSCTDFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 103 - protein, 160 aa, Homo sapiens
  REGION 1..160: note = Description of sequence: Human IL-10 H14D M22D
  REGION 1..160: note = Vector Name: 471 pExSyn2.0-huIL10-8X NHis - H14D M22D
SPGQGTQSENSCTDFPGNLPNDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 104 - protein, 160 aa, Homo sapiens
  REGION 1..160: note = Description of sequence: Human IL-10 H14D M22S
  REGION 1..160: note = Vector Name: 472 pExSyn2.0-huIL10-8X NHis - H14D M22S
SPGQGTQSENSCTDFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 105 - protein, 160 aa, Homo sapiens
  REGION 1..160: note = Description of sequence: Human IL-10 H14D M22A
  REGION 1..160: note = Vector Name: 473 pExSyn2.0-huIL10-8X NHis - H14D M22A
SPGQGTQSENSCTDFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 106 - protein, 160 aa, Homo sapiens
  REGION 1..160: note = Description of sequence: Human IL-10 H14D M22W
  REGION 1..160: note = Vector Name: 474 pExSyn2.0-huIL10-8X NHis - H14D M22W
SPGQGTQSENSCTDFPGNLPNWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 107 - protein, 160 aa, Homo sapiens
  REGION 1..160: note = Description of sequence: Human IL-10 H14D E74K
  REGION 1..160: note = Vector Name: 475 pExSyn2.0-huIL10-8X NHis - H14D E74K
SPGQGTQSENSCTDFPGNLPNMLRDLRDAFSRVKTFFQMKDQLDNLLLKESLLEDFKGYLGCQALSEMIQFYLKEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 108 - protein, 160 aa, Homo sapiens
  REGION 1..160: note = Description of sequence: Human IL-10 H14D E96K
  REGION 1..160: note = Vector Name: 476 pExSyn2.0-huIL10-8X NHis - H14D E96K
SPGQGTQSENSCTDFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRN

SEQ ID NO: 109 - protein, 160 aa, Homo sapiens
  REGION 1..160: note = Description of sequence: Human IL-10 H14G T100L
  REGION 1..160: note = Vector Name: 477 pExSyn2.0-huIL10-8X NHis - H14G T100L
SPGQGTQSENSCTGFPGNLPNMLRDLRDAFSRVKTFFQMKDQLDNLLLKESLLEDFKGYLGCQALSEMIQFYLEEVMPQAENQDPDIKAHVNSLGENLKLLRLRLRRCHRFLPCENKSKAVEQVKNAFNKLQEKGIYKAMSEFDIFINYIEAYMTMKIRN

SEQ ID NO: 110 - protein, 160 aa, Homo sapiens
  REGION 1..160: note = Description of sequence: Human IL-10 H14G N21K
  REGION 1..160: note = Vector Name: 478 pExSyn2.0-huIL10-8X NHis - H14G N21K
SPGQGTQSENSCTGFPGNLPKMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 111 - protein, 160 aa, Homo sapiens
  REGION 1..160: note = Description of sequence: Human IL-10 H14G M22D
  REGION 1..160: note = Vector Name: 479 pExSyn2.0-huIL10-8X NHis - H14G M22D
SPGQGTQSENSCTGFPGNLPNDLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 112 - protein, 160 aa, Homo sapiens
  REGION 1..160: note = Description of sequence: Human IL-10 H14G M22S
  REGION 1..160: note = Vector Name: 480 pExSyn2.0-huIL10-8X NHis - H14G M22S
SPGQGTQSENSCTGFPGNLPNSLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 113 - protein, 160 aa, Homo sapiens
  REGION 1..160: note = Description of sequence: Human IL-10 H14G M22A
  REGION 1..160: note = Vector Name: 481 pExSyn2.0-huIL10-8X NHis - H14G M22A
SPGQGTQSENSCTGFPGNLPNALRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 114 - protein, 160 aa, Homo sapiens
  REGION 1..160: note = Description of sequence: Human IL-10 H14G M22W
  REGION 1..160: note = Vector Name: 482 pExSyn2.0-huIL10-8X NHis - H14G M22W
SPGQGTQSENSCTGFPGNLPNWLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 115 - protein, 160 aa, Homo sapiens
  REGION 1..160: note = Description of sequence: Human IL-10 H14G E74K
  REGION 1..160: note = Vector Name: 483 pExSyn2.0-huIL10-8X NHis - H14G E74K
SPGQGTQSENSCTGFPGNLPNMLRDLRDAFSRVKTFFQMKDQLDNLLLKESLLEDFKGYLGCQALSEMIQFYLKEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 116 - protein, 160 aa, Homo sapiens
  REGION 1..160: note = Description of sequence: Human IL-10 H14G E96K
  REGION 1..160: note = Vector Name: 484 pExSyn2.0-huIL10-8X NHis - H14G E96K
SPGQGTQSENSCTGFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRN

SEQ ID NO: 117 - protein, 160 aa, Homo sapiens
  REGION 1..160: note = Description of sequence: Human IL-10 N18R
  REGION 1..160: note = Vector Name: 279 pExSyn2.0-huIL10-8X NHis - N18R
SPGQGTQSENSCTHFPGRLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 118 - protein, 160 aa, Homo sapiens
  REGION 1..160: note = Description of sequence: Human IL-10 N18K
  REGION 1..160: note = Vector Name: 281 pExSyn2.0-huIL10-8X NHis - N18K
SPGQGTQSENSCTHFPGKLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 119 - protein, 160 aa, Homo sapiens
  REGION 1..160: note = Description of sequence: Human IL-10 R32N
  REGION 1..160: note = Vector Name: 304 pExSyn2.0-huIL10-8X NHis - R32N
SPGQGTQSENSCTHFPGNLPNMLRDLRDAFSNVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 120 - protein, 160 aa, Homo sapiens
  REGION 1..160: note = Description of sequence: Human IL-10 R32Q
  REGION 1..160: note = Vector Name: 305 pExSyn2.0-huIL10-8X NHis - R32Q
SPGQGTQSENSCTHFPGNLPNMLRDLRDAFSQVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 121 - protein, 160 aa, Homo sapiens
  REGION 1..160: note = Description of sequence: Human IL-10 R32G
  REGION 1..160: note = Vector Name: 307 pExSyn2.0-huIL10-8X NHis - R32G
SPGQGTQSENSCTHFPGNLPNMLRDLRDAFSGVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 122 - protein, 160 aa, Homo sapiens
  REGION 1..160: note = Description of sequence: Human IL-10 R32C
  REGION 1..160: note = Vector Name: 306 pExSyn2.0-huIL10-8X NHis - R32C
SPGQGTQSENSCTHFPGNLPNMLRDLRDAFSCVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 123 - protein, 160 aa, Homo sapiens
  REGION 1..160: note = Description of sequence: Human IL-10 R32P
  REGION 1..160: note = Vector Name: 308 pExSyn2.0-huIL10-8X NHis - R32P
SPGQGTQSENSCTHFPGNLPNMLRDLRDAFSPVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 124 - protein, 160 aa, Homo sapiens
  REGION 1..160: note = Description of sequence: Human IL-10 R32F
  REGION 1..160: note = Vector Name: 314 pExSyn2.0-huIL10-8X NHis - R32F
SPGQGTQSENSCTHFPGNLPNMLRDLRDAFSFVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 125 - protein, 160 aa, Homo sapiens
  REGION 1..160: note = Description of sequence: Human IL-10 R32Y
  REGION 1..160: note = Vector Name: 315 pExSyn2.0-huIL10-8X NHis - R32Y
SPGQGTQSENSCTHFPGNLPNMLRDLRDAFSYVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 126 - protein, 160 aa, Homo sapiens
  REGION 1..160: note = Description of sequence: Human IL-10 S93G
  REGION 1..160: note = Vector Name: 345 pExSyn2.0-huIL10-8X NHis - S93G
SPGQGTQSENSCTHFPGNLPNMLRDLRDAFSRVKTFFQMKDQLDNLLLKESLLEDFKGYLGCQALSEMIQFYLEEVMPQAENQDPDIKAHVNGLGENLKTLRLRLRRCHRFLPCENKSKAVEQVKNAFNKLQEKGIYKAMSEFDIFINYIEAYMTMKIRN

SEQ ID NO: 127 - protein, 30 aa, Homo sapiens
  REGION 1..30: note = Description of sequence: terminal sequence
MGWSCIILFLVATATGVHSAHHHHHHHHGS

SEQ ID NO: 128 - DNA, 90 bp, Homo sapiens
  misc_feature 1..90: note = Description of sequence: terminal sequence
  source 1..90: mol_type = genomic DNA
atgggatggtcctgcatcatcctgtttctggtggctaccgccaccggtgtccactctgctcaccatcaccatcaccaccatcacggatcc

SEQ ID NO: 129 - protein, 4 aa, synthetic construct
  REGION 1..4: note = Description of sequence: deleted sequence
SPGQ

SEQ ID NO: 130 - protein, 5 aa, synthetic construct
  REGION 1..5: note = Description of sequence: deleted sequence
SPGQG

SEQ ID NO: 131 - protein, 6 aa, synthetic construct
  REGION 1..6: note = Description of sequence: deleted sequence
SPGQGT

SEQ ID NO: 132 - protein, 7 aa, synthetic construct
  REGION 1..7: note = Description of sequence: deleted sequence
SPGQGTQ

SEQ ID NO: 133 - protein, 8 aa, synthetic construct
  REGION 1..8: note = Description of sequence: deleted sequence
SPGQGTQS

SEQ ID NO: 134 - protein, 9 aa, synthetic construct
  REGION 1..9: note = Description of sequence: deleted sequence
SPGQGTQSE

SEQ ID NO: 135 - protein, 10 aa, synthetic construct
  REGION 1..10: note = Description of sequence: deleted sequence
SPGQGTQSEN

SEQ ID NO: 136 - protein, 20 aa, synthetic construct
  REGION 1..20: note = Description of sequence: Exemplar linker based on pattern: Gn
GGGGGGGGGGGGGGGGGGGG

SEQ ID NO: 137 - protein, 800 aa, synthetic construct
  REGION 1..800: note = Description of sequence: Exemplar linker based on pattern: GmSo
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGSSSSSSSSSSSSSSSSSSSS

SEQ ID NO: 138 - protein, 100 aa, synthetic construct
  REGION 1..100: note = Description of sequence: Exemplar linker based on pattern: GSGGSn
GSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGS

SEQ ID NO: 139 - protein, 1200 aa, synthetic construct
  REGION 1..1200: note = Description of sequence: Exemplar linker based on pattern: (GmSoGm)n
GGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGG

SEQ ID NO: 140 - protein, 2000 aa, synthetic construct
  REGION 1..2000: note = Description of sequence: Exemplar linker based on pattern: (GmSoGmSoGm)n
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGG

SEQ ID NO: 141 - protein, 480 aa, synthetic construct
  REGION 1..480: note = Description of sequence: Exemplar linker based on pattern: (GSGGSm)n
GSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSS

SEQ ID NO: 142 - protein, 480 aa, synthetic construct
  REGION 1..480: note = Description of sequence: Exemplar linker based on pattern: (GSGSmG)n
GSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSG

SEQ ID NO: 143 - protein, 460 aa, synthetic construct
  REGION 1..460: note = Description of sequence: Exemplar linker based on pattern: (GGGSm)n
GGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSS

SEQ ID NO: 144 - protein, 160 aa, synthetic construct
  REGION 1..160: note = Description of sequence: sequence pattern
  VARIANT 14: note = AA14 is H (wild-type) or C G P F or W
  VARIANT 18: note = AA18 is N (wild-type) or R or K
  VARIANT 21: note = AA21 is N (wild-type) or C D or E
  VARIANT 22: note = AA22 is M (wild-type) or D S T or W
  VARIANT 32: note = AA32 is R (wild-type) or N Q G C P F or Y
  VARIANT 93: note = AA93 is S (wild-type) or G
  VARIANT 96: note = AA96 is E (wild-type) or C F Y or W and
  VARIANT 100: note = AA100 is T (wild-type) or C
SPGQGTQSENSCTXFPGXLPXXLRDLRDAFSXVKTFFQMKDQLDNLLLKESLLEDFKGYLGCQALSEMIQFYLEEVMPQAENQDPDIKAHVNXLGXNLKXLRLRLRRCHRFLPCENKSKAVEQVKNAFNKLQEKGIYKAMSEFDIFINYIEAYMTMKIRN

SEQ ID NO: 145 - protein, 160 aa, synthetic construct
  REGION 1..160: note = Description of sequence: sequence pattern
  VARIANT 14: note = AA14 is H (wild-type) C G P F W A D E I K L M N Q R S T Y or V
  VARIANT 18: note = AA18 is N (wild-type) R K Y F A D E L V S T I V M or H.
  VARIANT 21: note = AA21 is N (wild-type) C D E A R Q H K S V I L M or T
  VARIANT 22: note = AA22 is M (wild-type) D S T W A V I L N or Q
  VARIANT 32: note = AA32 is R (wild-type) N Q G C P F Y A D E L V S T I V M or H
  VARIANT 90: note = AA90 is H (wild-type) A D E I K L M N Q R S T Y or V
  VARIANT 93: note = AA93 is S (wild-type) G E A R N D Q E I L K M or V
  VARIANT 96: note = AA96 is E (wild-type) C F Y W A N D Q H K or S
  VARIANT 100: note = AA100 is T (wild-type) C D V E A R N Q E I L K M or S
  VARIANT 104: note = AA104 is R (wild type) A W Y F H D E N Q S T I L V or M
SPGQGTQSENSCTXFPGXLPXXLRDLRDAFSXVKTFFQMKDQLDNLLLKESLLEDFKGYLGCQALSEMIQFYLEEVMPQAENQDPDIKAXVXXLGXNLKXLRLXLRRCHRFLPCENKSKAVEQVKNAFNKLQEKGIYKAMSEFDIFINYIEAYMTMKIRN

(19) United States
(12) Patent Application Publication
(10) Pub. No.: US 2025/0042966 A1
(43) Pub. Date: Feb. 6, 2025

(54) IL10 VARIANTS AND USES THEREOF

(71) Applicant: Synthekine, Inc., Menlo Park, CA (US)

(72) Inventors: Michael Wong, Menlo Park, CA (US); Scott McCauley, Menlo Park, CA (US); Andrew Morin, Menlo Park, CA (US)

(21) Appl. No.: 18/715,002

(22) PCT Filed: Dec. 1, 2022
(86) PCT No.: PCT/US2022/080769

§ 371 Date: May 30, 2024

Related U.S. Application Data
(60) Provisional application No. 63/285,019, filed on Dec. 1, 2021.

(51) Int. Cl. C07K 14/54; A61K 38/00; A61K 39/00; C12N 15/85

(52) U.S. Cl. CPC C07K 14/5428; A61K 39/4635; C12N 15/85; A61K 38/00; C12N 2800/107

(57) ABSTRACT

The present disclosure relates generally to compositions and methods for modulating signal transduction mediated by interleukin-10 (IL10). In particular, the disclosure provides IL10 polypeptide variants with altered binding affinity to interleukin-10 receptor subunit beta (IL10Rbβ). Also provided are compositions and methods useful for producing such IL10 polypeptide variants, as well as methods for modulating IL10-mediated signaling, and/or for the treatment of conditions associated with the perturbation of signal transduction mediated by IL10.

### CROSS-REFERENCES TO RELATED APPLICATIONS

This application is a 371 U.S. National Stage of PCT/US2022/080769 filed Dec. 1, 2022, which claims priority to U.S. Provisional Application No. 63/285,019, filed Dec. 1, 2021, the disclosures of which are hereby incorporated by reference in their entirety for all purposes.

### REFERENCE TO AN ELECTRONIC SEQUENCE LISTING

The contents of the electronic sequence listing (106249-1357749_ST26.xml; Size: 283,914 bytes; and Date of Creation: May 29, 2024) is herein incorporated by reference in its entirety.

### BACKGROUND OF THE INVENTION

Cytokine and growth-factor ligands typically signal through multimerization of cell surface receptors subunits. In some instances, cytokines act as multi-specific (e.g., bispecific or tri-specific) ligands which facilitate the association of the extracellular domains of receptor subunits and correspondingly bringing their intracellular domains into proximity such that intracellular signaling may occur. The nature of the cytokine ligand and its interaction with the extracellular domains of receptor subunits determines which receptor subunits are associated to form the ligand receptor complex and the intracellular signaling characteristic of such subunit combinations.

The intracellular domains of cytokine receptor subunits possess proline-rich Janus kinase (“JAK”) binding domains which are typically located in the box1/box region of the intracellular domain of the cytokine receptor subunit near the interior surface of the cell membrane. Intracellular JAK kinases associate with JAK binding domains. When the intracellular domains of cytokine receptor subunits are brought into proximity, typically by the binding of the cognate cytokine ligand for the receptor to the extracellular domains of the receptor subunits, the JAKs phosphorylate each other. Four Janus kinases have been identified in mammalian cells: JAK1, JAK2, JAK3 and TYK2. Ihle, et al. (1995) Nature 377(6550):591-4, 1995; O'Shea and Plenge (2012) Immunity 36(4):542-50. Phosphorylation of the JAK induces conformational changes in the JAK providing the ability of the JAK to further phosphorylate other intracellular proteins. The resulting phosphorylation cascade results in activation of multiple intracellular factors which transduce the intracellular signals associated with the receptor activation by the cytokine ligand. In many instances, the proteins which are phosphorylated by the JAKs are members of the signal transducer and activator of transcription (or “STAT”) protein family. Seven members of the mammalian STAT family have been identified to date: STAT1, STAT2, STAT3, STAT4, STAT5a STAT5b, and STAT6. Delgoffe, et al., (2011) Curr Opin Immunol. 23(5):632-8; Levy and Darnell (2002) Nat Rev Mol Cell Biol. 3(9):651-62 and Murray, (2007) J Immunol. 178(5):2623-9. The selective interplay of activated JAK and STAT proteins, collectively referred to as the JAK/STAT pathway, provide for a wide variety of intracellular responses observed in response to cytokine binding. Such intracellular responses initiated by the binding of a cytokine to its receptor are frequently referred to as downstream signaling.

The anti-inflammatory cytokine human interleukin-10 (hIL10), also known as human cytokine synthesis inhibitory factor (CSIF), is classified as a type(class)-2 cytokine, a group of cytokines that includes IL19, IL20, IL22, IL24 (Mda-7), and IL26, interferons (IFN-α, -β, -γ, -δ, -ε, -κ, -Ω, and -τ) and interferon-like molecules (such as limitin, IL28A, IL28B, and IL29). hIL10 is a non-covalent homodimer with a molecular mass of 37 kDa, comprised of two hIL10 monomer polypeptides. Each hIL10 monomer polypeptide is a 160 amino acid polypeptide having two intracellular disulfide bonds. Each hIL10 monomer is expressed as a proprotein comprised of 178 amino acids, the first 18 amino acids of which comprise a signal peptide. Although hIL10 is predominantly expressed by macrophages, expression has also been detected in activated T cells, B cells, mast cells, and monocytes.

Human IL10 exerts its effect through its interaction with the hIL10 receptor (hIL10R). hIL10R is a type II cytokine receptor comprising the hIL10Rα and hIL10Rβ subunits, which are also referred to as hIL10R1 and hIL10R2, respectively. Activation of the hIL10R is characterized by the binding of each hIL10 monomer to one hIL10Rα and one hIL10Rβ subunit of hIL10R, the dimeric hIL10 cytokine producing hexameric ligand/receptor hIL10R complex comprised of two hIL10 monomers, two hIL10Rα subunits and two hIL10Rβ subunits.

The hIL10Rα receptor subunit is a transmembrane protein expressed as a 578 amino acid proprotein comprising a N-terminal 21 amino acid signal sequence. The amino acid sequence of the mature canonical hIL10Rα receptor subunit is a 557 amino acid polypeptide of the sequence:

(SEQ ID NO: 83)
HGTELPSPPSVWFEAEFFHHILHWTPIPNQSESTCYEVALLRYGIESWNS
ISNCSQTLSYDLTAVTLDLYHSNGYRARVRAVDGSRHSNWTVTNTRFSVD
EVTLTVGSVNLEIHNGFILGKIQLPRPKMAPANDTYESIFSHFREYEIAI
RKVPGNFTFTHKKVKHENFSLLTSGEVGEFCVQVKPSVASRSNKGMWSKE
ECISLTRQYFTVTNVIIFFAFVLLLSGALAYCLALQLYVRRRKKLPSVLL
FKKPSPFIFISQRPSPETQDTIHPLDEEAFLKVSPELKNLDLHGSTDSGF
GSTKPSLQTEEPQFLLPDPHPQADRTLGNREPPVLGDSCSSGSSNSTDSG
ICLQEPSLSPSTGPTWEQQVGSNSRGQDDSGIDLVQNSEGRAGDTQGGSA
LGHHSPPEPEVPGEEDPAAVAFQGYLRQTRCAEEKATKTGCLEEESPLTD
GLGPKFGRCLVDEAGLHPPALAKGYLKQDPLEMTLASSGAPTGQWNQPTE
EWSLLALSSCSDLGISDWSFAHDLAPLGCVAAPGGLLGSFNSDLVTLPLI
SSLQSSE (UniProt Reference No. Q13651).

The human IL10Rβ (hIL10Rβ) receptor subunit is a transmembrane protein expressed as a 325 amino acid pro-protein comprising a 19 amino acid N-terminal signal. The amino acid sequence of the mature canonical hIL10Rb receptor subunit is a 306 amino acid polypeptide of the sequence:

(SEQ ID NO: 84)
MVPPPENVRMNSVNFKNILQWESPAFAKGNLTFTAQYLSYRIFQDKCMNT
TLTECDFSSLSKYGDHTLRVRAEFADEHSDWVNITFCPVDDTIIGPPGMQ
VEVLADSLHMRFLAPKIENEYETWTMKNVYNSWTYNVQYWKNGTDEKFQI
TPQYDFEVLRNLEPWTTYCVQVRGFLPDRNKAGEWSEPVCEQTTHDETVP
SWMVAVILMASVFMVCLALLGCFALLWCVYKKTKYAFSPRNSLPQHLKEF
LGHPHHNTLLFFSFPLSDENDVFDKLSVIAEDSESGKQNPGDSCSLGTPP
GQGPQS (UniProt Reference No. Q08334).

Amino acids 20-220 (amino acids 1-201 of the mature hIL10Rβ protein) correspond to the extracellular domain, amino acids 221-242 (amino acids 202-223 of the mature hIL10Rβ protein) correspond to the 22 amino acid transmembrane domain, and amino acids 243-325 (amino acids 224-306 of the mature hIL10Rβ protein) correspond to the intracellular domain.

The murine IL10Rβ (mIL10Rβ) receptor subunit is expressed as a 349 amino acid pro-protein comprising a 19 amino acid N-terminal signal sequence. The amino acid sequence of the mature canonical hIL10Rb receptor subunit is a 330 amino acid polypeptide of the sequence:

(SEQ ID NO: 85)
MIPPPEKVRMNSVNFKNILQWEVPAFPKTNLTFTAQYESYRSFQDHCKRT
ASTQCDFSHLSKYGDYTVRVRAELADEHSEWVNVTFCPVEDTIIGPPEMQ
IESLAESLHLRFSAPQIENEPETWTLKNIYDSWAYRVQYWKNGTNEKFQV
VSPYDSEVLRNLEPWTTYCIQVQGFLLDQNRTGEWSEPICERTGNDEITP
SWIVAIILIVSVLVVFLFLLGCFVVLWLIYKKTKHTFRSGTSLPQHLKEF
LGHPHHSTELLFSFPPPEEAEVFDKLSIISEESEGSKQSPEDNCASEPPS
DPGPRELESKDEAPSPPHDDPKLLTSTSEV

mIL2Rβ is referenced at UniProtKB database as entry Q61190. Amino acids 20-220 (amino acids 1-201 of the mature protein) correspond to the extracellular domain, amino acids 221-241 (amino acids 202-222 of the mature protein) correspond to the 21 amino acid transmembrane domain, and amino acids 242-349 (amino acids 223-330 of the mature protein) correspond to the intracellular domain.

The interaction of IL10 with its receptor and receptor subunits have been studied and described in the scientific literature, Pletnev, et al. provide information relating to the structures of the soluble receptor chain of IL10R2 and the ternary complex of IL10/sIL10R1/sIL10R2 and residues involved in ligand-receptor and receptor-receptor interactions. Pletnev, et al. (2005) BMC Structural Biology 5:10. Although the interaction between hIL10 and the hIL10Rα receptor subunit is a specific high-affinity interaction, the association of hIL10 with hIL10Rβ is a comparatively low affinity interaction. Reports suggest that the interaction of hIL10 with hIL10Rα induces a conformational change which in hIL10 and/or hIL10Rα which facilitates the binding of the [hIL10:hIL10Rα] complex to hIL10Rβ. The formation of the ternary [hIL10:hIL10Rα:hIL10Rβ] complex is suggested as the limiting factor in initiating hIL10 signaling.

The interaction of IL-10 with the IL10R effects the activation of JAK1 (associated with hIL10Rα) and Tyk2 (associated with hIL10Rβ) and induces the activation of STAT1, STAT3, and, in some cells, STAT5. STAT3 is recruited directly to the hIL-10/hIL-10R complex via either of two tyrosine residues in the hIL10Rα cytoplasmic domain that become phosphorylated in response to hIL-10 and are required for hIL-10 signaling. Homodimerization of STAT3 results in its release from the receptor and translocation of the phosphorylated STAT homodimer into the nucleus, where it binds to STAT3-binding elements in the promoters of numerous genes, including the promoter of IL10, which is positively regulated by STAT3. The hIL10 receptor intracellular domain possesses particular sequences that are linked to its anti-inflammatory activity that are not shared by other STAT3 activating cytokine receptors. Riley, et al (1999) Journal of Biological Chemistry 274(23):15967-16664.

The expression of the hIL10Rα ad IL10Rb receptor subunits vary with respect to cell type and the activation state of the cell. The hIL10Rα receptor subunit is a “private” or “proprietary” subunit exclusive to the hIL10 receptor. In contrast, the hIL10Rβ subunit is shared with other cytokine receptors including IL22, IL26, IL28, and the interferon lambda L1 (IFNλ1) receptor complexes. The activation state of cells that express hIL10Rα may result in substantial variability in the levels of expression. The expression hIL10Rα in hematopoietic cells, which constitutively express low levels of hIL10Rα, is frequently substantially upregulated by various stimuli. In contrast to hIL10Rα, which is expressed primarily on hematopoietic cells, the IL10Rb receptor subunit is expressed ubiquitously. While certain cell types express hIL10Rβ at different levels, the level of hIL10Rβ expression in a given cell type is typically less affected by the activation state of the cell than hIL10Rα.

hIL10 is associated with a wide variety of functions and exhibits both immunosuppressive and immunostimulatory activities through its interaction with T cells, B cells, macrophages, and antigen presenting cells (APCs). The immunosuppressive activity of hIL10 is well documented. hIL10 is associated with suppressing the expression of IL1α, IL1β, IL6, IL8, TNFα, GM-CSF and G-CSF in activated monocytes and activated macrophages, as well as suppression of proinflammatory cytokine interferon-gamma (INFγ) production by NK cells. However, hIL10 also demonstrates an immunostimulatory effect by stimulation of the production of the proinflammatory cytokine IFN-γ by CD8+ T cells. The immunostimulatory and immunosuppressive properties of hIL10 have proved to be a challenge in the clinical application of hIL10 in the treatment of human disease.

Saxton, et al. describe the interaction of IL10 with the IL10Rb subunit and amino acid residues that are involved in the binding of IL10 to IL10Rβ and that modification of such residues can potentially provide IL10 variants that retain the immunosuppressive function of IL10 on myeloid cells but have diminished pro-inflammatory activity on CD8+ T cells. Saxton, et al. (2021) Science 371:eabc8433.

### SUMMARY OF THE DISCLOSURE

The present disclosure provides compositions that are useful for modulating signal transduction mediated by interleukin-10 (IL10). In particular, the disclosure provides IL10 muteins comprising at least one IL10 monomer variant, the monomer variant comprising one or more amino acid substitutions. Also provided are compositions and methods useful for producing such IL10 monomer variants, as well as methods for modulating IL10-mediated signaling, and for the treatment of conditions associated with the perturbation of signal transduction mediated by IL10.

In one aspect, present disclosure provides an hIL10 monomer variant having at least 70% sequence identity (e.g., at least 70%, 75%, 80%, 85%, 90%, 91%, 92%, 93%, 94%, 95%, 96%, 97%, 98%, or 99% sequence identity) to SEQ ID NO:1 and comprising one or more amino acid substitutions at a position corresponding to residues H14, N18, N21, M22, D25, R32, S93, E96, and T100 of SEQ ID NO:1 (mature human IL10 peptide). In other words, in one aspect, the present disclosure provides an hIL10 monomer variant comprising one or more amino acid substitutions at a position corresponding to residues H14, N18, N21, M22, D25, R32, S93, E96, and T100 of SEQ ID NO:1, wherein the amino acid sequence of the hIL10 monomer variant has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:1. In some embodiments, present disclosure provides an hIL10 monomer variant comprising one or more amino substitutions at the amino acid residues selected from the group consisting of H14, N18, N21, M22, D25, R32, S93, E96, and T100 of SEQ ID NO:1.

In some embodiments, the amino acid substitution at position H14 is selected from the group consisting of H14C, H14F, H14P, H14W and H14G.

In some embodiments, the amino acid substitution at position N18 is selected from the group consisting of N18R and N18K.

In some embodiments, the amino acid substitution at position N21 is selected from the group consisting of N21C, N21D and N21E.

In some embodiments, the amino acid substitution at position M22 is selected from the group consisting of M22D, M22S, M22T, and M22W.

In some embodiments, the amino acid substitution at position D25 is selected from the group consisting of D25P and D25Q.

In some embodiments, the amino acid substitution at position R32 is selected from the group consisting of R32N, R32Q, R32G, R32C, R32P, R32F and R32Y.

In some embodiments, the amino acid substitution at position S93 is S93G.

In some embodiments, the amino acid substitution at position E96 is selected from the group consisting of E96C, E96F, E96Y, and E96W.

In some embodiments, the amino acid substitution at position T100 is T100C.

In some embodiments, present disclosure provides an hIL10 monomer variant having at least 70% sequence identity (e.g., at least 70%, 75%, 80%, 85%, 90%, 91%, 92%, 93%, 94%, 95%, 96%, 97%, 98%, or 99% sequence identity) to SEQ ID NO:1, the hIL10 monomer variant comprising one or more amino acid substitutions selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W and T100C numbered in accordance with SEQ ID NO:1. In some embodiments, the present disclosure provides an hIL10 monomer variant comprising one or more amino acid substitutions selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W and T100C numbered in accordance with SEQ ID NO: 1, wherein the amino acid sequence of the hIL10 monomer variant has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:1. In some embodiments, present disclosure provides an hIL10 monomer variant comprising one or more amino acid substitutions selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W and T100C numbered in accordance with SEQ ID NO:1.

In some embodiments, the present disclosure provides an hIL10 monomer variant having at least 70% sequence identity (e.g., at least 70%, 75%, 80%, 85%, 90%, 91%, 92%, 93%, 94%, 95%, 96%, 97%, 98%, 99% or 100% sequence identity) to an amino acid sequence in Table 1. In some embodiments, the hIL10 monomer variant is encoded by a nucleic acid sequence in Table 1. In some embodiments, the hIL10 monomer variant comprises a sequence having at least 70% sequence identity (e.g., at least 70%, 75%, 80%, 85%, 90%, 91%, 92%, 93%, 94%, 95%, 96%, 97%, 98%, 99% or 100% sequence identity) to an amino acid sequence in Table 1 without the amino terminal sequence MGWSCIILFLVATATGVHSAHHHHHHHHGS. In some embodiments, the hIL10 monomer variant is encoded by a nucleic acid sequence in Table 1 without a 5′ nucleic acid sequence encoding the amino acid sequence MGWSCIILFLVATATGVHSAHHHHHHHHGS (e.g., without the nucleic acid sequence ATGGGATGGTCCTGCATCATCCTGTTTCTGGTGGCTACCGCCACCGGTGTCCACT CTGCTCACCATCACCATCACCACCATCACGGATCC).

In some embodiments, the present disclosure provides an hIL10 monomer variant having at least 70% sequence identity (e.g., at least 70%, 75%, 80%, 85%, 90%, 91%, 92%, 93%, 94%, 95%, 96%, 97%, 98%, 99% or 100% sequence identity) to an amino acid sequence in Table 2. In some embodiments, the present disclosure provides an hIL10 monomer variant having at least 70% sequence identity (e.g., at least 70%, 75%, 80%, 85%, 90%, 91%, 92%, 93%, 94%, 95%, 96%, 97%, 98%, 99% or 100% sequence identity) to an amino acid sequence selected from SEQ ID NO:88 to SEQ ID NO:126 (e.g., SEQ ID NOs:88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or 126). In some embodiments, the present disclosure provides an hIL10 monomer variant comprising an amino acid sequence selected from SEQ ID NO:88 to SEQ ID NO:126 (e.g., SEQ ID NOs:88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or 126).

In some embodiments, present disclosure provides an hIL10 monomer variant having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1), and the hIL10 monomer variant comprises one or more amino acid substitutions selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C numbered in accordance with SEQ ID NO:1, optionally further comprising one or more additional amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO:1. In some embodiments, present disclosure provides an hIL10 monomer variant comprising one or more amino acid substitutions selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C numbered in accordance with SEQ ID NO:1, and optionally further comprising one or more additional amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO:1

In some embodiments, present disclosure provides an hIL10 monomer variant comprising one or more amino acid substitutions selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C numbered in accordance with SEQ ID NO:1, optionally further comprising one or more additional amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO:1, wherein the amino acid sequence of the hIL10 monomer variant has at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1).

In some embodiments, the present disclosure provides homodimeric or heterodimeric hIL10 muteins comprised of at least one hIL10 monomer variant, the hIL10 monomer variant having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1), and the hIL10 monomer variant comprises one or more amino acid substitutions at a position corresponding to residues H14, N18, N21, M22, D25, R32, S93, E96, and T100 of SEQ ID NO:1. In some embodiments, the present disclosure provides homodimeric or heterodimeric hIL10 muteins comprised of at least one hIL10 monomer variant comprising one or more amino acid substitutions at a position corresponding to residues H14, N18, N21, M22, D25, R32, S93, E96, and T100 of SEQ ID NO:1, wherein the amino acid sequence of the hIL10 monomer variant has at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1). In some embodiments, the present disclosure provides homodimeric or heterodimeric hIL10 muteins comprised of at least one hIL10 monomer variant, the hIL10 monomer variant comprising one or more amino acid substitutions at the amino acid residues selected from the group consisting of H14, N18, N21, M22, D25, R32, S93, E96, and T100 of SEQ ID NO:1.

In some embodiments, the present disclosure provides homodimeric or heterodimeric hIL10 muteins comprised of at least one hIL10 monomer variant, the hIL10 monomer variant having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO: 1), comprising one or more amino acid substitutions selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C numbered in accordance with SEQ ID NO: 1, optionally further comprising one or more additional amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO:1.

In some embodiments, the present disclosure provides homodimeric or heterodimeric hIL10 muteins comprised of at least one hIL10 monomer variant comprising one or more amino acid substitutions selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C numbered in accordance with SEQ ID NO:1, optionally further comprising one or more additional amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO:1, wherein the amino acid sequence of the hIL10 monomer variant has at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1).

In some embodiments, the hIL10 mutein is provided in a homodimeric form comprised of two hIL10 variant polypeptide monomers each having the same amino acid sequence. In other embodiments, the present disclosure provides heterodimeric hIL10 muteins wherein the hIL10 monomers comprising the hIL10 mutein have different amino acid sequences. In one aspect, the present disclosure provides a heterodimeric hIL10 mutein comprised of: (a) an hIL10 variant polypeptide monomer having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO: 1) and comprising one or more amino acid substitutions at positions corresponding to residues H14, N18, N21, M22, D25, R32, S93, E96, and T100 of SEQ ID NO:1, and (b) a wild-type hIL10 monomer. In one aspect, the present disclosure provides a heterodimeric hIL10 mutein comprised of: (a) an hIL10 variant polypeptide monomer comprising one or more amino acid substitutions at positions corresponding to residues H14, N18, N21, M22, D25, R32, S93, E96, and T100 of SEQ ID NO:1, wherein the amino acid sequence of the hIL10 variant polypeptide monomer has at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1), and (b) a wild-type hIL10 monomer. In one embodiment, the present disclosure provides a heterodimeric hIL10 mutein comprised of: (a) an hIL10 variant polypeptide monomer comprising one or more amino acid substitutions at positions corresponding to residues H14, N18, N21, M22, D25, R32, S93, E96, and T100 of SEQ ID NO:1, and (b) a wild-type hIL10 monomer.

In another aspect, the present disclosure provides a heterodimeric hIL10 mutein comprised of: (a) a first hIL10 variant polypeptide monomer having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO: 1) and comprising one or more amino acid substitutions at positions corresponding to residues H14, N18, N21, M22, R32, H90, N92, S93, E96, K99 and T100 of SEQ ID NO:1; and (b) a second hIL10 variant polypeptide monomer having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO: 1) and comprising one or more amino acid substitutions at positions corresponding to residues H14, N18, N21, M22, R32, H90, N92, S93, E96, K99 and T100 of SEQ ID NO:1; wherein the first hIL10 variant polypeptide monomer and second hIL10 variant polypeptide monomer are the same (i.e., both the first and second hIL10 variant polypeptide monomers have the same amino acid sequence). In one aspect, the present disclosure provides a heterodimeric hIL10 mutein comprised of: (a) a first hIL10 variant polypeptide monomer comprising one or more amino acid substitutions at positions corresponding to residues H14, N18, N21, M22, R32, H90, N92, S93, E96, K99 and T100 of SEQ ID NO:1, wherein the amino acid sequence of the hIL10 variant polypeptide monomer has at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1); and (b) a second hIL10 variant polypeptide monomer comprising one or more amino acid substitutions at positions corresponding to residues H14, N18, N21, M22, R32, H90, N92, S93, E96, K99 and T100 of SEQ ID NO:1, wherein the amino acid sequence of the hIL10 variant polypeptide monomer has at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1); wherein the first hIL10 variant polypeptide monomer and second hIL10 variant polypeptide monomer are the same (i.e., both the first and second hIL10 variant polypeptide monomers have the same amino acid sequence).

In another aspect, the present disclosure provides a heterodimeric hIL10 mutein comprised of: (a) a first hIL10 variant polypeptide monomer having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO: 1) and comprising one or more amino acid substitutions at positions corresponding to residues H14, N18, N21, M22, R32, H90, N92, S93, E96, K99 and T100 of SEQ ID NO:1; and (b) a second hIL10 variant polypeptide monomer having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO: 1) and comprising one or more amino acid substitutions at positions corresponding to residues H14, N18, N21, M22, R32, H90, N92, S93, E96, K99 and T100 of SEQ ID NO:1; wherein the first hIL10 variant polypeptide monomer and second hIL10 variant polypeptide monomer are different (i.e., the first and second hIL10 variant polypeptide monomers have different amino acid sequences). In one aspect, the present disclosure provides a heterodimeric hIL10 mutein comprised of: (a) a first hIL10 variant polypeptide monomer comprising one or more amino acid substitutions at positions corresponding to residues H14, N18, N21, M22, R32, H90, N92, S93, E96, K99 and T100 of SEQ ID NO:1, wherein the amino acid sequence of the hIL10 variant polypeptide monomer has at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1); and (b) a second hIL10 variant polypeptide monomer comprising one or more amino acid substitutions at positions corresponding to residues H14, N18, N21, M22, R32, H90, N92, S93, E96, K99 and T100 of SEQ ID NO:1, wherein the amino acid sequence of the hIL10 variant polypeptide monomer has an amino acid sequence having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO: 1); wherein the first hIL10 variant polypeptide monomer and second hIL10 variant polypeptide monomer are different (i.e., the first and second hIL10 variant polypeptide monomers have different amino acid sequences).

In some embodiments, the present disclosure provides an hIL10 monomer variant having at least 70% sequence identity (e.g., at least 70%, 75%, 80%, 85%, 90%, 91%, 92%, 93%, 94%, 95%, 96%, 97%, 98%, or 99% sequence identity) to a sequence comprising a deletion of one to 10 contiguous amino acids (e.g., a deletion of 1, 2, 3, 4, 5, 6, 7, 8, 9 or 10 contiguous amino acids) from the amino terminus of SEQ ID NO:1. In some embodiments, the present disclosure provides an hIL10 monomer variant having at least 70% sequence identity (e.g., at least 70%, 75%, 80%, 85%, 90%, 91%, 92%, 93%, 94%, 95%, 96%, 97%, 98%, or 99% sequence identity) to a sequence comprising a deletion of one to 10 contiguous amino acids (e.g., a deletion of 1, 2, 3, 4, 5, 6, 7, 8, 9 or 10 contiguous amino acids) from the amino acid sequence SPGQGTQSEN located at the amino terminus of SEQ ID NO:1 (e.g., a deletion of S, SP, SPG, SPGQ, SPGQG, SPGQGT, SPGQGTQ, SPGQGTQS, SPGQGTQSE, or SPGQGTQSEN from the amino terminus of SEQ ID NO:1).

In some embodiments, the hIL10 monomer variant having at least 70% sequence identity (e.g., at least 70%, 75%, 80%, 85%, 90%, 91%, 92%, 93%, 94%, 95%, 96%, 97%, 98%, or 99% sequence identity) to a sequence comprising an amino terminal deletion of one to 10 contiguous amino acids (e.g., a deletion of 1, 2, 3, 4, 5, 6, 7, 8, 9 or 10 contiguous amino acids) from the amino terminus of SEQ ID NO:1 described herein comprises one or more amino acid substitutions at a position corresponding to residues H14, N18, N21, M22, D25, R32, S93, E96, and T100 of SEQ ID NO:1. In some embodiments, the hIL10 monomer variant having at least 70% sequence identity (e.g., at least 70%, 75%, 80%, 85%, 90%, 91%, 92%, 93%, 94%, 95%, 96%, 97%, 98%, or 99% sequence identity) to a sequence comprising an amino terminal deletion of one to 10 contiguous amino acids (e.g., a deletion of 1, 2, 3, 4, 5, 6, 7, 8, 9 or 10 contiguous amino acids) from the amino terminus of SEQ ID NO:1 described herein comprises one or more amino acid substitutions selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C numbered in accordance with SEQ ID NO:1. In some embodiments, the hIL10 monomer variant having at least 70% sequence identity (e.g., at least 70%, 75%, 80%, 85%, 90%, 91%, 92%, 93%, 94%, 95%, 96%, 97%, 98%, or 99% sequence identity) to a sequence comprising an amino terminal deletion of one to 10 amino acids (e.g., a deletion of 1, 2, 3, 4, 5, 6, 7, 8, 9 or 10 amino acids) from the amino terminus of SEQ ID NO:1 described herein further comprises one or more additional amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO:1.

In some embodiments, the present disclosure provides homodimeric or heterodimeric hIL10 muteins comprised of at least one hIL10 monomer variant, the hIL10 monomer variant having at least 70% sequence identity (e.g., at least 70%, 75%, 80%, 85%, 90%, 91%, 92%, 93%, 94%, 95%, 96%, 97%, 98%, or 99% sequence identity to SEQ ID NO:1) to a sequence comprising a deletion of one to 10 contiguous amino acids (e.g., a deletion of 1, 2, 3, 4, 5, 6, 7, 8, 9 or 10 contiguous amino acids) from the amino terminus of SEQ ID NO:1. In some embodiments, the present disclosure provides homodimeric or heterodimeric hIL10 muteins comprised of at least one hIL10 monomer variant, the hIL10 monomer variant having at least 70% sequence identity (e.g., at least 70%, 75%, 80%, 85%, 90%, 91%, 92%, 93%, 94%, 95%, 96%, 97%, 98%, or 99% sequence identity to SEQ ID NO:1) to a sequence comprising a deletion of one to 10 contiguous amino acids (e.g., a deletion of 1, 2, 3, 4, 5, 6, 7, 8, 9 or 10 contiguous amino acids) from the amino acid sequence SPGQGTQSEN located at the amino terminus of SEQ ID NO:1 (e.g., a deletion of S, SP, SPG, SPGQ, SPGQG, SPGQGT, SPGQGTQ, SPGQGTQS, SPGQGTQSE, or SPGQGTQSEN from the amino terminus of SEQ ID NO:1).

In some embodiments, the homodimeric or heterodimeric hIL10 muteins comprised of at least one hIL10 monomer variant having at least 70% sequence identity (e.g., at least 70%, 75%, 80%, 85%, 90%, 91%, 92%, 93%, 94%, 95%, 96%, 97%, 98%, or 99% sequence identity to SEQ ID NO: 1) to a sequence comprising a deletion of one to 10 contiguous amino acids (e.g., a deletion of 1, 2, 3, 4, 5, 6, 7, 8, 9 or 10 contiguous amino acids, or a deletion of S, SP, SPG, SPGQ, SPGQG, SPGQGT, SPGQGTQ, SPGQGTQS, SPGQGTQSE, or SPGQGTQSEN) from the amino terminus of SEQ ID NO:1 described herein comprise: (i) one or more amino acid substitutions at a position corresponding to residues H14, N18, N21, M22, D25, R32, S93, E96, and T100 of SEQ ID NO:1; (ii) one or more amino acid substitutions selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C numbered in accordance with SEQ ID NO:1; or (iii) further comprise one or more additional amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO:1.

In some embodiments, the hIL10 muteins of the present disclosure provide greater activity on cells which have higher surface expression of the hIL10Rβ subunit (e.g., cells of myeloid origin) relative to cells with that have lower surface expression of the hIL10Rβ receptor subunit. Cell types which are characterized as having high surface expression of the hIL10Rβ subunit include cells of myeloid origin. Cell types which are characterized as having low surface expression of the hIL10R2 receptor subunit include lymphocytes such as CD8+ T cells, CD4+ T cells, B cells or NK cells.

In some embodiments, the present disclosure provides a hIL10 muteins that exhibit a prolonged duration of action in vivo in a mammalian subject and pharmaceutically acceptable formulations thereof. In some embodiments, such hIL10 muteins having prolonged duration of action in vivo are achieved by conjugation of one or both of the hIL10 polypeptide monomers of the homodimeric or heterodimeric hIL10 mutein to one or more carrier molecules. In some embodiments, the carrier molecule is a protein carrier molecule such as human serum albumin which may be provided as a fusion protein with one or both hIL10 polypeptide monomers, optionally comprising a polypeptide linker sequence between the hIL10 monomer and human serum albumin sequence.

In some embodiments, when the hIL10 variant is provided as an Fc fusion, the hIL10 variant comprises: (a) a fusion protein comprising a first hIL10 polypeptide monomer and a first Fc monomer, optionally comprising a first linker molecule between the first IL10 monomer sequence and the first Fc monomer sequence, and (b) a fusion protein comprising a second hIL10 polypeptide monomer and a second Fc monomer, optionally comprising a second linker molecule between the second IL10 monomer sequence and the second Fc monomer sequence, wherein one or more of the hIL10 polypeptide monomers is an hIL10 monomer variant comprising one or more amino substitutions corresponding to amino acid residues selected from the group consisting of H14, N18, N21, M22, R32, H90, N92, S93, E96, K99 and T100 of SEQ ID NO: 1. In some embodiments, the first and second Fc monomers are modified to promote heterodimerization (e.g. a “knob-into-hole”) which are particularly useful when the first and second hIL10 monomers comprising the hIL10 variant are different (e.g. heterodimeric hIL10/Fc mutein) so as to maintain a 1:1 ratio of the differing hIL10 monomers. In some embodiments, the Fc monomers are modified to reduce effector function, eliminate glycosylation sites, extend duration of action, eliminate unpaired cysteine residue, and combinations thereof.

In some embodiments, such hIL10 muteins having prolonged duration of action in vivo are achieved by conjugation of one or both of the hIL10 polypeptide monomers of the hIL10 variant to a water-soluble polymeric carrier. In some embodiments, the water-soluble polymeric carrier is polyethylene glycol (PEG). In some embodiments, the PEG is a linear or branched PEG molecule having molecular weights from about 2,000 to about 80,000 daltons, alternatively about 5,000 daltons to about 80,000 daltons alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms. In some embodiments, the PEG is conjugated to the N-terminus of the hIL10 mutein. In some embodiments, the PEG is conjugated to the N-terminus of one or both hIL10 monomer polypeptides of the hIL10 mutein. In some embodiments, the carrier molecule is conjugated to the hIL10 mutein via a linker. In some embodiments, the carrier molecule is conjugated to one or both hIL10 monomers of the hIL10 mutein via a linker. In some embodiments, the present disclosure provides a composition comprising a mixture of a “monoPEGylated” hIL10 mutein (i.e., an hIL10 mutein wherein only one hIL10 monomer of the hIL10 mutein is PEGylated) and a “diPEGylated” hIL10 mutein (i.e., an hIL10 mutein wherein both hIL10 monomers of the hIL10 mutein are PEGylated). In some embodiments, the ratio of the monoPEGylated hIL10 mutein and the diPEGylated hIL10 mutein species in such composition are approximately 1:1. In some embodiments, the present disclosure provides a hIL10 mutein composition comprising a mixture of a nonPEGylated hIL10 mutein, a monopegylated hIL10 mutein and a diPEGylated hIL10 mutein and one or more pharmaceutically acceptable carriers.

In another aspect, the disclosure provides a nucleic acid molecule comprising a nucleic acid sequence encoding a hIL10 monomer variant or mutein disclosed herein. In some embodiments, the nucleic acid sequence further encodes a signal peptide. In some embodiments, the nucleic acid sequence further encodes a peptide linker. In some embodiments, the nucleic acid sequence is operably linked to one or more heterologous nucleic acid sequences. In some embodiments, the heterologous nucleic acid sequence is an expression control sequence. In some embodiments, the expression control sequence is functional in a mammalian cell.

In another aspect, the disclosure also provides a vector comprising a nucleic acid sequence encoding a hIL10 monomer variant or mutein disclosed herein. In some embodiments, the vector is an expression vector. In some embodiments, the vector is viral vector. In some embodiments, the vector is non-viral vector.

In another aspect, the disclosure provides a recombinantly modified cell comprising a nucleic acid molecule or vector of the disclosure.

In another aspect, the disclosure provides a cell culture comprising at least one recombinantly modified cell of the disclosure, and a culture medium.

The present disclosure further provides methods for the recombinant production, isolation, purification and characterization of a hIL10 mutein described herein. Thus, in another aspect, the disclosure provides a method for producing a hIL10 mutein of the disclosure. In some embodiments, the method comprises a) providing one or more recombinantly modified cells comprising a nucleic acid molecule or vector comprising a nucleic acid sequence encoding a hIL10 mutein disclosed herein; and b) culturing the one or more cells in a culture medium such that the cells produce the hIL10 mutein encoded by the nucleic acid sequence. In some embodiments, the method further comprises the step of (c) isolating and/or purifying the hIL10 mutein.

In another aspect, the disclosure provides a hIL10 mutein produced by the above method.

In another aspect, the disclosure provides a pharmaceutical composition. In some embodiments, the pharmaceutical composition comprises a hIL10 mutein monomer or dimer of the disclosure. In some embodiments, the pharmaceutical composition comprises a pharmaceutically acceptable carrier.

In some embodiments, the present disclosure provides a pharmaceutical composition comprising a mixture of a “monoPEGylated” hIL10 mutein (i.e., an hIL10 mutein wherein only one hIL10 monomer of the hIL10 mutein is PEGylated) and a “diPEGylated” hIL10 mutein (i.e., an hIL10 mutein wherein both hIL10 monomers of the hIL10 mutein are PEGylated) and one or more pharmaceutically acceptable carriers. In some embodiments, the ratio of the monoPEGylated hIL10 mutein and the diPEGylated hIL10 mutein species in such pharmaceutical formulation are approximately 1:1. In some embodiments, the present disclosure provides a pharmaceutical formulation comprising a mixture of a nonPEGylated hIL10 mutein, a monopegylated hIL10 mutein and a diPEGylated hIL10 mutein and one or more pharmaceutically acceptable carriers. In some embodiments, the pharmaceutical composition comprises a nucleic acid molecule or vector of the disclosure. In some embodiments, the pharmaceutical composition comprises a recombinantly modified cell of the disclosure. In some embodiments, the recombinantly modified cell is a mammalian cell.

In another aspect, the disclosure provides a method for modulating IL10-mediated signaling in a subject, the method comprising administering to the subject an effective amount of a pharmaceutical composition described herein. In some embodiments, the IL10-mediated signaling comprises STAT3-mediated signaling. In some embodiments, the STAT3-mediated signaling is determined by an assay selected from the group consisting of a gene expression assay, a phospho-flow signaling assay, and an enzyme-linked immunosorbent assay (ELISA). In some embodiments, the STAT3-mediated signaling in the subject is reduced by about 20% to about 100% compared to a reference level. In some embodiments, the administered composition results in a reduced capacity to induce expression of a pro-inflammatory gene selected from IFN-γ, granzyme B, granzyme A, perform, TNF-α, GM-CSF, and MIP1α in the subject.

The hIL10 variants of the present disclosure are useful in the treatment or prevention of disease in mammalian subjects. Thus, in another aspect, the disclosure provides a method for treating a health condition in a subject in need thereof, the method comprising administering to the subject a therapeutically effective amount of composition comprising: a hIL10 variant or hIL10 mutein described herein; a nucleic acid molecule or vector comprising a nucleic acid sequence encoding a hIL10 variant or hIL10 mutein described herein; a recombinantly modified cell comprising a nucleic acid molecule or vector described herein; or a pharmaceutical composition described herein. In another aspect, the disclosure provides a method of treating an autoimmune or inflammatory disease, disorder, or condition, or a viral infection in a subject in need thereof, the method comprising administering to the subject a therapeutically effective amount of a hIL10 variant hIL10 mutein described herein or a pharmaceutical composition described herein.

In some embodiments, the present disclosure provides for the treatment or prevention of an autoimmune disease in a mammalian subject by the administration of a therapeutically effective amount of a hIL10 mutein of the present disclosure. In some embodiments, the present disclosure provides for the treatment or prevention of infectious disease, including viral and chronic viral infections, in a mammalian subject by the administration of a therapeutically effective amount of a hIL10 mutein of the present disclosure. In some embodiments, the present disclosure provides for the treatment or prevention of neoplastic disease in a mammalian subject by the administration of a therapeutically effective amount of a hIL10 mutein of the present disclosure. In some embodiments, the present disclosure provides for the treatment or prevention of neoplastic, infectious or autoimmune disease in a mammalian subject by the administration of a therapeutically effective amount of a hIL10 mutein of the present disclosure in combination with one or more supplementary therapeutic agents. In some embodiments, the hIL10 mutein administered to the mammalian subject is a monomer or dimer.

In another aspect, the disclosure provides a kit for modulating IL10-mediated signaling in a subject, or treating a health condition in a subject in need thereof. In some embodiments, the kit comprises a hIL10 variant or hIL10 mutein described herein; a nucleic acid molecule or vector comprising a nucleic acid sequence encoding an hIL10 variant or hIL10 mutein described herein; a recombinantly modified cell comprising a nucleic acid molecule or vector described herein; or a pharmaceutical composition described herein.

In some embodiments, the hIL10 muteins described herein are partial agonists of the IL10 receptor.

### BRIEF DESCRIPTION OF THE DRAWINGS

FIG. 1 provides a cartoon illustration of the principles of the SEAP assay to evaluate STAT3 induction in HEK293 cells expressing the hIL10 receptor.

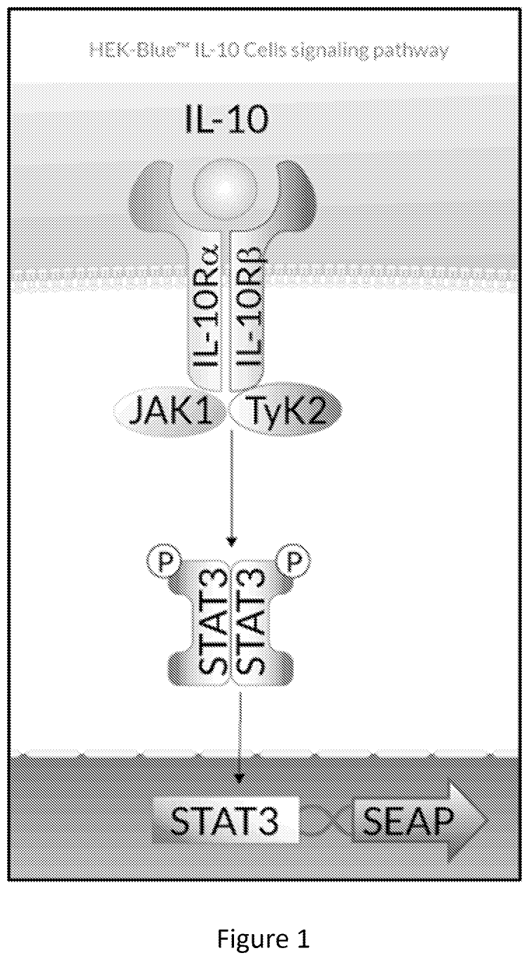

FIG. 2 provides the results of a representative STAT3 assay evaluating the activity (vertical axis) versus concentration of test article (horizontal axis) of conditioned media following recombinant expression in Expi293 cells containing an hIL10 mutein comprising a N21C substitution and an N-terminal HISx8 chelating peptide followed by a glycine-serine (“GS”) linker and wild-type hIL10 and an N-terminal HISx8 chelating peptide followed by a GS linker produced by the same method as more fully described in the Examples.

FIG. 3. provides the results of a representative STAT3 assay evaluating the activity (vertical axis) versus concentration of test article (horizontal axis) of conditioned media following recombinant expression in Expi293 cells containing an hIL10 mutein comprising an N21D substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 4 provides the results of a representative STAT3 assay evaluating the activity (vertical axis) versus concentration of test article (horizontal axis) of conditioned media in Panel A following recombinant expression in Expi293 cells containing an hIL10 mutein comprising an N21E substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 5 provides the results of a representative STAT3 assay evaluating the activity (vertical axis) versus concentration of test article (horizontal axis) of conditioned media in Panel A following recombinant expression in Expi293 cells containing an hIL10 mutein comprising a M22D substitution and an N-terminal HISx8 chelating peptide followed by a GS linker and wild-type hIL10 and an N-terminal HISx8 chelating peptide followed by a GS linker, and the same test articles following purification by metal affinity chromatography in Panel B as more fully described in the Examples.

FIG. 6 provides the results of a representative STAT3 assay evaluating the activity (vertical axis) versus concentration of test article (horizontal axis) of conditioned media in Panel A following recombinant expression in Expi293 cells containing an hIL10 mutein comprising a M22S substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 7 provides the results of a representative STAT3 assay evaluating the activity (vertical axis) versus concentration of test article (horizontal axis) of conditioned media in Panel A following recombinant expression in Expi293 cells containing an hIL10 mutein comprising a M22T substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 8 provides the results of a representative STAT3 assay evaluating the activity (vertical axis) versus concentration of test article (horizontal axis) of conditioned media in Panel A following recombinant expression in Expi293 cells containing an hIL10 mutein comprising a M22W substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 9 provides the results of a representative STAT3 assay evaluating the activity (vertical axis) versus concentration of test article (horizontal axis) of conditioned media in Panel A following recombinant expression in Expi293 cells containing an hIL10 mutein comprising a E96C substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 10 provides the results of a representative STAT3 assay evaluating the activity (vertical axis) versus concentration of test article (horizontal axis) of conditioned media following recombinant expression in Expi293 cells containing an hIL10 mutein comprising a E96F substitution and an N-terminal HISx8 chelating peptide followed by a GS linker and wild-type hIL10 and an N-terminal HISx8 chelating peptide followed by a GS linker produced by the same method as more fully described in the Examples.

FIG. 11 provides the results of a representative STAT3 assay evaluating the activity (vertical axis) versus concentration of test article (horizontal axis) of conditioned media following recombinant expression in Expi293 cells containing an hIL10 mutein comprising a E96Y substitution and an N-terminal HISx8 chelating peptide followed by a GS linker and wild-type hIL10 and an N-terminal HISx8 chelating peptide followed by a GS linker produced by the same method as more fully described in the Examples.

FIG. 12 provides the results of a representative STAT3 assay evaluating the activity (vertical axis) versus concentration of test article (horizontal axis) of conditioned media following recombinant expression in Expi293 cells containing an hIL10 mutein comprising a a E96W substitution and an N-terminal HISx8 chelating peptide followed by a GS linker and wild-type hIL10 and an N-terminal HISx8 chelating peptide followed by a GS linker produced by the same method as more fully described in the Examples.

FIG. 13 provides the results of a representative STAT3 assay evaluating the activity (vertical axis) versus concentration of test article (horizontal axis) of conditioned media following recombinant expression in Expi293 cells containing an hIL10 mutein comprising a T100C substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 14 shows conditioned media titers of hIL10 muteins comprising amino acid substitutions at position H14 as more fully described in the Examples.

FIG. 15 provides the results of a representative STAT3 assay evaluating the activity (vertical axis) versus concentration of test article (horizontal axis) of conditioned media following recombinant expression in Expi293 cells containing hIL10 muteins comprising the T100L/H14G; T100L/H14D and an N-terminal HISx8 chelating peptides followed by a GS linker and wild-type hIL10 and an N-terminal HISx8 chelating peptide followed by a GS linker produced by the same method as more fully described in the Examples.

FIG. 16 provides the results of a representative STAT3 assay evaluating the activity (vertical axis) versus concentration of test article (horizontal axis) of conditioned media following recombinant expression in Expi293 cells containing hIL10 muteins comprising the N21K/H14G; N21K/H14D and an N-terminal HISx8 chelating peptides followed by GS linkers and wild-type hIL10 and an N-terminal HISx8 chelating peptide followed by a GS linker produced by the same method as more fully described in the Examples.

FIG. 17 provides the results of a representative STAT3 assay evaluating the activity (vertical axis) versus concentration of test article (horizontal axis) of conditioned media following recombinant expression in Expi293 cells containing hIL10 muteins comprising the M22D/H14G; M22D/H14D and an N-terminal HISx8 chelating peptides followed by GS linkers and wild-type hIL10 and an N-terminal HISx8 chelating peptide followed by a GS linker produced by the same method as more fully described in the Examples.

FIG. 18 provides the results of a representative STAT3 assay evaluating the activity (vertical axis) versus concentration of test article (horizontal axis) of conditioned media following recombinant expression in Expi293 cells containing hIL10 muteins comprising the M22S/H14G; M22S/H14D and an N-terminal HISx8 chelating peptides followed by GS linkers and wild-type hIL10 and an N-terminal HISx8 chelating peptide followed by a GS linker produced by the same method as more fully described in the Examples.

FIG. 19 provides the results of a representative STAT3 assay evaluating the activity (vertical axis) versus concentration of test article (horizontal axis) of conditioned media following recombinant expression in Expi293 cells containing an hIL10 mutein comprising the M22A/H14D and an N-terminal HISx8 chelating peptide followed by a GS linker and wild-type hIL10 and an N-terminal HISx8 chelating peptide followed by a GS linker produced by the same method as more fully described in the Examples.

FIG. 20 provides the results of a representative STAT3 assay evaluating the activity (vertical axis) versus concentration of test article (horizontal axis) of conditioned media following recombinant expression in Expi293 cells containing hIL10 muteins comprising the M22W/H14G; M22W/H14D; M22W and an N-terminal HISx8 chelating peptides followed by a GS linkers and wild-type hIL10 and an N-terminal HISx8 chelating peptide followed by a a GS linker produced by the same method as more fully described in the Examples.

FIG. 21 provides the results of a representative STAT3 assay evaluating the activity (vertical axis) versus concentration of test article (horizontal axis) of conditioned media following recombinant expression in Expi293 cells containing hIL10 muteins comprising the E74K/H14G; E74K/H14D and an N-terminal HISx8 chelating peptides followed by a GS linkers and wild-type hIL10 and an N-terminal HISx8 chelating peptide produced by the same method as more fully described in the Examples.

FIG. 22 provides the results of a representative STAT3 assay evaluating the activity (vertical axis) versus concentration of test article (horizontal axis) of conditioned media following recombinant expression in Expi293 cells containing hIL10 muteins comprising the E96K/H14G; E96K/H14D and an N-terminal HISx8 chelating peptides followed by GS linkers and wild-type hIL10 and an N-terminal HISx8 chelating peptide followed by a GS linker produced by the same method as more fully described in the Examples.

FIG. 23 provides the results of a series of experiments evaluating the level of expression Expi293 cells, the concentration (nM) on the vertical axis, and with various combinations of amino acid substitutions as indicated illustrating the contribution to expression of combining amino acid substitutions with mutations at position H14 as more fully described in the Examples.

FIG. 24 provides the results of a representative STAT3 assay evaluating the activity (vertical axis) versus concentration of test article (horizontal axis) of conditioned media following recombinant expression in Expi293 cells containing an hIL10 mutein comprising an N18R substitution and an N-terminal HISx8 chelating peptide followed by a GS linker and wild-type hIL10 and a N-terminal HISx8 chelating peptide followed by a GS linker and the same test articles following purification by metal affinity chromatography in Panel B as more fully described in the Examples.

FIG. 25 provides the results of a representative STAT3 assay evaluating the activity (vertical axis) versus concentration of test article (horizontal axis) of conditioned media following recombinant expression in Expi293 cells containing an hIL10 mutein comprising an N18K substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 26 provides the results of a representative STAT3 assay evaluating the activity (vertical axis) versus concentration of test article (horizontal axis) of conditioned media following recombinant expression in Expi293 cells containing an hIL10 mutein comprising a R32N substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 27 provides the results of a representative STAT3 assay evaluating the activity (vertical axis) versus concentration of test article (horizontal axis) of conditioned media following recombinant expression in Expi293 cells containing an hIL10 mutein comprising a R32Q substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 28 provides the results of a representative STAT3 assay evaluating the activity (vertical axis) versus concentration of test article (horizontal axis) of conditioned media following recombinant expression in Expi293 cells containing an hIL10 mutein comprising a R32G substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 29 provides the results of a representative STAT3 assay evaluating the activity (vertical axis) versus concentration of test article (horizontal axis) of conditioned media following recombinant expression in Expi293 cells containing an hIL10 mutein comprising a R32C substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 30 provides the results of a representative STAT3 assay evaluating the activity (vertical axis) versus concentration of test article (horizontal axis) of conditioned media following recombinant expression in Expi293 cells containing an hIL10 mutein comprising a R32P substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 31 provides the results of a representative STAT3 assay evaluating the activity (vertical axis) versus concentration of test article (horizontal axis) of conditioned media following recombinant expression in Expi293 cells containing an hIL10 mutein comprising a R32F substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 32 provides the results of a representative STAT3 assay evaluating the activity (vertical axis) versus concentration of test article (horizontal axis) of conditioned media following recombinant expression in Expi293 cells containing an hIL10 mutein comprising a R32Y substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

FIG. 33 provides the results of a representative STAT3 assay evaluating the activity (vertical axis) versus concentration of test article (horizontal axis) of conditioned media following recombinant expression in Expi293 cells containing an hIL10 mutein comprising a S93G substitution and an N-terminal HISx8 chelating peptide followed by a GS linker and wild-type hIL10 and an N-terminal HISx8 chelating peptide followed by a GS linker and the same test articles following purification by metal affinity chromatography in Panel B as more fully described in the Examples.

### DETAILED DESCRIPTION OF THE INVENTION

To facilitate the understanding of present disclosure, certain terms and phrases are defined below as well as throughout the specification. The definitions provided herein are non-limiting and should be read in view of the knowledge of one of skill in the art.

Before the present methods and compositions are described, it is to be understood that this invention is not limited to a particular method or composition described, as such may, of course, vary. It is also to be understood that the terminology used herein is for the purpose of describing embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications, patents, published patent applications, GenBank accession numbers and UniProt reference numbers mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); AA or aa=amino acid(s); kb=kilobase(s); nt=nucleotide(s); pg=picogram; ng=nanogram; μg=microgram; mg=milligram; g=gram; kg=kilogram; dl or dL=deciliter; μl or μL=microliter; ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=once weekly; QM=once monthly; HPLC=high performance liquid chromatography; BW=body weight; U=unit; ns=not statistically significant; PBS=phosphate-buffered saline; PCR=polymerase chain reaction; HSA=human serum albumin; MSA=mouse serum albumin; DMEM=Dulbeco's Modification of Eagle's Medium; EDTA=ethylenediaminetetraacetic acid.

It will be appreciated that throughout this disclosure reference is made to amino acids according to the single letter or three letter codes.

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

### Nomenclature of Amino Acid Substitutions and Deletions

The present disclosure provides variant polypeptides comprising amino acid substitutions relative to the wild-type or parent polypeptide. The following nomenclature is used herein to refer to substitutions, deletions or insertions. Residues may be designated herein by the one-letter or three-letter amino acid code of the naturally occurring amino acid found in the wild-type molecule. In the present disclosure, the numbering of amino acid residues of human IL10 polypeptide monomers is made in reference to the number of the residue of the “mature” form of the hIL10 polypeptide monomer as provided in SEQ ID NO: 1. A deletion of an amino acid reside is referred to as “des” or the symbol “Δ” followed by the amino acid residue and its position.

Immunoglobulin, Upper Hinge and Fc Residue Numbering: There are a variety of numbering conventions that are employed with respect to the numbering of amino acid residues of immunoglobulins including Kabat numbering, Chothia numbering, EU numbering and IMGT numbering conventions. In the context of the present disclosure, the numbering of amino acid residues of immunoglobulin molecules including domains thereof including the upper hinge and Fc domain (comprising the lower hinge, CH2 and CH3 domains) is made in accordance with EU Numbering conventions. Translation of EU numbering conventions used herein to Kabat numbering, Chothia numbering, or IMGT numbering conventions is readily understood by those of skill in the art. Dondelinger, et al. (2018) Understanding the Significance and Implications of Antibody Numbering and Antigen-Binding Surface/Residue Definition Frontiers in Immunology Volume 9 Article #:2278.

### Definitions

Unless otherwise indicated, the following terms are intended to have the meaning set forth below. Other terms are defined elsewhere throughout the specification.

The term “about” refers to a value that is plus or minus 10% of a numerical value described herein, such as plus or minus 0%, 1%, 2%, 3%, 4%, 5%, 6%, 7%, 8%, 9%, or 10% of numerical value described herein. The term “about” also applies to all numerical ranges described herein. All values described herein are understood to be modified by the term “about” whether or not the term “about” is explicitly recited in reference to a given value.

Activate: As used herein the term “activate” is used in reference to a receptor or receptor complex to reflect a biological effect, directly and/or by participation in a multicomponent signaling cascade, arising from the binding of an agonist ligand to a receptor responsive to the binding of the ligand.

Activity: As used herein, the term “activity” is used with respect to a molecule to describe a property of the molecule with respect to a test system (e.g., an assay) or biological or chemical property (e.g., the degree of binding of the molecule to another molecule) or of a physical property of a material or cell (e.g., modification of cell membrane potential). Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT3 phosphorylation]/[mg protein], [proliferation]/[mg protein], plaque forming units (pfu), etc. As used herein, the term proliferative activity refers to an activity that promotes cell proliferation and replication, including dysregulated cell division such as that observed in neoplastic diseases, inflammatory diseases, fibrosis, dysplasia, cell transformation, metastasis, and angiogenesis.

Administer/Administration: The terms “administration” and “administer” are used interchangeably herein to refer the act of contacting a subject, including contacting a cell, tissue, organ, or biological fluid of the subject in vitro, in vivo or ex vivo with an agent (e.g., a hIL10 mutein or an engineered cell expressing a hIL10 mutein, a chemotherapeutic agent, an antibody, or a pharmaceutical formulation comprising one or more of the foregoing). Administration of an agent may be achieved through any of a variety of art recognized methods including but not limited to the topical administration, intravascular injection (including intravenous or intraarterial infusion), intradermal injection, subcutaneous injection, intramuscular injection, intraperitoneal injection, intracranial injection, intratumoral injection, transdermal, transmucosal, iontophoretic delivery, intralymphatic injection, intragastric infusion, intraprostatic injection, intravesical infusion (e.g., bladder), inhalation (e.g respiratory inhalers including dry-powder inhalers), intraocular injection, intraabdominal injection, intralesional injection, intraovarian injection, intracerebral infusion or injection, intracerebroventricular injection (ICVI), and the like. The term “administration” includes contact of an agent to the cell, tissue or organ as well as the contact of an agent to a fluid, where the fluid is in contact with the cell, tissue or organ.

Affinity: As used herein the term “affinity” refers to the degree of specific binding of a first molecule (e.g., a ligand) to a second molecule (e.g., a receptor) and is measured by the equilibrium dissociation constant (K_{D}), a ratio of the dissociation rate constant between the molecule and its target (K_{off}) and the association rate constant between the molecule and its target (Kₒₙ).

Agonist: As used herein, the term “agonist” refers a first agent that specifically binds a second agent (“target”) and interacts with the target to cause or promote an increase in the activation of the target. In some instances, agonists are activators of receptor proteins that modulate cell activation, enhance activation, sensitize cells to activation by a second agent, or up-regulate the expression of one or more genes, proteins, ligands, receptors, biological pathways, that may result in cell proliferation or pathways that result in cell cycle arrest or cell death such as by apoptosis. In some embodiments, an agonist is an agent that binds to a receptor and alters the receptor state resulting in a biological response that mimics the effect of the endogenous ligand of the receptor. The term “agonist” includes partial agonists, full agonists and superagonists. An agonist may be described as a “full agonist” when such agonist which leads to a substantially full biological response (i.e. the response associated with the naturally occurring ligand/receptor binding interaction) induced by receptor under study, or a partial agonist. A “superagonist” is a type of agonist that can produce a maximal response greater than the endogenous agonist for the target receptor, and thus has an activity of more than 100% of the native ligand. A super agonist is typically a synthetic molecule that exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the naturally occurring form of the molecule when evaluated at similar concentrations in a comparable assay. It should be noted that the biological effects associated with the full agonist may differ in degree and/or in kind from those biological effects of partial or superagonists. In contrast to agonists, antagonists may specifically bind to a receptor but do not result the signal cascade typically initiated by the receptor and may to modify the actions of an agonist at that receptor. Inverse agonists are agents that produce a pharmacological response that is opposite in direction to that of an agonist.

Antagonist: As used herein, the term “antagonist” or “inhibitor” refers a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. Inhibitors are molecules that decrease, block, prevent, delay activation, inactivate, desensitize, or down-regulate, e.g., a gene, protein, ligand, receptor, biological pathway including an immune checkpoint pathway, or cell.

Biological Sample: As used herein, the term “biological sample” or “sample” refers to a sample obtained (or derived) from a subject. By way of example, a biological sample comprises a material selected from the group consisting of body fluids, blood, whole blood, plasma, serum, mucus secretions, saliva, cerebrospinal fluid (CSF), bronchoalveolar lavage fluid (BALF), fluids of the eye (e.g., vitreous fluid, aqueous humor), lymph fluid, lymph node tissue, spleen tissue, bone marrow, tumor tissue, including immunoglobulin enriched or cell-type specific enriched fractions derived from one or more of such tissues.

Comparable: As used herein, the term “comparable” is used to describe the degree of difference in two measurements of an evaluable quantitative or qualitative parameter. For example, where a first measurement of an evaluable quantitative parameter and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 35%, alternatively by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard.

Conservative Amino Acid Substitution: As used herein, the term “conservative amino acid substitution” refers to an amino acid replacement that changes a given amino acid to a different amino acid with similar biochemical properties (e.g., charge, hydrophobicity, and size). For example, the amino acids in each of the following groups can be considered as conservative amino acids of each other: (1) hydrophobic amino acids: alanine, isoleucine, leucine, tryptophan, phenylalanine, valine, proline, and glycine; (2) polar amino acids: glutamine, asparagine, histidine, serine, threonine, tyrosine, methionine, and cysteine; (3) basic amino acids: lysine and arginine; and (4) acidic amino acids: aspartic acid and glutamic acid.

Corresponding To: As used herein, the terms “correspondence” or “corresponding to” in the context of an amino acid or nucleic acid sequence refers to the equivalent position of a reference sequence that is aligned with one or more other sequences to maximize the percentage of sequence identity. For example, an “amino acid position corresponding to amino acid position [X]” of a specified IL10 polypeptide refers to equivalent positions, based on alignment, in other IL10 polypeptides, including structural homologues and variants. The corresponding position can be based on a reference, wild-type or parental sequence, for example the amino acid sequence of SEQ ID NO:1.

Derived From: As used herein, the term “derived from” in the context of an amino acid sequence is meant to indicate that the polypeptide or nucleic acid has a sequence that is based on that of a reference polypeptide or nucleic acid and is not meant to be limiting as to the source or method in which the protein or nucleic acid is made. By way of example, the term “derived from” includes homologs or variants of reference amino acid or DNA sequences.

Effective Concentration (EC): As used herein, the terms “effective concentration” or its abbreviation “EC” are used interchangeably to refer to the concentration of an agent in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation EC is provided with a subscript (e.g., EC₄₀, EC₅₀, etc.) the subscript refers to the percentage of the Emax of the biological response observed at that concentration. For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results in the maximal (100%) response of a measurable parameter in response to such agent. Similarly, the term EC₅₀ (which is commonly used in the field of pharmacodynamics) refers to the concentration of an agent sufficient to result in the half-maximal (about 50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect.

Enriched: As used herein in the term “enriched” refers to a sample that is non-naturally manipulated so that a species (e.g., a molecule or cell) of interest is present in: (a) a greater concentration (e.g., at least 3-fold greater, alternatively at least 5-fold greater, alternatively at least 10-fold greater, alternatively at least 50-fold greater, alternatively at least 100-fold greater, or alternatively at least 1000-fold greater) than the concentration of the species in the starting sample, such as a biological sample (e.g., a sample in which the molecule naturally occurs or in which it is present after administration); or (b) a concentration greater than the environment in which the molecule was made (e.g., a recombinantly modified bacterial or mammalian cell).

Extracellular Domain: As used herein the term “extracellular domain” or its abbreviation “ECD” refers to the portion of a cell surface protein (e.g., a cell surface receptor) which is external to of the plasma membrane of a cell. The cell surface protein may be transmembrane protein, a cell surface or membrane associated protein.

Identity: The term “identity,” as used herein in reference to polypeptide or DNA sequences, refers to the subunit sequence identity between two molecules. When a subunit position in both of the molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as BLAST 2.0 algorithms, which are described in Altschul et al. (1990) J. Mol. Biol. 215: 403-410 and Altschul, et al. (1977) Nucleic Acids Res. 25: 3389-3402. Software for performing BLAST analyses is publicly available through the National Center for Biotechnology Information (NCBI) web site. The algorithm involves first identifying high scoring sequence pairs (HSPs) by identifying short words of length W of the query sequence, which either match or satisfy some positive-valued threshold score “T” when aligned with a word of the same length in a database sequence. T is referred to as the neighborhood word score threshold (Altschul, et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (the reward score for a pair of matching residues; always >0) and “N” (the penalty score for mismatching residues; always <0). For amino acid sequences, a scoring matrix is used to calculate the cumulative score. Extension of the word hits in each direction are halted when: (a) the cumulative alignment score falls off by the quantity X from its maximum achieved value; the cumulative score goes to zero or below, due to the accumulation of one or more negative-scoring residue alignments; or (b) the end of either sequence is reached. The BLAST algorithm parameters “W”, “T”, and “X” determine the sensitivity and speed of the alignment. The BLASTN program (for nucleotide sequences) functions similarly but uses as defaults a word size (“W”) of 28, an expectation (“E”) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, (1989) PNAS(USA) 89:10915-10919).

In An Amount Sufficient Amount to Effect a Response: As used herein the phrase “in an amount sufficient to cause a response” is used in reference to the amount of a test agent sufficient to provide a detectable change in the level of an indicator measured before (e.g., a baseline level) and after the application of a test agent to a test system. In some embodiments, the test system is a cell, tissue or organism. In some embodiments, the test system is an in vitro test system such as a fluorescent assay. In some embodiments, the test system is an in vivo system which involves the measurement of a change in the level a parameter of a cell, tissue, or organism reflective of a biological function before and after the application of the test agent to the cell, tissue, or organism. In some embodiments, the indicator is reflective of biological function or state of development of a cell evaluated in an assay in response to the administration of a quantity of the test agent. In some embodiments, the test system involves the measurement of a change in the level an indicator of a cell, tissue, or organism reflective of a biological condition before and after the application of one or more test agents to the cell, tissue, or organism. The term “in an amount sufficient to effect a response” may be sufficient to be a therapeutically effective amount but may also be more or less than a therapeutically effective amount.

In Need of Treatment: The term “in need of treatment” as used herein refers to a judgment made by a physician or other caregiver with respect to a subject that the subject requires or will potentially benefit from treatment. This judgment is made based on a variety of factors that are in the realm of the physician's or caregiver's expertise.

In Need of Prevention: As used herein the term “in need of prevention” refers to a judgment made by a physician or other caregiver with respect to a subject that the subject requires or will potentially benefit from preventative care. This judgment is made based upon a variety of factors that are in the realm of a physician's or caregiver's expertise.

Inhibitor: As used herein the term “inhibitor” refers to a molecule that decreases, blocks, prevents, delays activation of, inactivates, desensitizes, or down-regulates, e.g., a gene, protein, ligand, receptor, or cell. An inhibitor can also be defined as a molecule that reduces, blocks, or inactivates a constitutive activity of a cell or organism.

Intracellular Domain: As used herein the term “intracellular domain” or its abbreviation “ICD” refers to the portion of a cell surface protein (e.g., a cell surface receptor) which is inside of the plasma membrane of a cell. The ICD may include the entire cytoplasmic portion of a transmembrane protein or membrane associated protein, or intracellular protein.

Isolated: As used herein the term “isolated” is used in reference to a polypeptide of interest that, if naturally occurring, is in an environment different from that in which it can naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was synthesized, for example isolated from a recombinant cell culture comprising cells engineered to express the polypeptide or by a solution resulting from solid phase synthetic means.

Ligand: As used herein, the term “ligand” refers to a molecule that specifically binds a receptor and causes a change in the receptor so as to effect a change in the activity of the receptor or a response in cell that expresses that receptor. In one embodiment, the term “ligand” refers to a molecule or complex thereof that can act as an agonist or antagonist of a receptor. As used herein, the term “ligand” encompasses natural and synthetic ligands. “Ligand” also encompasses small molecules, peptide mimetics of cytokines and antibodies. The complex of a ligand and receptor is termed a “ligand-receptor complex.” A ligand may comprise one domain of a polyprotein or fusion protein (e.g., either domain of an antibody/ligand fusion protein).

Modified: As used herein, the term “modified” refers to a molecule, such as a polypeptide, whose structure has been changed relative to an unmodified parental molecule. A modified polypeptide typically retains one or more activities or functions of the unmodified parental molecule. For example, a hIL10 mutein monomer can activate IL10 signaling in a cell expressing the IL10 receptor as part of a homodimer, but can have improved properties relative to the unmodified polypeptide. The term modified includes amino acid substitutions that are not present in a parental or wild-type IL10, and includes variants and muteins of an IL10 polypeptide.

Modulate: As used herein, the terms “modulate”, “modulation” and the like refer to the ability of a test agent to cause a response, either positive or negative or directly or indirectly, in a system, including a biological system, or biochemical pathway. The term modulator includes both agonists (including partial agonists, full agonists and superagonists) and antagonists.

Mutein: As used herein, the term “mutein” refers to a protein or polypeptide having an altered or modified amino acid sequence. The term also includes a nucleic acid that encodes a protein or polypeptide having an altered or modified amino acid sequence.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

The term “one or more amino acid substitutions” refers to a single amino acid substitution, or one, two, three, four, five or more amino acid substitutions in a hIL10 monomer of the disclosure.

Operably Linked: The term “operably linked” is used herein to refer to the relationship between molecules, typically polypeptides or nucleic acids, which are arranged in a construct such that each of the functions of the component molecules is retained although the operable linkage may result in the modulation of the activity, either positively or negatively, of the individual components of the construct. For example, the operable linkage of a polyethylene glycol (PEG) molecule to a wild-type protein may result in a construct where the biological activity of the protein is diminished relative to the to the wild-type molecule, however the two are nevertheless considered operably linked. When the term “operably linked” is applied to the relationship of multiple nucleic acid sequences encoding differing functions, the multiple nucleic acid sequences when combined into a single nucleic acid molecule that, for example, when introduced into a cell using recombinant technology, provides a nucleic acid which is capable of effecting the transcription and/or translation of a particular nucleic acid sequence in a cell. For example, the nucleic acid sequence encoding a signal sequence may be considered operably linked to DNA encoding a polypeptide if it results in the expression of a preprotein whereby the signal sequence facilitates the secretion of the polypeptide; a promoter or enhancer is considered operably linked to a coding sequence if it affects the transcription of the sequence; or a ribosome binding site is considered operably linked to a coding sequence if it is positioned so as to facilitate translation. Generally, in the context of nucleic acid molecules, the term “operably linked” means that the nucleic acid sequences being linked are contiguous, and, in the case of a secretory leader or associated subdomains of a molecule, contiguous and in reading phase. However, certain genetic elements such as enhancers may function at a distance and need not be contiguous with respect to the sequence to which they provide their effect but nevertheless may be considered operably linked.

Parent Polypeptide: As used herein, the terms “parent polypeptide” or “parent protein” are used interchangeably to designate the source of a second polypeptide (e.g., a derivative, mutein or variant) which is modified with respect to a first “parent” polypeptide. In some instances, the parent polypeptide is a wild-type or naturally occurring form of a protein. In some instance, the parent polypeptide may be a modified form a naturally occurring protein that is further modified. The term “parent polypeptide” may refer to the polypeptide itself or compositions that comprise the parent polypeptide (e.g., glycosylated or PEGylated forms and/or fusion proteins comprising the parent polypeptide). The term parent polypeptide can also be used interchangeably with “reference polypeptide.”

Partial Agonist: As used herein, the term “partial agonist” refers to a molecule that specifically binds that bind to and activate a given receptor but possess only partial activation the receptor relative to a full agonist. Partial agonists may display both agonistic and antagonistic effects. For example, when both a full agonist and partial agonist are present, the partial agonist acts as a competitive antagonist by competing with the full agonist for the receptor binding resulting in net decrease in receptor activation relative to the contact of the receptor with the full agonist in the absence of the partial agonist. Partial agonists can be used to activate receptors to give a desired submaximal response in a subject when inadequate amounts of the endogenous ligand are present, or they can reduce the overstimulation of receptors when excess amounts of the endogenous ligand are present. The maximum response (Eₘₐₓ) produced by a partial agonist is called its intrinsic activity and may be expressed on a percentage scale where a full agonist produced a 100% response. An partial agonist may have greater than 10% but less than 100%, alternatively greater than 20% but less than 100%, alternatively greater than 30% but less than 100%, alternatively greater than 40% but less than 100%, alternatively greater than 50% but less than 100%, alternatively greater than 60% but less than 100%, alternatively greater than 70% but less than 100%, alternatively greater than 80% but less than 100%, or alternatively greater than 90% but less than 100%, of the activity of the reference polypeptide when evaluated at similar concentrations in a given assay system.

Polypeptide: As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The term polypeptide include fusion proteins, including, but not limited to, fusion proteins with a heterologous amino acid sequence; fusion proteins with heterologous and homologous leader sequences; fusion proteins with or without N-terminal methionine residues; fusion proteins with amino acid sequences that facilitate purification such as chelating peptides; fusion proteins with immunologically tagged proteins; fusion proteins comprising a peptide with immunologically active polypeptide fragment (e.g., antigenic diphtheria or tetanus toxin or toxoid fragments) and the like.

Prevent: As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof. A course of action to prevent a disease, disorder or condition in a subject is typically applied in the context of a subject who is predisposed to developing a disease, disorder or condition due to genetic, experiential or environmental factors of developing a particular disease, disorder or condition. In certain instances, the terms “prevent”, “preventing”, “prevention” are also used to refer to the slowing of the progression of a disease, disorder or condition from an existing state to a more deleterious state.

Receptor: As used herein, the term “receptor” refers to a polypeptide having a domain that specifically binds a ligand that binding of the ligand results in a change to at least one biological property of the polypeptide. In some embodiments, the receptor is a cell membrane associated protein that comprises and extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain (TM). The binding of a cognate ligand to the receptor results in a conformational change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface receptor that is not associated with any intracellular signaling alone but upon ligand binding facilitates the formation of a heteromultimeric (including heterodimeric, heterotrimeric, etc.) or homomultimeric (including homodimeric, homotrimeric, homotetrameric, etc.) complex that results in a measurable biological effect in the cell such as activation of an intracellular signaling cascade (e.g., the Jak/STAT pathway). In some embodiments, a receptor is a membrane spanning single chain polypeptide comprising ECD, TM and ICD domains wherein the ECD, TM and ICD domains are derived from the same or differing naturally occurring receptor variants or synthetic functional equivalents thereof.

Recombinant: As used herein, the term “recombinant” is used as an adjective to refer to the method by which a polypeptide, nucleic acid, or cell was modified using recombinant DNA technology. A “recombinant protein” is a protein produced using recombinant DNA technology and is frequently abbreviated with a lower case “r” preceding the protein name to denote the method by which the protein was produced (e.g., recombinantly produced human growth hormone is commonly abbreviated “rhGH”). Similarly a cell is referred to as a “recombinant cell” if the cell has been modified by the incorporation (e.g., transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation) where the change is correlated with the activation, stimulation, or treatment, with or contact with exogenous agents or internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects. A “response” may be evaluated in vitro such as through the use of assay systems, surface plasmon resonance, enzymatic activity, mass spectroscopy, amino acid or protein sequencing technologies. A “response” may be evaluated in vivo quantitatively by evaluation of objective physiological parameters such as body temperature, bodyweight, tumor volume, blood pressure, results of X-ray or other imaging technology or qualitatively through changes in reported subjective feelings of well-being, depression, agitation, or pain. In some embodiments, the level of proliferation of CD3 activated primary human T-cells may be evaluated in a bioluminescent assay that generates a luminescent signal that is proportional to the amount of ATP present which is directly proportional to the number of viable cells present in culture as described in Crouch, et al. (1993) J. Immunol. Methods 160: 81-8 or using commercially available assays such as the CellTiter-Glo® 2.0 Cell Viability Assay or CellTiter-Glo® 3D Cell Viability kits commercially available from Promega Corporation, Madison WI 53711 as catalog numbers G9241 and G9681 in substantial accordance with the instructions provided by the manufacturer. In some embodiments, the level of activation of T cells in response to the administration of a test agent may be determined by flow cytometric methods as described as determined by the level of STAT (e.g., STAT1, STAT3, STAT5) phosphorylation in accordance with methods well known in the art.

Significantly Reduced Binding: As used herein, the term “exhibits significantly reduced binding” is used with respect a variant of a first molecule (e.g., a ligand or antibody) which exhibits a significant reduction in the affinity for a second molecule (e.g., receptor or antigen) relative the parent form of the first molecule. With respect to antibody variants, an antibody variant “exhibits significantly reduced binding” if the affinity of the variant antibody for an antigen if the variant binds to the native form of the receptor with and affinity of less than 20%, alternatively less than about 10%, alternatively less than about 8%, alternatively less than about 6%, alternatively less than about 4%, alternatively less than about 2%, alternatively less than about T %, or alternatively less than about 0.5% of the parent antibody from which the variant was derived. Similarly, with respect to variant ligands, a variant ligand “exhibits significantly reduced binding” if the affinity of the variant ligand binds to a receptor with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent ligand from which the variant ligand was derived. Similarly, with respect to variant receptors, a variant ligand “exhibits significantly reduced binding” if the affinity of the variant receptors binds to a with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent receptor from which the variant receptor was derived.

Specifically Binds: As used herein the term “specifically binds” refers to the degree of affinity for which a first molecule exhibits with respect to a second molecule. In the context of binding pairs (e.g., ligand/receptor, antibody/antigen) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample. In a particular embodiment, where the first molecule of the binding pair is an antibody, the antibody specifically binds to the antigen (or antigenic determinant (epitope) of a protein, antigen, ligand, or receptor) if the equilibrium dissociation constant (K_{D}) between antibody and the antigen is lesser than about 10⁻⁶ M, alternatively lesser than about 10⁻⁸ M, alternatively lesser than about 10⁻¹⁰ M, alternatively lesser than about 10⁻¹¹ M, lesser than about 10⁻¹² M as determined by, e.g., Scatchard analysis (Munsen, et al. (1980) Analyt. Biochem. 107:220-239). In one embodiment where the ligand is an IL10 molecule or variant thereof and the receptor comprises an IL10Rb, the IL10 molecule or variant thereof specifically binds if the equilibrium dissociation constant of the IL10 molecule/IL10Rb ECD is greater than about 10⁻⁵ M, alternatively greater than about 10⁻⁶ M, alternatively greater than about 10⁷ M, alternatively greater than about 10⁻⁸ M, alternatively greater than about 10⁻⁹ M, alternatively greater than about 10⁻¹⁰ M, or alternatively greater than about 10⁻¹¹ M. Specific binding may be assessed using techniques known in the art including but not limited to competition ELISA assays, radioactive ligand binding assays (e.g., saturation binding, Scatchard plot, nonlinear curve fitting programs and competition binding assays); non-radioactive ligand binding assays (e.g., fluorescence polarization (FP), fluorescence resonance energy transfer (FRET); liquid phase ligand binding assays (e.g., real-time polymerase chain reaction (RT-qPCR), and immunoprecipitation); and solid phase ligand binding assays (e.g., multiwell plate assays, on-bead ligand binding assays, on-column ligand binding assays, and filter assays)) and surface plasmon resonance assays (see, e.g., Drescher et al., (2009) Methods Mol Biol 493:323-343 with commercially available instrumentation such as the Biacore 8K, Biacore 8K+, Biacore S200, Biacore T200 (Cytiva, 100 Results Way, Marlborough MA 01752).

Subject: The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Substantially Pure: As used herein, the term “substantially pure” indicates that a component of a composition makes up greater than about 50%, alternatively greater than about 60%, alternatively greater than about 70%, alternatively greater than about 80%, alternatively greater than about 90%, alternatively greater than about 95% of the total content of the composition. A protein that is “substantially pure” comprises greater than about 50%, alternatively greater than about 60%, alternatively greater than about 70%, alternatively greater than about 80%, alternatively greater than about 90%, alternatively greater than about 95% of the total content of the composition.

Suffering From: As used herein, the term “suffering from” refers to a determination made by a physician with respect to a subject based on the available objective or subjective information accepted in the field for the identification of a disease, disorder or condition including but not limited to X-ray, CT-scans, conventional laboratory diagnostic tests (e.g., blood count, etc.), genomic data, protein expression data, immunohistochemistry, that the subject requires or will benefit from treatment. The term suffering from is typically used in conjunction with a particular disease state such as “suffering from a neoplastic disease” refers to a subject which has been diagnosed with the presence of a neoplasm.

T-cell: As used herein the term “T-cell” or “T cell” is used in its conventional sense to refer to a lymphocytes that differentiates in the thymus, possess specific cell-surface antigen receptors, and include some that control the initiation or suppression of cell-mediated and humoral immunity and others that lyse antigen-bearing cells. In some embodiments the T cell includes without limitation naïve CD8⁺ T cells, cytotoxic CD8⁺ T cells, naïve CD4⁺ T cells, helper T cells, e.g., T_{H}1, T_{H}2, T_{H}9, T_{H}11, T_{H}22, T_{FH}; regulatory T cells, e.g., T_{R}1, Tregs, inducible Tregs; memory T cells, e.g., central memory T cells, effector memory T cells, NKT cells, tumor infiltrating lymphocytes (TILs) and engineered variants of such T-cells including but not limited to CAR-T cells, recombinantly modified TILs and TCR-engineered cells. In some embodiments the T cell is a T cell expressing the IL10Rb isoform referred to interchangeably as IL10Rb cell, IL10Rb+ cell, IL10Rb T cell, or IL10Rb+ T cell.

Terminus/Terminal: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” refers to the position of a first amino acid residue relative to a second amino acid residue in a contiguous polypeptide sequence, the first amino acid being closer to the N-terminus of the polypeptide. “Immediately C-terminal” refers to the position of a first amino acid residue relative to a second amino acid residue in a contiguous polypeptide sequence, the first amino acid being closer to the C-terminus of the polypeptide.

Therapeutically Effective Amount: As used herein to the phrase “therapeutically effective amount” refers to the quantity of an agent when administered to a subject, either alone or as part of a pharmaceutical composition or treatment regimen, in a single dose or as part of a series of doses, provides a positive effect on any quantitative or qualitative symptom, aspect, or characteristic of a disease, disorder or condition. A therapeutically effective amount can be ascertained by measuring relevant physiological effects, and it may be adjusted in connection with a dosing regimen and in response to diagnostic analysis of the subject's condition. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-γ, granzyme, and the like), reduction in serum tumor markers, improvement in Response Evaluation Criteria In Solid Tumors (RECIST), improvement in Immune-Related Response Criteria (irRC), increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that that are relied upon by clinicians in the field for the assessment of an improvement in the condition of the subject in response to administration of an agent. In one embodiment, a therapeutically effective amount is an amount of an agent when used alone or in combination with another agent provides an provides a positive effect on any quantitative or qualitative symptom, aspect, or characteristic of a disease, disorder or condition and does not result in non-reversible serious adverse events in the course of administration of the agent to the mammalian subject.

Treat: The terms “treat”, “treating”, treatment” and the like refer to a course of action (such as contacting the subject with pharmaceutical composition comprising a hIL10 variant polypeptide monomer alone or in combination with a supplementary agent) that is initiated with respect to a subject in response to a diagnosis that the subject is suffering from a disease, disorder or condition, or a symptom thereof, the course of action being initiated so as to eliminate, reduce, suppress, mitigate, or ameliorate, either temporarily or permanently, at least one of: (a) the underlying causes of such disease, disorder, or condition afflicting a subject; and/or (b) at least one of the symptoms associated with such disease, disorder, or condition. In some embodiments, treating includes a course of action taken with respect to a subject suffering from a disease where the course of action results in the inhibition (e.g., arrests the development of the disease, disorder or condition or ameliorates one or more symptoms associated therewith) of the disease in the subject.

Variant: The terms “variant”, “protein variant” or “variant protein” or “variant polypeptide” are used interchangeably herein to refer to a polypeptide that differs from a parent polypeptide by virtue of at least one amino acid modification, substitution, or deletion. The parent polypeptide may be a naturally occurring or wild-type (WT) polypeptide or may be a modified version of a WT polypeptide. The term variant polypeptide may refer to the polypeptide itself, a composition comprising the polypeptide, or the nucleic acid sequence that encodes it. In some embodiments, the variant polypeptide comprises from about one to about ten, alternatively about one to about eight, alternatively about one to about seven, alternatively about one to about five, alternatively about one to about four, alternatively from about one to about three alternatively from one to two amino acid modifications, substitutions, or deletions, or alternatively a single amino acid amino acid modification, substitution, or deletion compared to the parent polypeptide. A variant may be at least about 99% identical, alternatively at least about 98% identical, alternatively at least about 97% identical, alternatively at least about 95% identical, or alternatively at least about 90% identical to the parent polypeptide from which the variant is derived.

Wild Type: By “wild type” or “WT” or “native” herein is meant an amino acid sequence or a nucleotide sequence that is found in nature, including allelic variations. A wild-type protein, polypeptide, antibody, immunoglobulin, IgG, etc. has an amino acid sequence or a nucleotide sequence that has not been modified by the hand of man.

As used herein, the term “percent (%) sequence identity” used in the context of nucleic acids or polypeptides, refers to a sequence that has at least 50% sequence identity with a reference sequence. Alternatively, percent sequence identity can be any integer from 50% to 100%. In some embodiments, a sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined with BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned.

Algorithms that are suitable for determining percent sequence identity and sequence similarity are the BLAST and BLAST 2.0 algorithms, which are described in Altschul et al. (1990) J Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

It will be understood that individual embodiments, which are separately described herein for clarity and brevity, can be combined without limitation. Thus, the present disclosure includes one or more, or all, combinations of the embodiments described herein as if each and every combination was individually and explicitly disclosed. This also applies to any and all sub-combinations of the embodiments disclosed herein, such that the present disclosure includes one or more, or all, sub-combinations of the embodiments described herein as if each and every sub-combination was individually and explicitly disclosed.

### Human IL10:

Human IL10 (hIL10) is non-covalently linked homodimeric protein comprising two identical subunits. Each hIL10 monomer is expressed as a 178 amino acid pre-protein comprising an 18 amino acid signal sequence (SEQ ID NO:2) which is post-translationally removed to render a 160 amino acid mature protein. The canonical amino acid sequence of the mature (“wild-type”) IL10 protein (UniProt Reference No. P22301) without the signal sequence (corresponding to amino acids 19-178 of the pre-protein) has the amino acid sequence:

(SEQ ID NO: 1)
SPGQGTQSENSCTHFPGNLPNMLRDLRDAFSRVKTFFQMKDQLDNLL
LKESLLEDFKGYLGCQALSEMIQFYLEEVMPQAENQDPDIKAHVNSLG
ENLKTLRLRLRRCHRFLPCENKSKAVEQVKNAFNKLQEKGIYKAMSE
FDIFINYIEAYMTMKIRN

hIL10 Monomer Variants

In some embodiments, the present disclosure provides hIL10 monomer variants comprising one or more amino acid substitutions at positions H14, N18, N21, M22, D25, R32, S93, E96, and T100 numbered in accordance with SEQ ID NO:1.

In some embodiments the hIL10 monomer variant has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:1 and comprises one or more amino acid substitutions at positions corresponding to residues H14, N18, N21, M22, D25, R32, S93, E96, and T100 numbered in accordance with SEQ ID NO:1. In some embodiments, the hIL10 monomer variant comprises one or more amino acid substitutions at a position corresponding to residues H14, N18, N21, M22, D25, R32, S93, E96, and T100 of SEQ ID NO:1, wherein the amino acid sequence of the hIL10 monomer variant has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:1.

In some embodiments, the present disclosure provides an hIL10 monomer variant having at least 70% sequence identity (e.g., at least 70%, 75%, 80%, 85%, 90%, 91%, 92%, 93%, 94%, 95%, 96%, 97%, 98%, or 99% sequence identity) to SEQ ID NO:1, the hIL10 monomer variant comprising one or more amino substitutions at positions corresponding to residues selected from the group consisting of H14, N18, N21, M22, D25, R32, S93, E96, and T100 of SEQ ID NO:1, wherein:
the amino acid substitution at position H14 is selected from the group consisting of H14C, H14F, H14P, H14W and H14G;
the amino acid substitution at position N18 is selected from the group consisting of N18R and N18K;
the amino acid substitution at position N21 is selected from the group consisting of N21C, N21D and N21E
the amino acid substitution at position M22 is selected from the group consisting of M22D, M22S, M22T, and M22W;
the amino acid substitution at position D25 is selected from the group consisting of D25P and D25Q;
the amino acid substitution at position R32 is selected from the group consisting of R32N, R32Q, R32G, R32C, R32P, R32F and R32Y;
the amino acid substitution at position S93 is S93G;
the amino acid substitution at position E96 is selected from the group consisting of E96C, E96F, E96Y, and E96W; and
the amino acid substitution at position T100 is T100C.

In some embodiments, the hIL10 monomer variant comprises one or more amino acid substitutions at a position corresponding to residues H14, N18, N21, M22, D25, R32, S93, E96, and T100 of SEQ ID NO:1, wherein the amino acid sequence of the hIL10 monomer variant has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:1, wherein:
the amino acid substitution at position H14 is selected from the group consisting of H14C, H14F, H14P, H14W and H14G;
the amino acid substitution at position N18 is selected from the group consisting of N18R and N18K;
the amino acid substitution at position N21 is selected from the group consisting of N21C, N21D and N21E
the amino acid substitution at position M22 is selected from the group consisting of M22D, M22S, M22T, and M22W;
the amino acid substitution at position D25 is selected from the group consisting of D25P and D25Q;
the amino acid substitution at position R32 is selected from the group consisting of R32N, R32Q, R32G, R32C, R32P, R32F and R32Y;
the amino acid substitution at position S93 is S93G;
the amino acid substitution at position E96 is selected from the group consisting of E96C, E96F, E96Y, and E96W; and
the amino acid substitution at position T100 is T100C.

In some embodiments, the present disclosure provides an hIL10 monomer variant having at least 70% sequence identity (e.g., at least 70%, 75%, 80%, 85%, 90%, 91%, 92%, 93%, 94%, 95%, 96%, 97%, 98%, or 99% sequence identity) to SEQ ID NO:1, the hIL10 monomer variant comprising one or more amino substitutions at positions corresponding to residues selected from the group consisting of H14, N18, N21, M22, D25, R32, S93, E96, and T100 of SEQ ID NO:1, wherein:
the amino acid substitution at position H14 is selected from the group consisting of H14C, H14F, H14P, H14W and H14G;
the amino acid substitution at position N18 is selected from the group consisting of N18R and N18K;
the amino acid substitution at position N21 is selected from the group consisting of N21C, N21D and N21E
the amino acid substitution at position M22 is selected from the group consisting of M22D, M22S, M22T, and M22W;
the amino acid substitution at position D25 is selected from the group consisting of D25P and D25Q;
the amino acid substitution at position R32 is selected from the group consisting of R32N, R32Q, R32G, R32C, R32P, R32F and R32Y;
the amino acid substitution at position S93 is S93G;
the amino acid substitution at position E96 is selected from the group consisting of E96C, E96F, E96Y, and E96W;
the amino acid substitution at position T100 is T100C,
   and further comprising one or more amino substitutions selected from the group consisting of:
H14A, H14D, H14E, H14I, H14K, H14L, H14M, H14N, H14Q, H14R, H14S, H14T, H14Y, and H14V;
N18Y, N18F, N18A, N18D, N18E, N18L, N18V, N18S, N18T, N18I, N18V, N18M, and N18H;
N21A, N21R, N21Q, N21H, N21K, N21S, N21V, N21I, N21L, N21M, and N21T,
M22A, M22V, M22I, M22L, M22N, and M22Q;
R24E, R24D, R24N, R24Q, R24A, R24S, and R24T;
D25A, D25N, D25H, D25I, D25K, D25L, and D25V;
D28A, D28E, D28L, D28V, D28S, D28T, D28I, D28V, D28M, D28H, D28K, and D28R;
R32A, R32D, R32E, R32L, R32V, R32S, R32T, R32I, R32V, R32M, and R32H;
E74A, E74D, E74L, E74V, E74S, E74T, E74I, E74V, E74M, E74H, E74K, and E74R;
H90A, H90D, H90E, H90I, H90K, H90L, H90M, H90N, H90Q, H90R, H90S, H90T, H90Y, and H90V;
N92D, N92Q, N92E, N92H, N92K, N92S, N92V, N92I, N92L, N92M, N92T, and N92A;
S93E, S93A, S93R, S93N, S93D, S93Q, S93E, S93I, S93L, S93K, S93M, and S93V;
E96A, E96N, E96D, E96Q, E96H, E96K, and E96S;
T100D, T100V, T100E, T100A, T100R, T100N, T100Q, T100E, T100I, T100L, T100K, T100M, and T100S; and
R104A, R104W, R104Y, R104F, R104H, R104D, R104E, R104N, R104Q, R104S, R104T, R104I, R104L, R104V, and R104M, numbered according to SEQ ID NO:1.

In some embodiments, the hIL10 monomer variant comprises one or more amino acid substitutions at a position corresponding to residues H14, N18, N21, M22, D25, R32, S93, E96, and T100 of SEQ ID NO:1, wherein the amino acid sequence of the hIL10 monomer variant has at least 70% sequence identity (e.g., at least 70%, 75%, 80%, 85%, 90%, 91%, 92%, 93%, 94%, 95%, 96%, 97%, 98%, or 99% sequence identity) to SEQ ID NO:1, wherein::
the amino acid substitution at position H14 is selected from the group consisting of H14C, H14F, H14P, H14W and H14G;
the amino acid substitution at position N18 is selected from the group consisting of N18R and N18K;
the amino acid substitution at position N21 is selected from the group consisting of N21C, N21D and N21E
the amino acid substitution at position M22 is selected from the group consisting of M22D, M22S, M22T, and M22W;
the amino acid substitution at position D25 is selected from the group consisting of D25P and D25Q;
the amino acid substitution at position R32 is selected from the group consisting of R32N, R32Q, R32G, R32C, R32P, R32F and R32Y;
the amino acid substitution at position S93 is S93G;
the amino acid substitution at position E96 is selected from the group consisting of E96C, E96F, E96Y, and E96W;
the amino acid substitution at position T100 is T100C,
   and further comprising one or more amino substitutions selected from the group consisting of:
H14A, H14D, H14E, H14I, H14K, H14L, H14M, H14N, H14Q, H14R, H14S, H14T, H14Y, and H14V;
N18Y, N18F, N18A, N18D, N18E, N18L, N18V, N18S, N18T, N18I, N18V, N18M, and N18H;
N21A, N21R, N21Q, N21H, N21K, N21S, N21V, N21I, N21L, N21M, and N21T,
M22A, M22V, M22I, M22L, M22N, and M22Q;
R24E, R24D, R24N, R24Q, R24A, R24S, and R24T;
D25A, D25N, D25H, D25I, D25K, D25L, and D25V;
28A, D28E, D28L, D28V, D28S, D28T, D28I, D28V, D28M, D28H, D28K, and D28R;
R32A, R32D, R32E, R32L, R32V, R32S, R32T, R32I, R32V, R32M, and R32H;
E74A, E74D, E74L, E74V, E74S, E74T, E74I, E74V, E74M, E74H, E74K, and E74R;
H90A, H90D, H90E, H90I, H90K, H90L, H90M, H90N, H90Q, H90R, H90S, H90T, H90Y, and H90V;
N92D, N92Q, N92E, N92H, N92K, N92S, N92V, N92I, N92L, N92M, N92T, and N92A;
S93E, S93A, S93R, S93N, S93D, S93Q, S93E, S93I, S93L, S93K, S93M, and S93V;
E96A, E96N, E96D, E96Q, E96H, E96K, and E96S;
T100D, T100V, T100E, T100A, T100R, T100N, T100Q, T100E, T100I, T100L, T100K, T100M, and T100S; and
R104A, R104W, R104Y, R104F, R104H, R104D, R104E, R104N, R104Q, R104S, R104T, R104I, R104L, R104V, and R104M, numbered according to SEQ ID NO:1.

In some embodiments, hIL10 monomer variant comprises an amino acid sequence having at least 70% sequence identity (e.g., at least 70%, 75%, 80%, 85%, 90%, 91%, 92%, 93%, 94%, 95%, 96%, 97%, 98%, or 99% sequence identity) to a sequence comprising a deletion of one to 10 contiguous amino acids (e.g., a deletion of 1, 2, 3, 4, 5, 6, 7, 8, 9 or 10 contiguous amino acids) from the amino terminus of SEQ ID NO:1. In some embodiments, the hIL10 monomer variant comprises an amino acid sequence having at least 70% sequence identity (e.g., at least 70%, 75%, 80%, 85%, 90%, 91%, 92%, 93%, 94%, 95%, 96%, 97%, 98%, or 99% sequence identity) to a sequence comprising a deletion of one to 10 contiguous amino acids (e.g., a deletion of 1, 2, 3, 4, 5, 6, 7, 8, 9 or 10 contiguous amino acids) from the amino acid sequence SPGQGTQSEN located at the amino terminus of SEQ ID NO:1 (e.g., a deletion of S, SP, SPG, SPGQ, SPGQG, SPGQGT, SPGQGTQ, SPGQGTQS, SPGQGTQSE, or SPGQGTQSEN from the amino terminus of SEQ ID NO:1).

hIL10 Muteins

The present disclosure further provides homodimeric or heterodimeric hIL10 muteins comprising at least one hIL10 monomer variant described above.

In some embodiments, the present disclosure provides a heterodimeric hIL10 mutein. As used herein, the term hIL10 heterodimeric mutein is used to refer to a dimeric hIL10 mutein wherein the two hIL10 monomer subunits of the hIL10 mutein comprise different amino acid sequences and at least one of the two hIL10 monomer subunits of the heterodimeric hIL10 mutein comprises is an hIL10 monomer variant.

In one embodiment, the hIL10 heterodimeric mutein comprises: (a) first hIL10 monomer variant having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO: 1) and comprising at least one amino acid substitution selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C, optionally comprising one or more amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO: 1; and (b) a second hIL10 monomer variant having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1) and comprising at least one amino acid substitution selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C, optionally comprising one or more amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO: 1, wherein the first hIL10 monomer and the second hIL10 monomer are different.

In one embodiment, the hIL10 heterodimeric mutein comprises: (a) first hIL10 monomer variant comprising at least one amino acid substitution selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C, optionally comprising one or more amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO:1; and (b) a second hIL10 monomer variant comprising at least one amino acid substitution selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C, optionally comprising one or more amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO:1, wherein the amino acid sequence of the first hIL10 monomer and/or second hIL10 monomer has at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1), wherein the first hIL10 monomer and the second hIL10 monomer are different.

In one embodiment, the hIL10 heterodimeric mutein comprises: (a) first hIL10 monomer variant comprising one or more amino acid substitutions at positions selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C, optionally comprising one or more amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO:1; and (b) a wild-type hIL10 monomer.

In some embodiments, the hIL10 mutein is a homodimeric mutein. As used herein, the term homodimeric hIL10 mutein refers to an hIL10 mutein wherein each monomer subunit of the hIL10 mutein dimer is comprised of two identical hIL10 variant polypeptide monomers. In one embodiment, the present disclosure provides a homodimeric hIL10 mutein comprised of two hIL10 monomer variants wherein each hIL10 monomer variant has at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1) and comprises at least one amino acid substitution selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C, and optionally further comprises one or more amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO:1. In one embodiment, the present disclosure provides a homodimeric hIL10 mutein comprised of two hIL10 monomer variants comprising at least one amino acid substitution selected from the group consisting of H14C, H14F, H14P, H14W, H14G, N18R, N18K, N21C, N21D, N21E, M22D, M22S, M22T, M22W, D25P, D25Q, R32N, R32Q, R32G, R32C, R32P, R32F, R32Y, S93G, E96C, E96F, E96Y, E96W, and T100C, and optionally further comprises one or more amino acid substitutions selected from the group consisting of H14A, H14D, H14E, H14I, H14K, H14L, H14M, H14N, H14Q, H14R, H14S, H14T, H14Y, H14V, N18Y, N18F, N18A, N18D, N18E, N18L, N18V, N18S, N18T, N18I, N18V, N18M, N18H, N21A, N21R, N21Q, N21H, N21K, N21S, N21V, N21I, N21L, N21M, N21T, M22A, M22V, M22I, M22L, M22N, M22Q, R24E, R24D, R24N, R24Q, R24A, R24S, R24T, D25A, D25N, D25H, D25I, D25K, D25L, D25V, D28A, D28E, D28L, D28V, D28S, D28T, D28I, D28V, D28M, D28H, D28K, D28R, R32A, R32D, R32E, R32L, R32V, R32S, R32T, R32I, R32V, R32M, R32H, E74A, E74D, E74L, E74V, E74S, E74T, E74I, E74V, E74M, E74H, E74K, E74R, H90A, H90D, H90E, H90I, H90K, H90L, H90M, H90N, H90Q, H90R, H90S, H90T, H90Y, H90V, N92D, N92Q, N92E, N92H, N92K, N92S, N92V, N92I, N92L, N92M, N92T, N92A, S93E, S93A, S93R, S93N, S93D, S93Q, S93E, S93I, S93L, S93K, S93M, S93V, E96A, E96N, E96D, E96Q, E96H, E96K, E96S, T100D, T100V, T100E, T100A, T100R, T100N, T100Q, T100E, T100I, T100L, T100K, T100M, T100S, R104A, R104W, R104Y, R104F, R104H, R104D, R104E, R104N, R104Q, R104S, R104T, R104I, R104L, R104V, and R104M numbered in accordance with SEQ ID NO:1, wherein the amino acid sequence of each hIL10 monomer variant has at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1).

In some embodiments, the homodimeric or heterodimeric hIL10 muteins comprise a first and/or second hIL10 monomer variant comprising an amino acid sequence having at least 70% sequence identity (e.g., at least 70%, 75%, 80%, 85%, 90%, 91%, 92%, 93%, 94%, 95%, 96%, 97%, 98%, or 99% sequence identity) to a sequence comprising a deletion of one to 10 contiguous amino acids (e.g., a deletion of 1, 2, 3, 4, 5, 6, 7, 8, 9 or 10 contiguous amino acids) from the amino terminus of SEQ ID NO:1. In some embodiments, the homodimeric or heterodimeric hIL10 muteins comprise a first and/or second hIL10 monomer variant comprising an amino acid sequence having at least 70% sequence identity (e.g., at least 70%, 75%, 80%, 85%, 90%, 91%, 92%, 93%, 94%, 95%, 96%, 97%, 98%, or 99% sequence identity) to a sequence comprising a deletion of one to 10 contiguous amino acids (e.g., a deletion of 1, 2, 3, 4, 5, 6, 7, 8, 9 or 10 contiguous amino acids) from the amino acid sequence SPGQGTQSEN located at the amino terminus of SEQ ID NO:1 (e.g., a deletion of S, SP, SPG, SPGQ, SPGQG, SPGQGT, SPGQGTQ, SPGQGTQS, SPGQGTQSE, or SPGQGTQSEN from the amino terminus of SEQ ID NO:1).

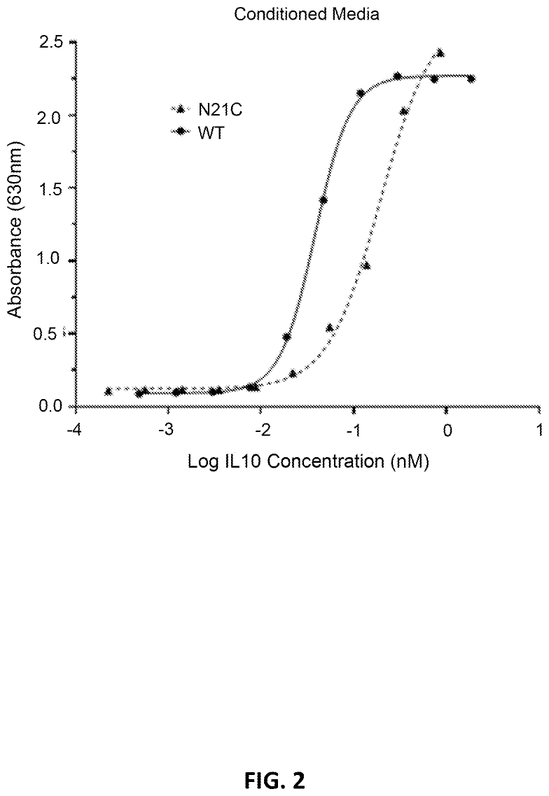

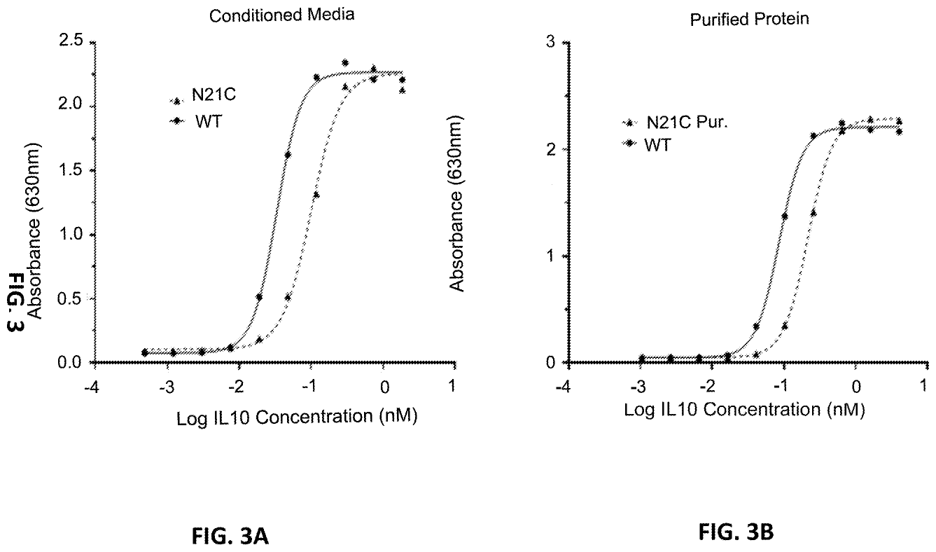

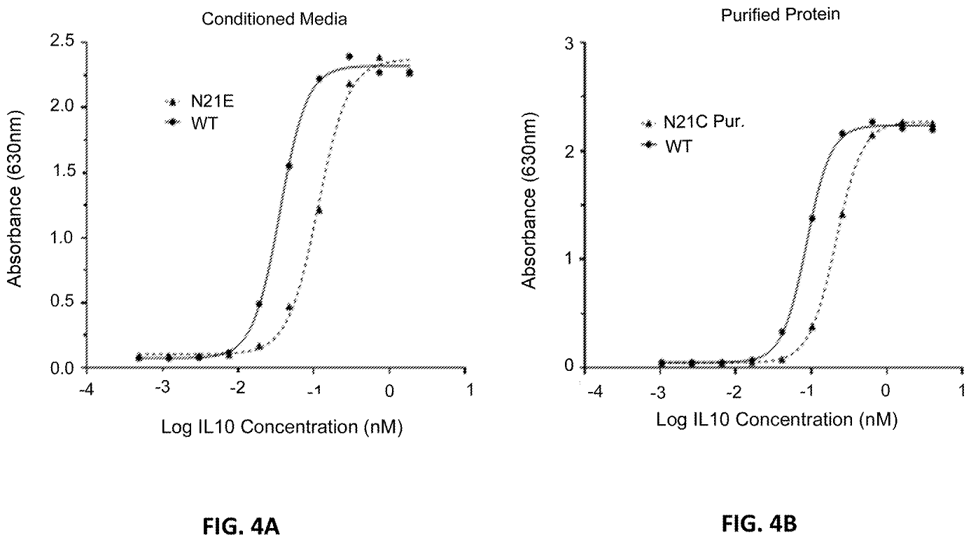

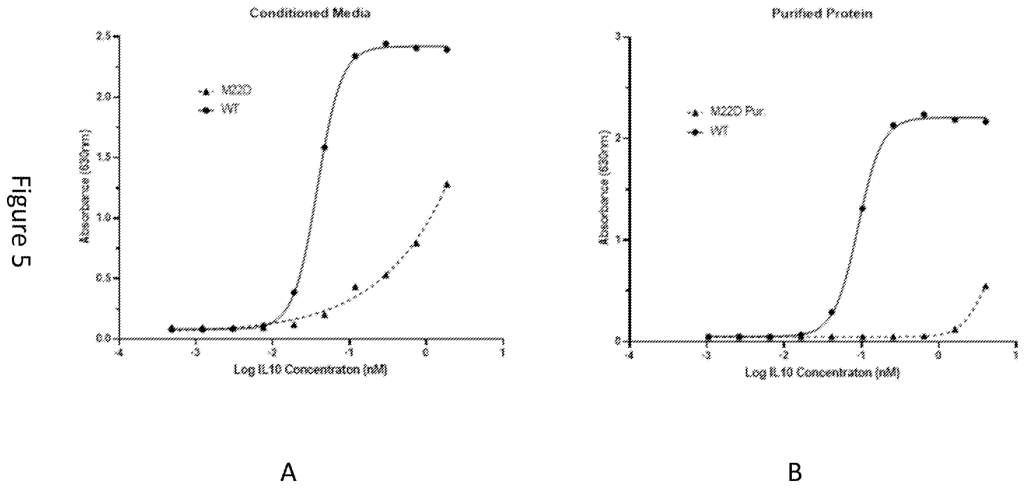

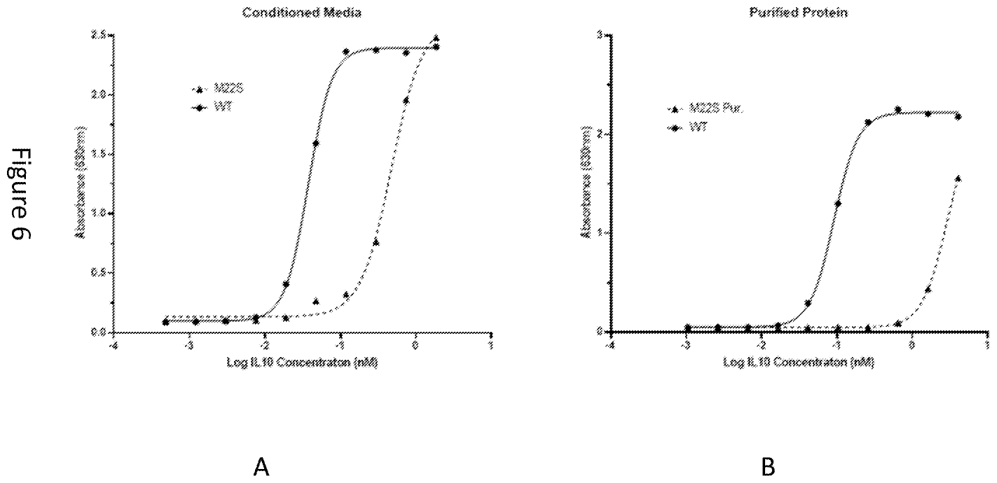

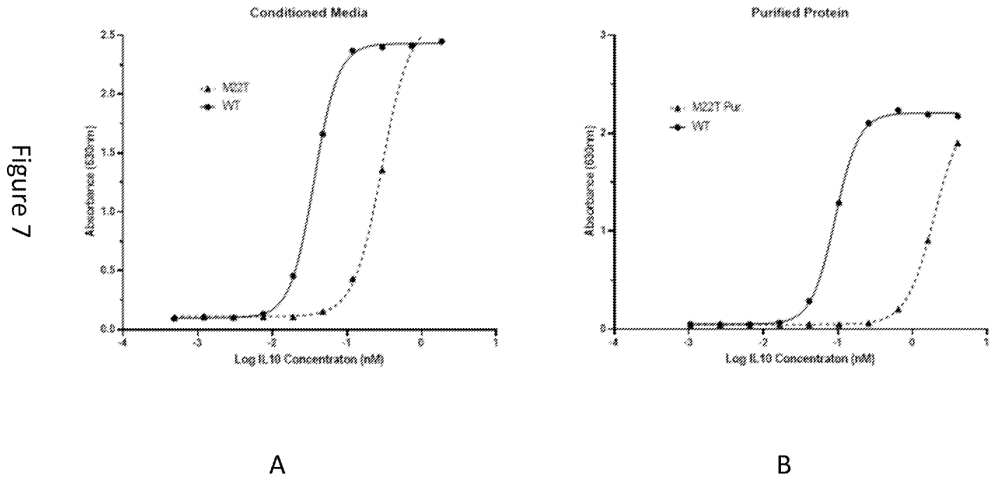

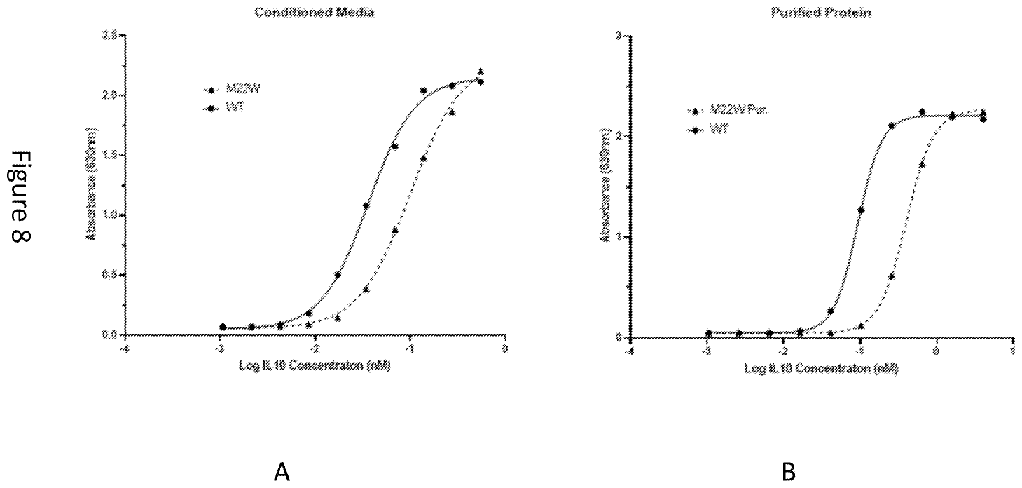

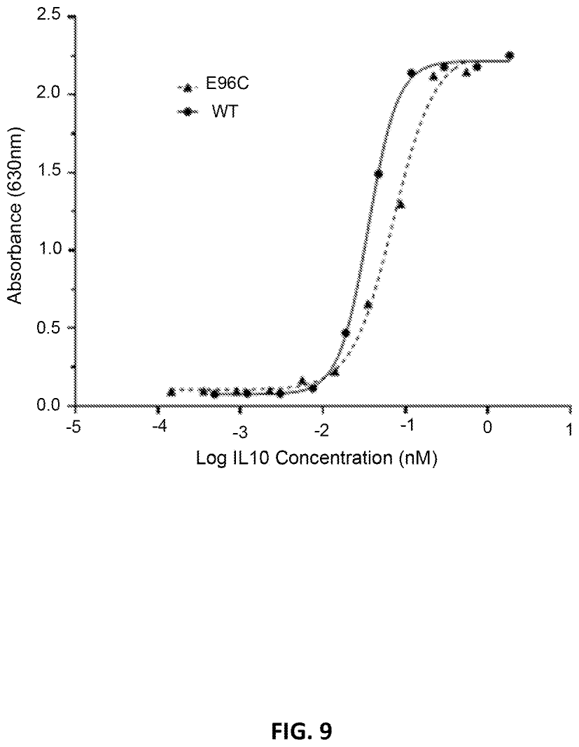

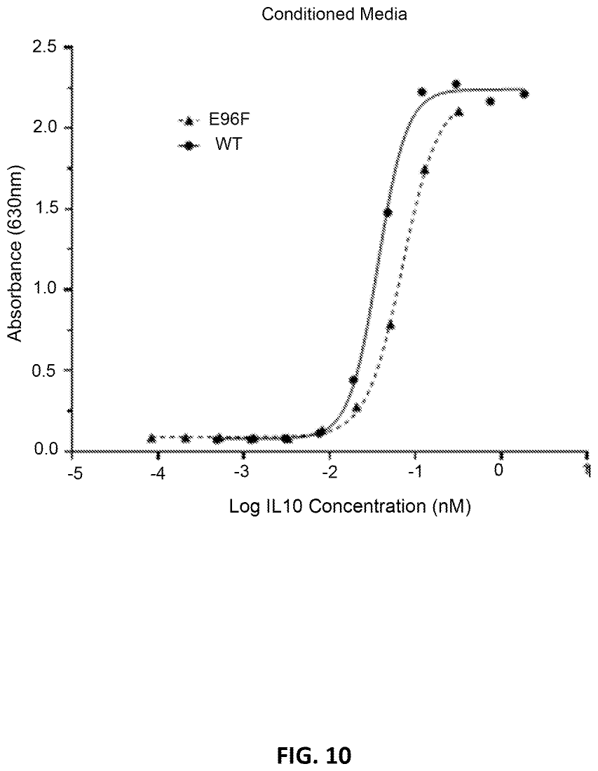

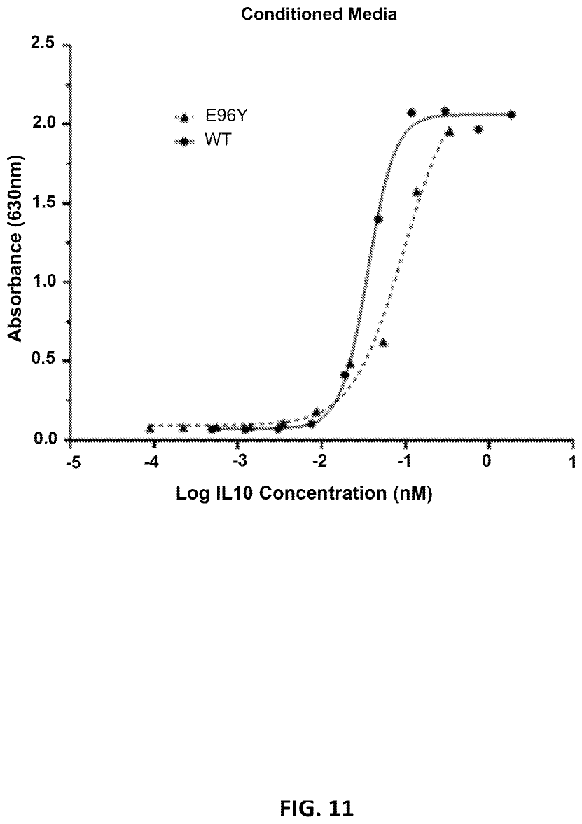

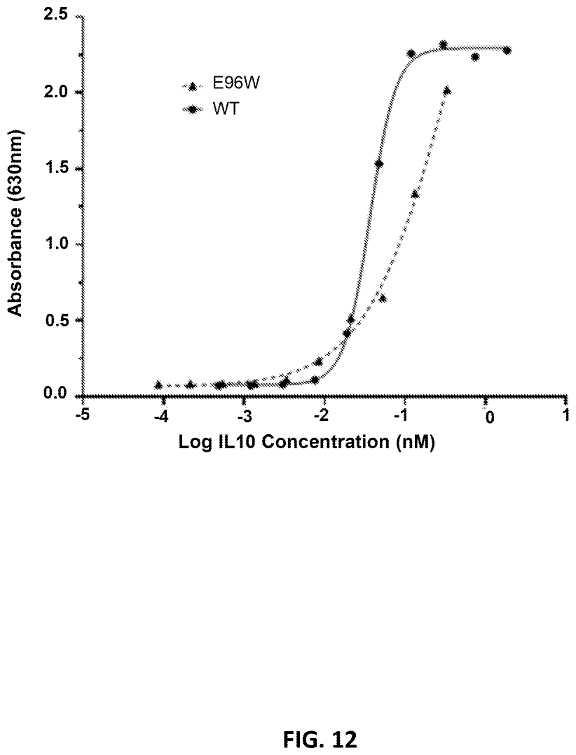

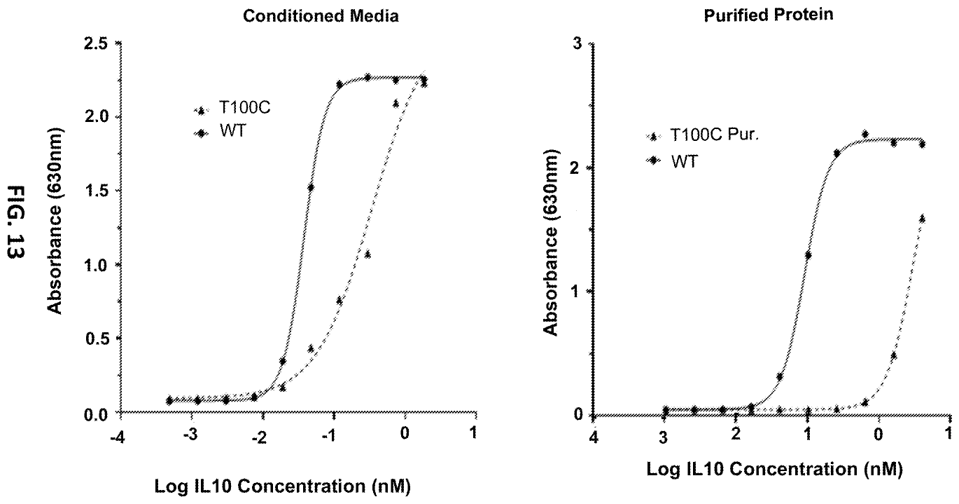

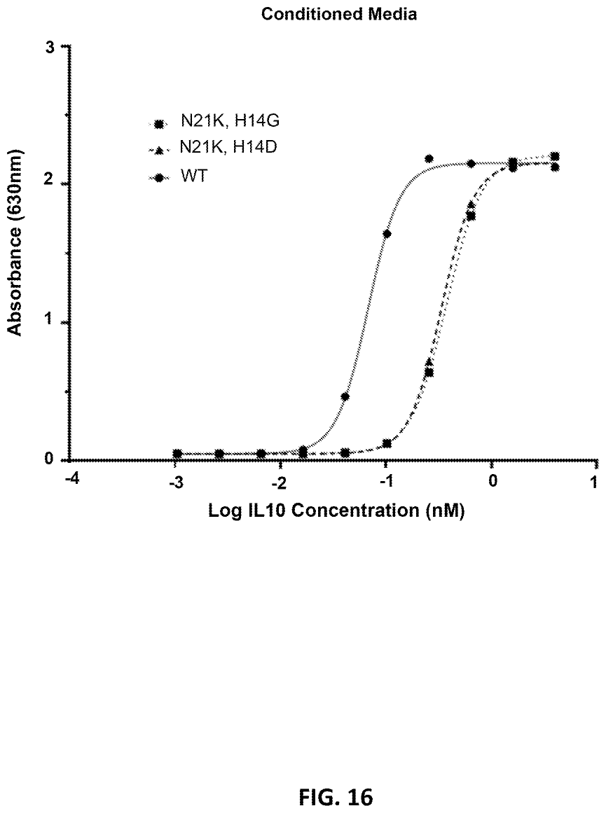

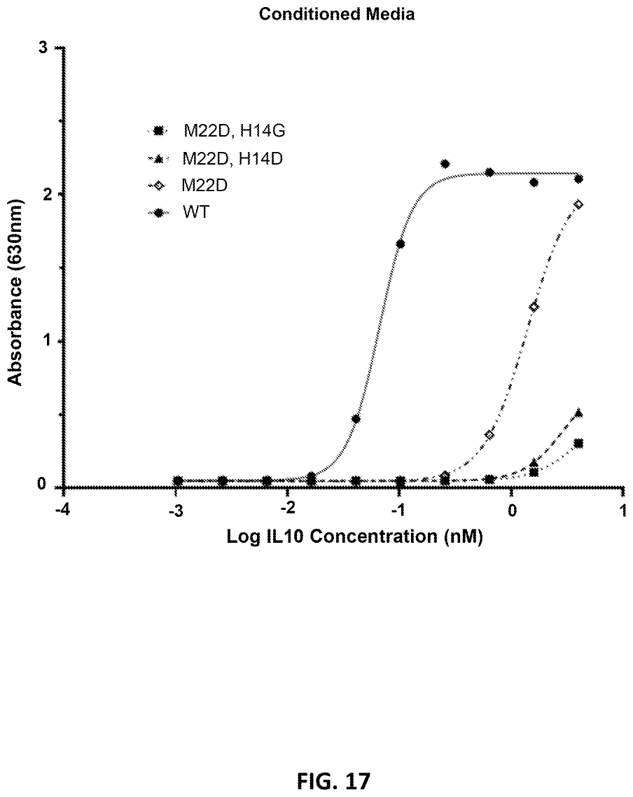

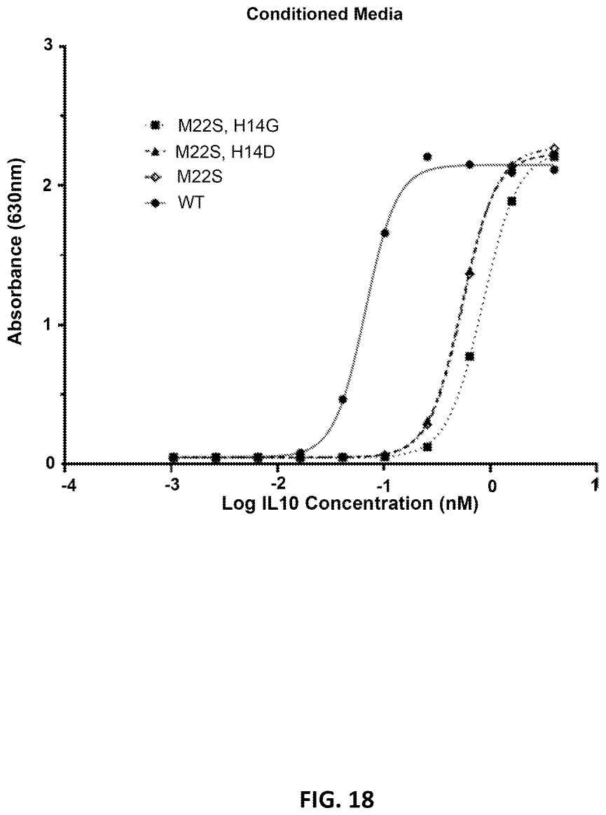

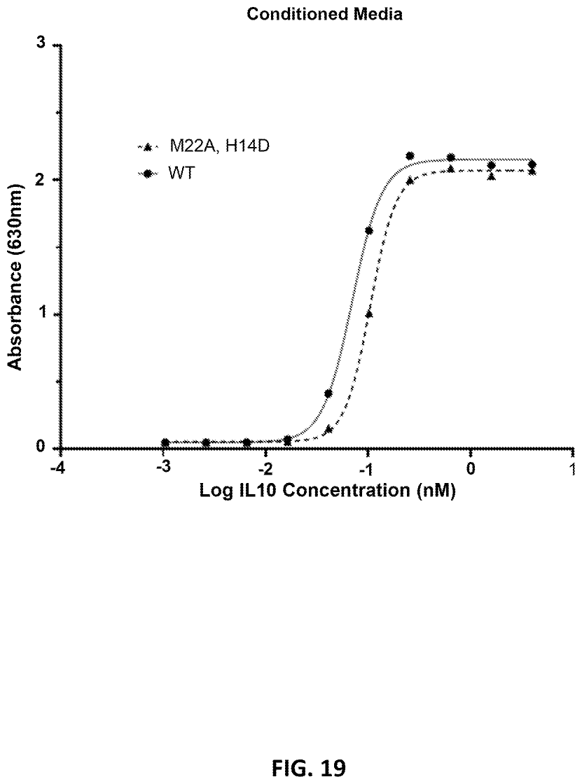

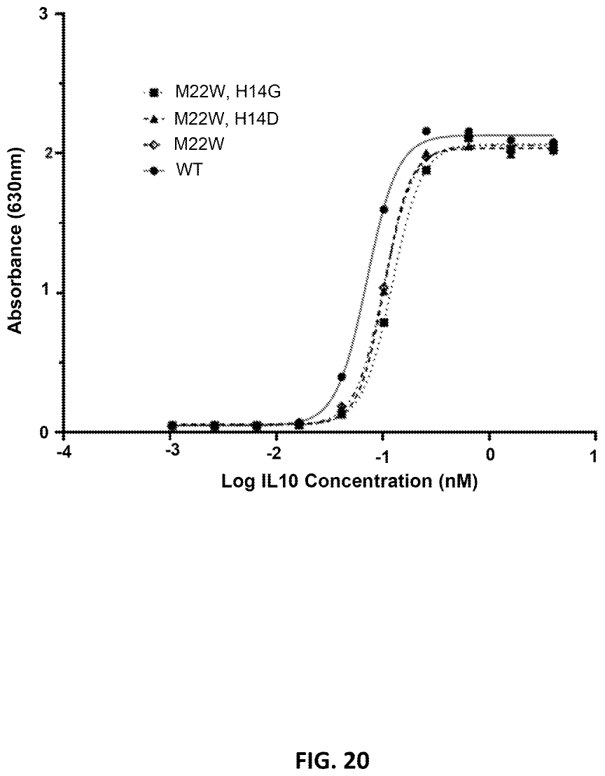

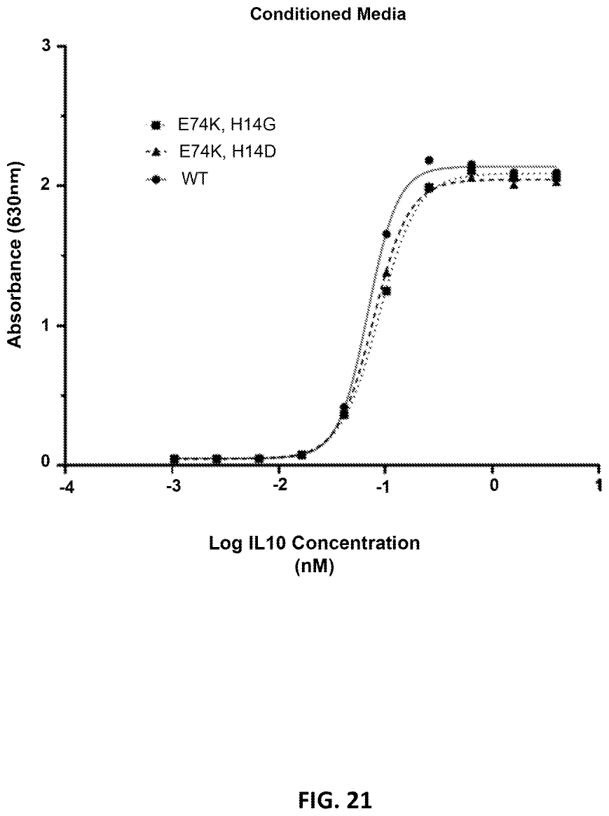

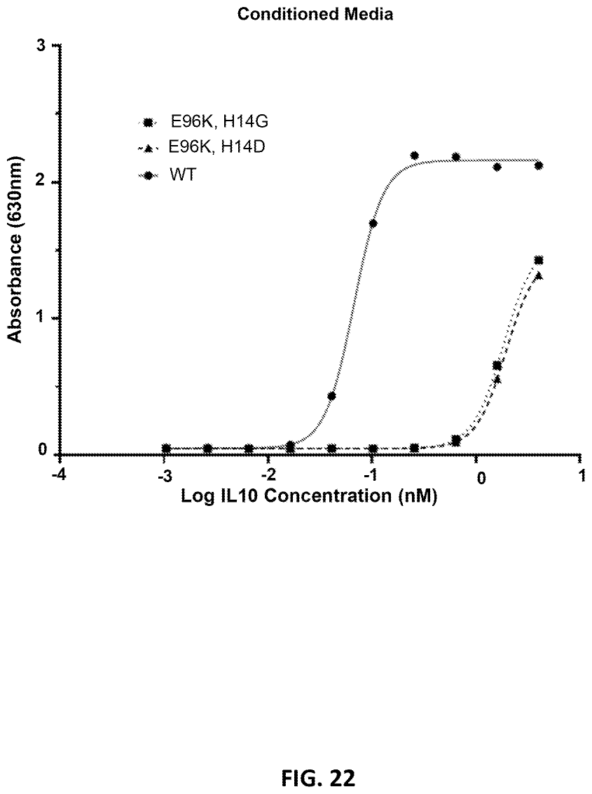

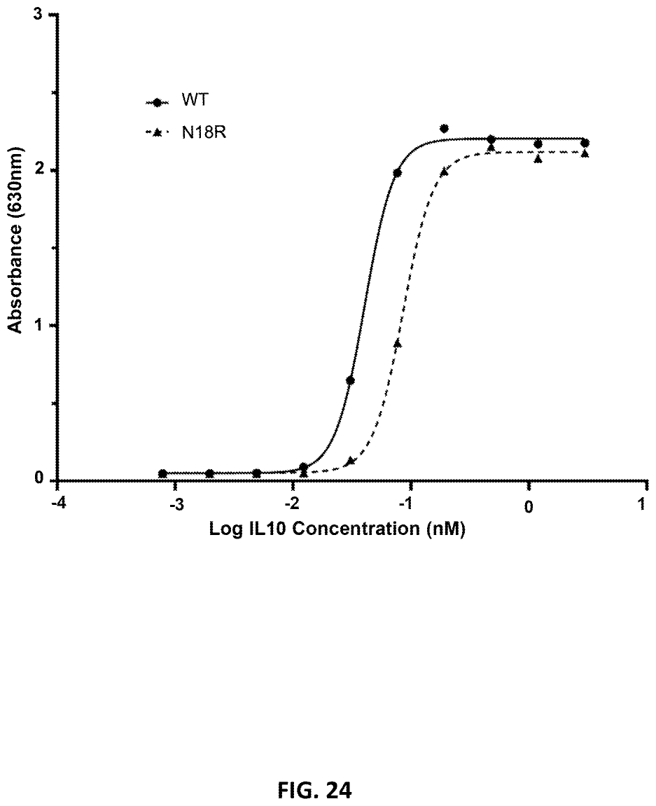

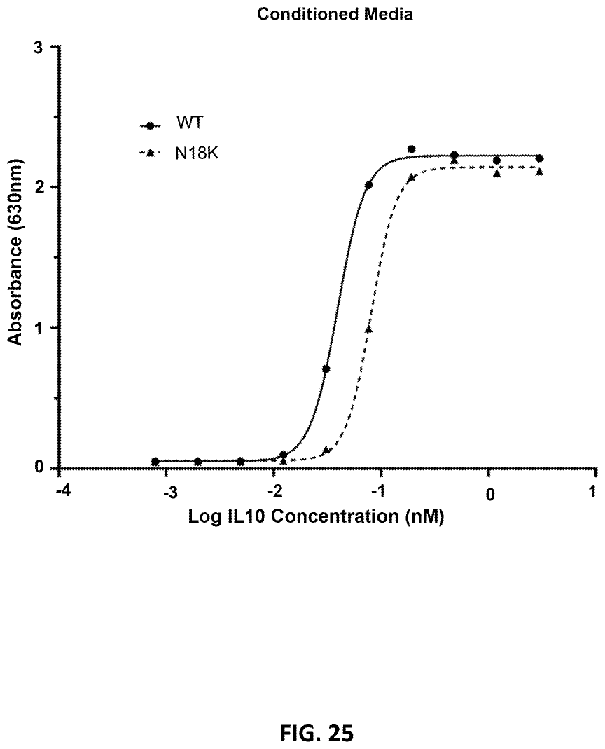

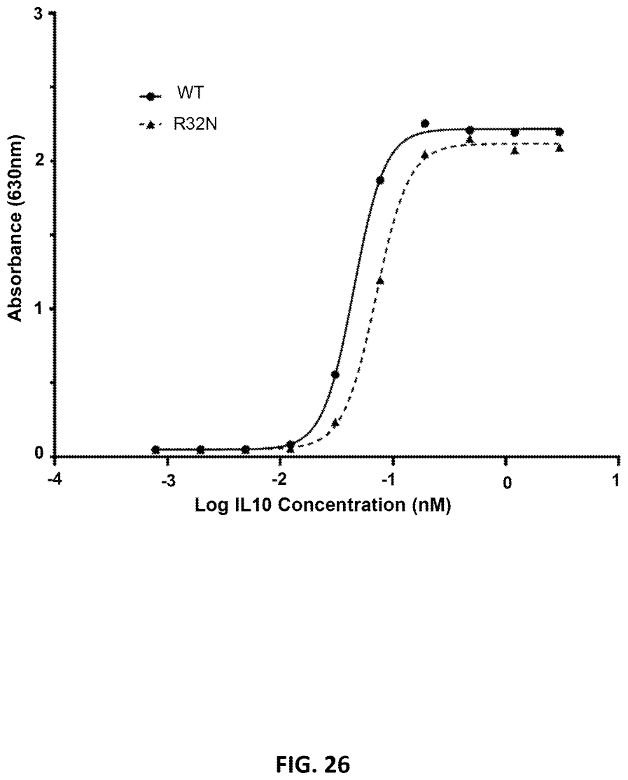

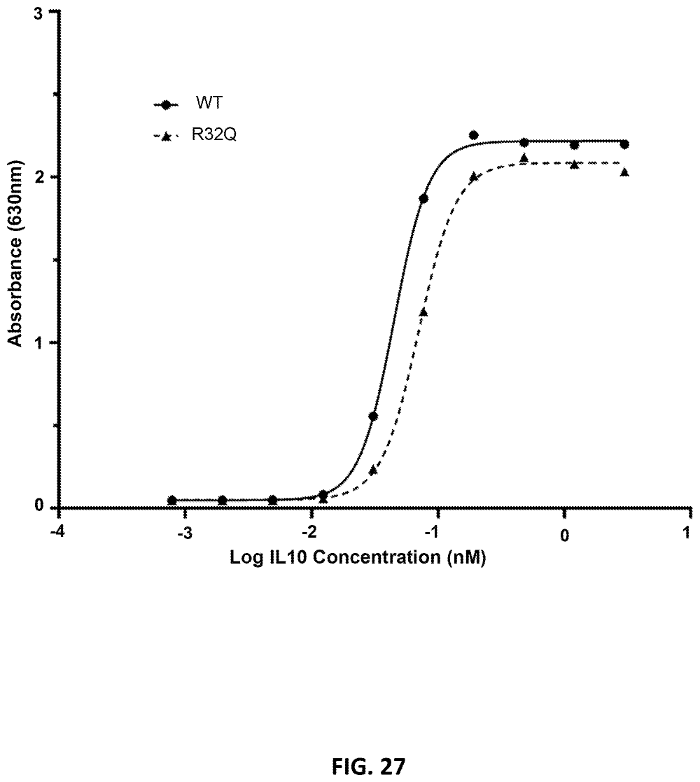

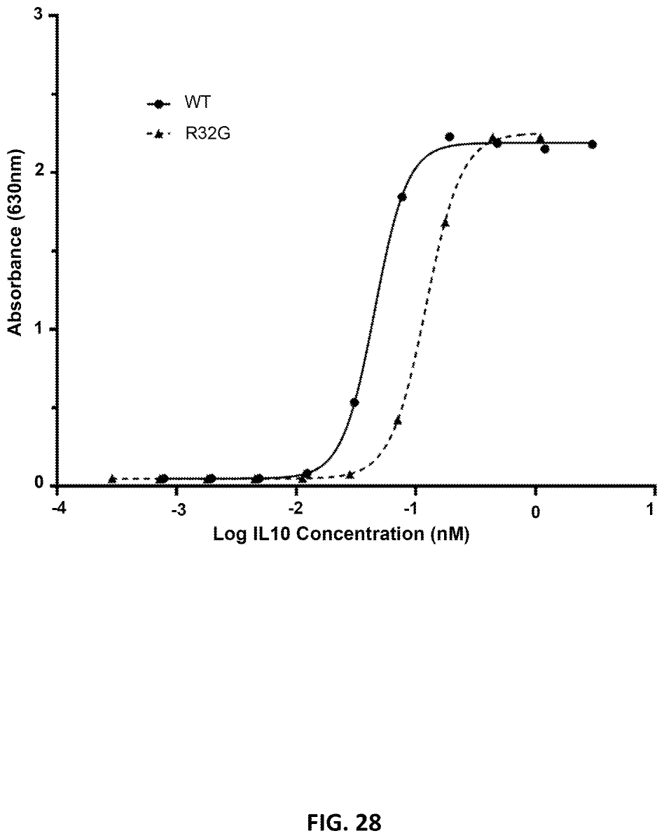

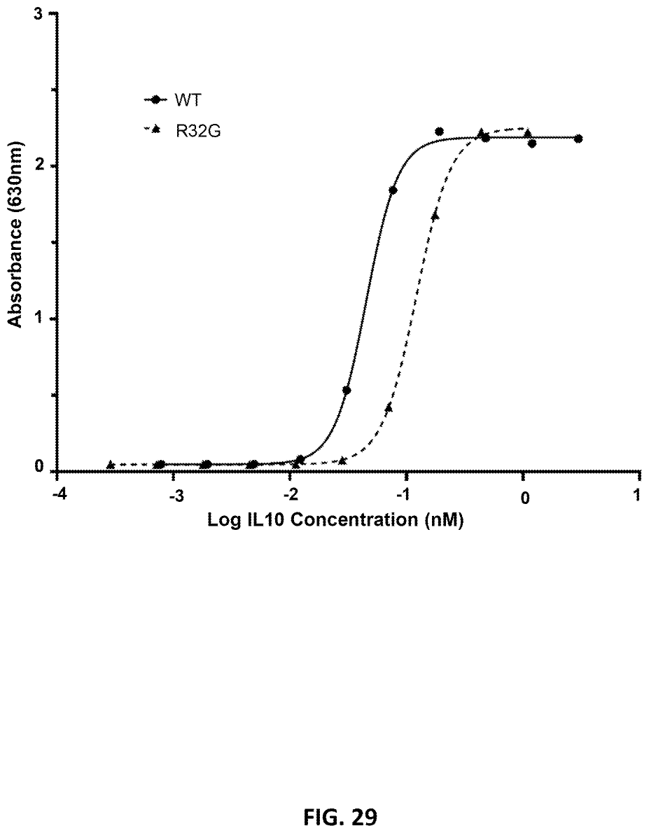

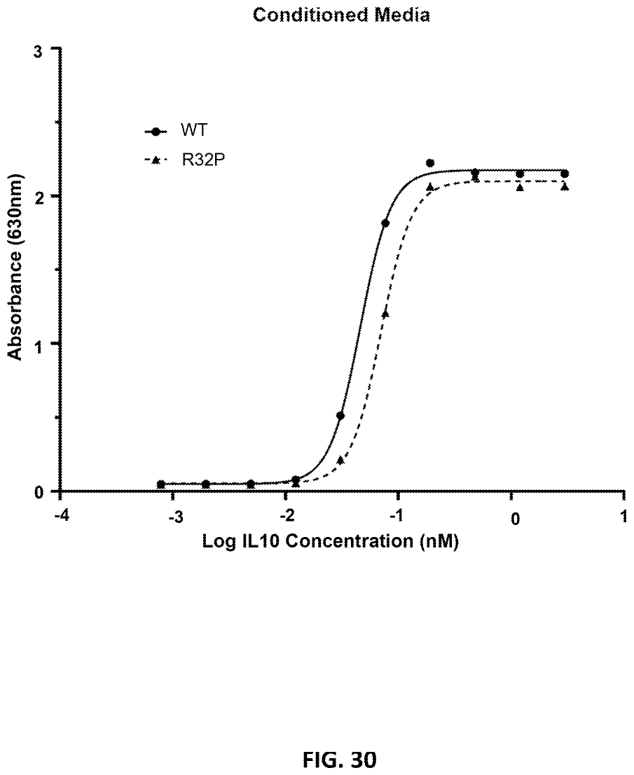

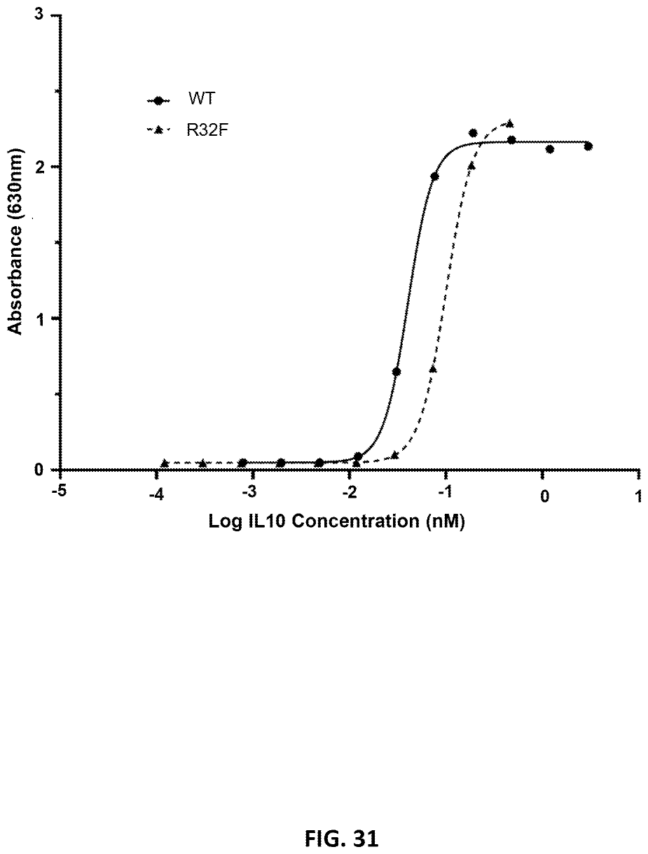

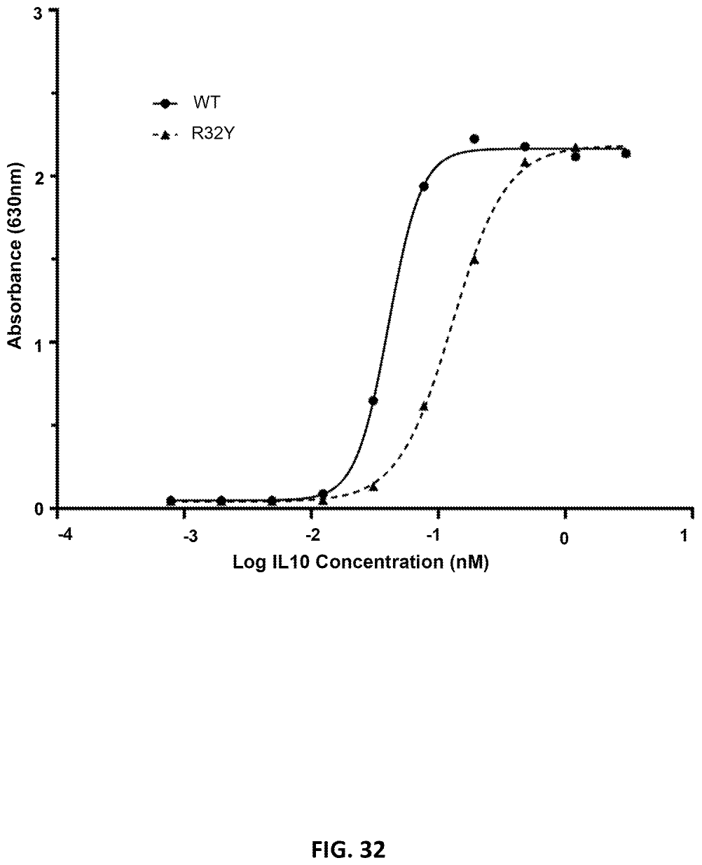

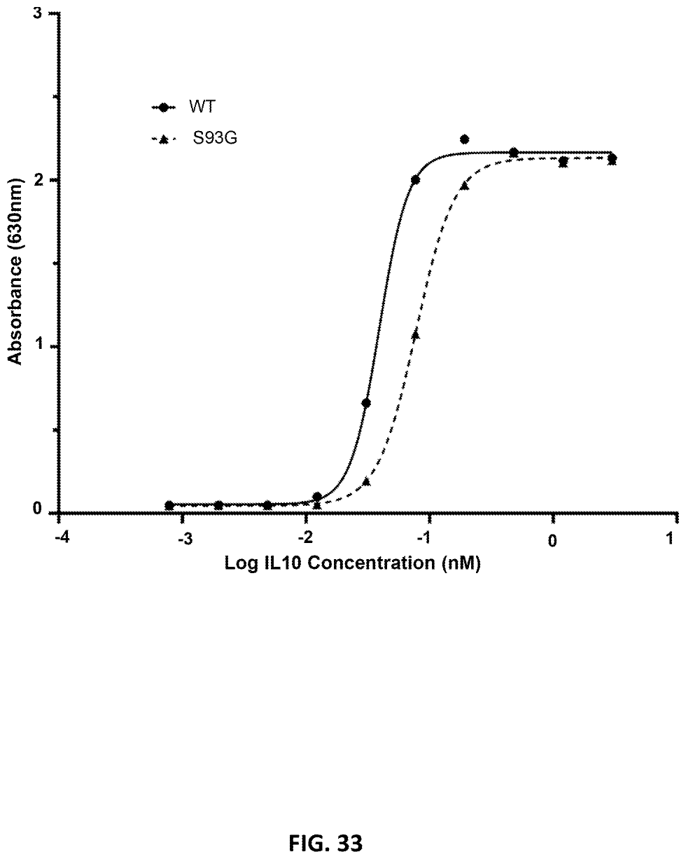

A selection of hIL10 muteins comprising hIL10 variant monomers were evaluated for hIL10 activity in a reporter assay wherein HEK293 cell line that are been modified to express the hIL10 receptor to provide STAT3 signaling which in upregulates expression and secretion of SEAP, a secreted truncated form of human placental alkaline phosphatase. The level of alkaline phosphatase activity thereby correlates with the level of STAT3 activation in the cell. The various hIL10 muteins comprising hIL10 variant monomers were evaluated at increasing concentrations with a wild-type hIL10 control in substantial accordance with the teaching of the Examples herein. The results of these experiments are provided in FIGS. 2 to 13, 15 to 22, and 24 to 33 of the attached drawings. As indicated, the hIL10 muteins comprising hIL10 variant monomers of the present disclosure demonstrated hIL10 activity in the reporter assay.

Modulated Affinity for hIL10Rb

In some embodiments, hIL10 monomer variants as described herein and hIL10 muteins comprising at least one such monomer has modulated binding affinity for the hIL10Rβ receptor subunit relative to a wt hIL10 monomer (SEQ ID NO:1) or wild-type hIL10, respectively. In some embodiments, hIL10 monomer variants as described herein and hIL10 muteins comprising at least one such monomer has reduced binding affinity for the hIL10Rβ receptor subunit relative to a wt hIL10 monomer (SEQ ID NO:1) or wild-type hIL10, respectively.

### Biased Activity

In some embodiments, the hIL10 muteins comprising hIL10 variant polypeptide monomers described herein are partial agonists of STAT3-mediated signaling (“STAT3 signaling”). In some embodiments, the hIL10 muteins comprising hIL10 variant polypeptide monomers described herein activate STAT3 signaling in some cell types, and result in decreased STAT3 signaling in other cell types. In some embodiments, the hIL10 muteins comprising hIL10 variant polypeptide monomers activate STAT3 signaling in myeloid cells, and produce decreased STAT3 signaling in lymphocytes. In some embodiments, the myeloid cell is a neutrophil, eosinophil, mast cell, basophil or monocyte. In some embodiments, the monocyte is a macrophage or a dendritic cell. In some embodiments, the macrophage is a Kupffer cell. In some embodiments, the lymphocyte is a CD8+ T cell, a CD4+ T cell, a B cell or an NK cell.

In some embodiments, the hIL10 muteins of the present disclosure have a pSTAT3 Eₘₐₓ of greater than 20%, greater than 30%, greater than 40%, greater than 50%, greater than 60%, or greater than 70% of the pSTAT3 Eₘₐₓ of wild-type hIL10 in myeloid cells. In some embodiments, an hIL10 mutein of the present disclosure exhibits decreased STAT3-mediated signaling in lymphocytes such as T cells, B cells or NK cells compared to wild-type hIL10. In some embodiments, an hIL10 mutein of the present disclosure has a pSTAT3 Eₘₐₓ in a lymphocyte less than 70%, less than 60%, less than 50%, less than 40%, or less than 30%, of the pSTAT3 Eₘₐₓ of a wild-type hIL10 lymphocyte. In some embodiments, dimers of the hIL10 muteins result in a pSTAT3 Eₘₐₓ in a lymphocyte less than 70% (e.g., less than 70%, less than 60%, less than 50%, less than 40%, or less than 30%) but greater than 20% of the pSTAT3 Eₘₐₓ of a wild-type or parental IL10 polypeptide in the lymphocyte. In some embodiments, the lymphocyte is selected from a CD8+ T cell, a CD4+ T cell, a B cell or an NK cell.

In some embodiments, the hIL10 muteins of the present disclosure provide greater activity on cells (e.g., STAT3 activity) which have higher surface expression of the hIL10Rβ receptor subunit relative to cells with that have lower surface expression of the hIL10Rβ receptor subunit. Cell types which are characterized as having high surface expression of the hIL10Rβ subunit include cells of myeloid origin, in particular activated myeloid cells. Cell types which are characterized as having low surface expression of the hIL10Rβ receptor subunit include lymphocytes such as CD8+ T cells, CD4+ T cells, B cells or NK cells. In some embodiments, the myeloid cell is selected from a myelocyte, granulocyte, (e.g. (neutrophil, eosinophil, or basophil), mast cell, or monocyte. In some embodiments, the monocyte is a macrophage or dendritic cell. In some embodiments, the macrophage is a Kupffer cell.

The hIL10 muteins of the present disclosure inhibit pro-inflammatory responses and/or STAT3-mediated signaling in a cell-type dependent manner, such that inflammatory macrophage activation is inhibited without substantially promoting the production of inflammatory cytokines such as interferon-γ by T cells. In some embodiments, an hIL10 mutein of the present disclosure retains the immunosuppressive functions of wild-type hIL10, such as inhibiting the production of inflammatory cytokines, while decreasing the immunostimulatory functions of wild-type hIL10, such as the production of IFN-gamma by CD8⁺ T cells.

For example, the hIL10 muteins of the present disclosure retain activity comparable to wild-type hIL10 to suppress myeloid cell activation (e.g., as evaluated by increased STAT3-mediated signaling in myeloid cells), but possess substantially reduced activation (e.g., as evaluated by decreased production of IFN-gamma) in PBMCs, T cells, B cells and NK cells.

In some embodiments, the the hIL10 muteins of the present disclosure are hIL10 partial agonists.

### Murine IL10

Mouse (or murine) IL10 (mIL10) is a non-covalently linked homodimeric protein comprising two identical mIL10 monomer subunits. Each mIL10 monomer is expressed as a 178 amino acid pre-protein comprising 18 amino acid signal sequence which is post-translationally removed to render a 160 amino acid mature protein. The canonical amino acid sequence of the mature mIL10 protein (UniProt Reference No. P18893) monomer without the signal sequence (corresponding to amino acids 19-178 of the pre-protein) is:

(SEQ ID NO: 3)
SRGQYSREDN NCTHEPVGQS HMLLELRTAF SQVKTFFQTK
DQLDNILLTD SLMQDFKGYL GCQALSEMIQ FYLVEVMPQA
EKHGPEIKEH LNSLGEKLKT LRMRLRRCHR FLPCENKSKA
VEQVKSDFNK LQDQGVYKAM NEFDIFINCI EAYMMIKMKS.

To facilitate evaluation of the activity of the hIL10 muteins of the present disclosure in murine in vivo models of human disease, the present disclosure further provides mIL10 surrogates of the hIL10 molecules of the present disclosure. The preparation of the murine IL10 surrogate may be accomplished by substitution of the corresponding human IL10 polypeptide substitution(s) into the mouse IL10 polypeptide in accordance with the following alignment of the mouse and human sequences:

| | | | |
|---|---|---|---|
| IL10_MOUSE | 1 | SRGQYSREDNNCTHFPVGQSHMLLELRTAFSQVKTFFQTKDQLDNILLTDSLMQDFKGYL | 60 |
| IL10_HUMAN | 1 | SPGQGTQSENSCTHFPGNLPNMLRDLRDAFSRVKTFPQMKDQLDNLLLKESLLEDFKGYL | 60 |
| | | * ** ::.:*.***** . :** :** ***:****** ******:**.:**::****** | |
| IL10_MOUSE | 61 | GCQALSEMIQFYLVEVMPQAEKEGPEIKEHLNSLGEKLKTLRMRLRRCHRFLPCENKSKA | 120 |
| IL10_HUMAN | 61 | GCQALSEMIQFYLEEVMPQAENQDPDIKAHVNSLGENLKTLRLRLRRCHRFLPCENKSKA | 120 |
| | | ************* *******::.*:** *:*****:*****:***************** | |
| IL10_MOUSE | 121 | VEQVKSDFNKLQDQGVYKAMNEFDIFINCIEAYMMIKMKS | 160 |
| IL10_HUMAN | 121 | VEQVKNAFNKLQEKGIYKAMSEFDIFINYIEAYMTMKIRN | 160 |
| | | *****. *****::*:****.******* ***** :*::. | |

mIL10 Monomer Variants:

In some embodiments, the mIL10 variant polypeptide monomer has at least 70% sequence identity to SEQ ID NO:3 (e.g., at least 70%, 75%, 80%, 85%, 90%, 91%, 92%, 93%, 94%, 95%, 96%, 97%, 98%, or 99% sequence identity to SEQ ID NO:3) and comprises one or more amino acid substitutions at a position corresponding to residues H14, G18, H21, M22, Q32, D25, S93, E96, and T100 of SEQ ID NO:3. In some embodiments, mIL10 monomer variant comprises one or more amino acid substitutions corresponding to residues H14, N18, N21, M22, D25, R32, S93, E96, and T100 of SEQ ID NO:1, wherein (a) the amino acid substitution at position H14 is selected from H14C, H14G, H14P, H14F, H14W; (b) the amino acid substitution at position N18 is selected from N18R and N18K; (c) the amino acid substitution at position H21 is selected from the group consisting of H21C, H21D, or H21E; (d) the amino acid substitution at position M22 is selected from the group consisting of M22D, M22S, M22T, or M22W; (e) the amino acid substitution at position R32 is selected from R32N, R32Q, R32G, R32C, R32P, R32F, and R32Y; (f) the amino acid substitution at position D25 is selected from the group consisting of D25P and D25Q; (g) the amino acid substitution at position S93 is S93G; (h) the amino acid substitution at position E96 is selected from the group consisting of E96C, E96F, E96Y; and (i) the amino acid substitution at position T100 is T100C.

The present disclosure provides mouse IL10 (mIL10) monomer variants having at least 70% sequence identity to SEQ ID NO:3 (e.g., at least 70%, 75%, 80%, 85%, 90%, 91%, 92%, 93%, 94%, 95%, 96%, 97%, 98%, or 99% sequence identity to SEQ ID NO:3) and comprising one or more amino substitutions at the amino acid residues selected from the group consisting of H14, G18, H21, M22, E25, Q32, H90, N92, S93, E96, K99 and T100 numbered in accordance with SEQ ID NO:3.

### Modifications to Provide Additional Functions

In some embodiments, the hIL10 mutein may comprise a functional domain of a chimeric polypeptide. HIL10 mutein fusion proteins of the present disclosure may be readily produced by recombinant DNA methodology by techniques known in the art by constructing a recombinant vector comprising a nucleic acid sequence comprising a nucleic acid sequence encoding the hIL10 mutein in frame with a nucleic acid sequence encoding the fusion partner either at the N-terminus or C-terminus of the hIL10 muteins, the sequence optionally further comprising a nucleic acid sequence in frame encoding a linker or spacer polypeptide.

In other embodiments, the hIL10 mutein can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the binding molecule further comprises a C-terminal c-myc epitope tag.

In some embodiments, the hIL10 mutein is conjugated to a molecule (“targeting domain”) to facilitate selective binding to particular cell type or tissue expressing a cell surface molecule that specifically binds to such targeting domain, optionally incorporating a linker molecule of from 1-40 (alternatively 2-20, alternatively 5-20, alternatively 10-20) amino acids between the hIL10 mutein sequence and the sequence of the targeting domain of the fusion protein.

In other embodiments, a chimeric polypeptide including a hIL10 mutein and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

Association with Carrier Molecules to Increase Duration of Action

The hIL10 muteins described herein can be modified to provide for an extended lifetime in vivo and/or extended duration of action in a subject. In some embodiments, one or both of the hIL10 monomers of the hIL10 mutein are conjugated to carrier molecules to provide desired pharmacological properties such as an extended half-life. In some embodiments, the one or both of the hIL10 monomers of the hIL10 mutein are covalently linked to the Fc domain of an IgG, albumin, water soluble polymers, or other molecules to extend its half-life, e.g. glycosylation, acylation and the like as known in the art. In some embodiments, the hIL10 variant monomer modified to provide an extended duration of action in a mammalian subject has a half-life in a mammalian of greater than 4 hours, alternatively greater than 5 hours, alternatively greater than 6 hours, alternatively greater than 7 hours, alternatively greater than 8 hours, alternatively greater than 9 hours, alternatively greater than 10 hours, alternatively greater than 12 hours, alternatively greater than 18 hours, alternatively greater than 24 hours, alternatively greater than 2 days, alternatively greater than 3 days, alternatively greater than 4 days, alternatively greater than 5 days, alternatively greater than 6 days, alternatively greater than 7 days, alternatively greater than 10 days, alternatively greater than 14 days, alternatively greater than 21 days, or alternatively greater than 30 days.

Modifications of the hIL10 variant monomer to provide an extended duration of action in a mammalian subject include (but are not limited to);
conjugation of the hIL10 variant monomer to one or more protein carrier molecules,
conjugation of the hIL10 variant monomer to protein carrier molecules, optionally in the form of a fusion protein with additional polypeptide sequences (e.g, hIL10 variant monomer-Fc fusions) and
conjugation to polymers, (e.g. water soluble polymers to provide a PEGylated IL10 polypeptide).

It should be noted that the more than one type of modification that provides for an extended duration of action in a mammalian subject may be employed with respect to a given hIL10 variant monomer. For example, a hIL10 variant monomer of the present disclosure may comprise both amino acid substitutions that provide for an extended duration of action as well as conjugation to a carrier molecule such as a polyethylene glycol (PEG) molecule.

### Protein Carrier Molecules:

Examples of protein carrier molecules which may be covalently attached to the hIL10 variant monomer to provide an extended duration of action in vivo include, but are not limited to albumins, antibodies and antibody fragments such and Fc domains of IgG molecules

### Fc Fusions:

In some embodiments, one hIL10 variant monomer is conjugated to each of the Fc domains to provide an IL10/Fc mutein. The Fc fusion format is particularly useful in the construction of heterodimeric hIL10 muteins as it provides for a 1:1 ratio of each species in the final product. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The “Fc region” useful in the preparation of Fc fusions can be a naturally occurring or synthetic polypeptide that is homologous to an IgG C-terminal domain produced by digestion of IgG with papain. IgG Fc has a molecular weight of approximately 50 kDa. The binding molecule described herein can be conjugated to the entire Fc region, or a smaller portion that retains the ability to extend the circulating half-life of a chimeric polypeptide of which it is a part. In addition, full-length or fragmented Fc regions can be variants of the wild-type molecule. In a typical presentation, each monomer of the dimeric Fc can carry a heterologous polypeptide, the heterologous polypeptides being the same or different.

As indicated, the linkage of the hIL10 variant monomer to the Fc subunit may incorporate a linker molecule between the hIL10 variant monomer and Fc subunit. In some embodiments, the hIL10 variant monomer is expressed as a fusion protein with the Fc domain incorporating an amino acid sequence of a hinge region of an IgG antibody. The Fc domains engineered in accordance with the foregoing may be derived from IgG1, IgG2, IgG3 and IgG4 mammalian IgG species. In some embodiments, the Fe domains may be derived from human IgG1, IgG2, IgG3 and IgG4 IgG species. In some embodiments, the hinge region is the hinge region of an IgG1. In one particular embodiment, the hIL10 variant monomer is linked to an Fc domain using an human IgG1 hinge domain.

In some embodiments, when the hIL10 variant is provided as an Fc fusion, the hIL10 variant comprises: (a) a fusion protein comprising a first hIL10 polypeptide monomer and a first Fc monomer (“Fc1”), optionally comprising a first linker (“L1”) between the first IL10 monomer sequence and Fc1, and (b) a fusion protein comprising a second hIL10 polypeptide monomer and a second Fc monomer (“Fc2”), optionally comprising a second linker (“L1”) between the second IL10 monomer sequence and the Fc2, wherein one or more of the hIL10 polypeptide monomers is an hIL10 monomer variant comprising one or more amino substitutions at the amino acid residues selected from the group consisting of H14, N18, N21, M22, R32, H90, N92, S93, E96, K99 and T100. In some embodiments, the linker is a chemical linker. Examples of chemical linkers include aryl acetylene, ethylene glycol oligomers containing 2-10 monomer units, diamines, diacids, amino acids, or combinations thereof. In some embodiments, the linker is a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids). Glycine and glycine-serine polymers are relatively unstructured, and therefore may serve as a neutral tether between components. Examples of glycine polymers include (G)n, glycine-alanine polymers, alanine-serine polymers, glycine-serine polymers (for example, (GmSo)n, (GSGGS)n, (GmSoGm)n, (GmSoGmSoGm)n, (GSGGSm)n, (GSGSmG)n and (GGGSm)n, and combinations thereof, where m, n, and o are each independently selected from an integer of at least 1 to 20, e.g., 1-18, 216, 3-14, 4-12, 5-10, 1, 2, 3, 4, 5, 6, 7, 8, 9, or 10), and other flexible linkers.

In some embodiments, the Fe domains of the IL10/Fc mutein may be engineered to possess a “knob-into-hole modification” facilitate heterodimerization. In one embodiment, Fc1 and Fc2 are modified to promote heterodimerization by the employment of the “knob-into-hole” (abbreviated KiH) modification as exemplified herein. The KiH modification comprises one or more amino acid substitutions in a first Fc monomer (e.g. Fc1) that create a bulky “knob” domain on a first Fe and one or more amino acid substitutions on a second Fc monomer (e.g. Fc2) that create a complementary pocket or “hole” to receive the “knob” of the first Fc monomer.

A variety of amino acid substitutions have been established for the creation of complementary knob and hole Fc monomers. See, e.g. Ridgway, et al (1996) Protein Engineering 9(7):617-921; Atwell, et al (1997) J. Mol. Biol. 270:26-35; Carter, et al. U.S. Pat. No. 5,807,706 issued Sep. 15, 1998; Carter, et al U.S. Pat. No. 7,695,936 issued Apr. 13, 2010; Zhao et al. “A new approach to produce IgG4-like bispecific antibodies,” Scientific Reports 11: 18630 (2021); Cao et al. “Characterization and Monitoring of a Novel Light-heavy-light Chain Mispair in a Therapeutic Bispecific Antibody,” and Liu et al. “Fc Engineering for Developing Therapeutic Bispecific Antibodies and Novel Scaffolds”. Frontiers in Immunology. 8: 38. doi:10.3389/fimmu.2017.00038 (2017).

In some embodiments, the Fc domain comprises two Fc monomers wherein the CH3 domain of a first Fc monomer wherein the threonine at (EU numbering) position 366 is modified with a bulky residue (e.g. a T366W) create a “knob” and the substitution, and a second Fc monomer comprising one or more substitutions in complementary residues of the CH3 domain of the second Fc monomer to create a pocket or “hole” to receive the bulky residue, for example by amino acid substitutions such as T366S, L368A, and/or Y407V.

In one embodiment, the Fc domain of the first IL10/Fc mutein is a “knob” modified Fc monomer comprising the amino acid substitution T366W and the Fc domain of the second IL10/Fc mutein is a “hole” modified Fc comprising the set of amino acid substitutions T366S/L368A/Y407V. Alternatively, the Fc domain of the first IL10/Fc mutein is a “hole” modified Fc monomer comprising the set of amino acid substitutions T366S/L368A/Y407V and the Fc domain of the second IL10/Fc mutein is a “knob” modified Fc monomer comprising the amino acid substitution T366W.

In some embodiments, the Fc domains of the IL10/Fc mutein are covalently linked via one or more, optionally two or more optionally three or more disulfide bonds, optionally four or more disulfide bonds between the side chains of the following groups of cystine pairs: (a) C96 of a first Fc monomer and the and C199 of a second Fc monomer; (b) between C226 of a first Fc monomer and the C226 of a second Fc monomer, (c) between C229 of a first Fc monomer and the C229 of a second Fc monomer; and (d) between S354C of a first Fc domain comprising a S354C amino acid substitution and Y349C of a second Fc domain comprising a Y349C amino acid substitution.

In some embodiments, the Fe domains of the IL10/Fc mutein are derived from hIgG4 domains of the heterodimeric hIL10 mutein are derived from hIgG4, heterodimerization of the first and second Fe domains by the introduction of the mutations K370E, K409W and E357N, D399V, F405T (EU numbering) in the complementary Fc sequences that comprise the heterodimeric Fc domain.

In addition to the modifications to promote heterodimerization of the first and second Fc monomers, the first and second Fc monomers may optionally provide additional amino acid modifications that mitigate effector function or eliminate the glycosylation site at N297 such as N297Q.

In some embodiments the amino acid sequence of the first and/or second Fc monomers of the IL10/Fc mutein are modified are modified to reduce effector function. In some embodiments, the Fc domain may be modified to substantially reduce binding to Fc receptors (FcyR and FcR) which reduces or abolishes antibody directed cytotoxicity (ADCC) effector function. Modification of Fe domains to reduce effector function are well known in the art. See, e.g., Wang, et al. (2018) IgG Fc engineering to modulate antibody effector functions, Protein Cell 9(1):63-73. For example, mutation of the lysine residue at position 235 (EU numbering) from leucine (L) to glutamic acid (E) is known to reduce effector function by reducing FcgR and C1q binding. Alegre, et al. (1992) J. Immunology 148:3461-3468. Additionally, substitution of the two leucine (L) residues at positions 234 and 235 (EU numbering) in the IgG1 hinge region with alanine (A), i.e., L234A and L235A, results in decreased complement dependent cytotoxicity (CDC) and antibody dependent cellular cytotoxicity (ADCC). Hezereh et al., (2001) J. Virol 75(24):12161-68. Furthermore, mutation of the proline at position 329 (EU numbering) to alanine (P329A) or glycine, (P329G) mitigates effector function and may be combined with the L234A and L235A substitutions. In some embodiments, the Fe domains may comprise the amino acid substitutions L234A/L235A/P329A (EU numbering) referred to as the “LALAPA” substitutions or L234A/L235A/P329G (EU numbering) referred to as the “LALAPG” substitutions. In some embodiments, the Fe domains may comprises the amino acid substitutions E233P/L234V/L235A/ΔG237 (referred to in the scientific literature as the PVAdelG mutation).

In some embodiments, the Fe domains of the IL10/Fc mutein are derived from hIgG4. In such instances where the Fe domains of the IL10/Fc mutein are derived from hIgG4, attenuation of effector function may be achieve by introduction of the S228P and/or the L235E mutations (EU numbering).

In some embodiments the amino acid sequence of the first and second Fc monomers modified to promote heterodimerization may be further modified to incorporate amino acid substitutions which extend the duration of action of the molecule and prevent clearance. In some embodiments, such modifications to the Fc monomer include the amino acid substitutions M428L and N434S (EU numbering) referred to as the “LS” modification. The LS modification may optionally be combined with amino acid substitutions to reduce effector function and provide for disulfide bonds between the Fc domains.

In some embodiments the amino acid sequence of the Fc1 and/or Fc2 monomers modified to promote heterodimerization may be further modified to eliminate N-linked or 0-linked glycosylation sites. Aglycosylated variants of Fc domains, particularly of the IgG1 subclass are known to be poor mediators of effector function. Jefferies et al. 1998, Immol. Rev., vol. 163, 50-76). It has been shown that glycosylation at position 297 (EU numbering) contributes to effector function. Edelman, et al (1969) PNAS (USA) 63:78-85. In some embodiments, the Fc domains of the compositions of the present disclosure comprise one or modifications to eliminate N- or O linked glycosylation sites. Examples of modifications at N297 to eliminate glycosylation sites in the Fc domain include the amino acid substitutions N297Q and N297G.

In some embodiments, a hIL10/Fc mutein may be further modified to extend its duration of action in vivo. In some embodiments, conjugation of the PEG moiety may be accomplished via a sulfhydryl (—SH) group of a cysteine residue. In some embodiments, the PEGylation of the homodimeric or heterodimeric hIL10/Fc muteins is provided at one or both of the naturally occurring cysteine residues at position 220 (C220, EU Numbering) of the upper hinge region of the hIL10/Fc muteins. In some embodiments, the PEGylation of homodimeric or heterodimeric hIL10/Fc muteins is provided at one or both of the naturally occurring cysteine residues at position 220 (C220, EU Numbering) of the upper hinge region of the Fc domains of the homodimeric or heterodimeric hIL20/Fc muteins. In preparing PEGylated heterodimeric or homodimeric hIL12Fc muteins or PEGylated heterodimeric hIL23Fc muteins where conjugation of the PEG molecule is provided at position C220, the above referenced C220S modification of the upper

### Albumin Carrier Molecules

In some embodiments, a hIL10 variant monomer is conjugated to an albumin molecule (e.g., human serum albumin) which is known in the art to facilitate extended exposure in vivo. In some embodiments, the hIL10 variant monomer is conjugated to albumin via chemical linkage or expressed as a fusion protein with an albumin molecule (referred to herein as a “hIL10 variant monomer albumin fusion”). The term “albumin” as used in the context hIL10 variant monomer-albumin fusions includes albumins such as human serum albumin (HSA), cyno serum albumin, and bovine serum albumin (BSA). In some embodiments, the HSA comprises a C34S or K573P amino acid substitution relative to the wild-type HSA sequence. According to the present disclosure, albumin can be conjugated to a hIL10 variant monomer at the carboxyl terminus, the amino terminus, both the carboxyl and amino termini, and internally (see, e.g., U.S. Pat. Nos. 5,876,969 and 7,056,701). In the HAS-hIL10 variant monomer contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising a hIL10 variant monomer fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. As an alternative to chemical linkage between the hIL10 variant monomer and the albumin molecule, the hIL10 variant monomer—albumin complex may be provided as a fusion protein comprising an albumin polypeptide sequence and a hIL10 variant monomer recombinantly expressed in a host cell as a single polypeptide chain, optionally comprising a linker molecule between the albumin and hIL10 variant monomer. Such fusion proteins may be readily prepared through recombinant technology to those of ordinary skill in the art. Nucleic acid sequences encoding such fusion proteins may be ordered from any of a variety of commercial sources. The nucleic acid sequence encoding the fusion protein is incorporated into an expression vector operably linked to one or more expression control elements, the vector introduced into a suitable host cell and the fusion protein solated from the host cell culture by techniques well known in the art.

### Polymeric Carriers

In some embodiments, extended in vivo duration of action of the hIL10 variant monomer or hIL10 mutein may be achieved by conjugation to one or more polymeric carrier molecules such as XTEN polymers or water soluble polymers.

### XTEN Conjugates

The hIL10 variant monomer or hIL10 mutein comprising such variant monomer may further comprise an XTEN polymer. The XTEN polymer conjugated (either chemically or as a fusion protein) to an hIL10 variant monomer or hIL10 mutein comprising such variant monomer provides extended duration akin to PEGylation and may be produced as a recombinant fusion protein in E. coli. XTEN polymers suitable for use in conjunction with the hIL10 variant monomer or hIL10 mutein comprising such variant monomer of the present disclosure are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease-Activatable Killin-Based Cytostatic” PLOS ONE DOI:10.1371/journal.pone.0157193 Jun. 13, 2016. The XTEN polymer may fusion protein may incorporate a protease sensitive cleavage site between the XTEN polypeptide and the hIL2 mutein such as an MMP-2 cleavage site.

### Water Soluble Polymers

In some embodiments, the hIL10 variant monomer can be conjugated to one or more water-soluble polymers. Examples of water soluble polymers useful in the practice of the present disclosure include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol,), polysaccharides), poly-alpha-hydroxy acid), polyvinyl alcohol (PVA), polyphosphazene, polyoxazolines (POZ), poly(N-acryloylmorpholine), or a combination thereof.

In some embodiments, the hIL10 variant monomer or hIL10 mutein comprising such variant monomer can be conjugated to one or more polyethylene glycol molecules or “PEGylated.” Although the method or site of PEG attachment to the binding molecule may vary, in certain embodiments the PEGylation does not alter, or only minimally alters, the activity of the binding molecule.

PEGs suitable for conjugation to a polypeptide sequence are generally soluble in water at room temperature, and have the general formula

R(O—CH₂—CH₂)ₙO—R,

where R is hydrogen or a protective group such as an alkyl or an alkanol group, and where n is an integer from 1 to 1000. When R is a protective group, it generally has from 1 to 8 carbons. The PEG can be linear or branched. Branched PEG derivatives, “star-PEGs” and multi-armed PEGs are contemplated by the present disclosure.

In some embodiments, selective PEGylation of the hIL10 variant monomer, for example, by the incorporation of non-natural amino acids having side chains to facilitate selective PEG conjugation, may be employed. Specific PEGylation sites can be chosen such that PEGylation of the binding molecule does not affect its binding to the target receptors.

In certain embodiments, the increase in half-life is greater than any decrease in biological activity. PEGs suitable for conjugation to a polypeptide sequence are generally soluble in water at room temperature, and have the general formula R(O—CH2-CH2)nO-R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the binding molecule can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa, or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

In some embodiments, the present disclosure provides a “monoPEGylated” hIL10 mutein (i.e., an hIL10 mutein wherein only one hIL10 monomer of the hIL10 mutein is PEGylated) and a “diPEGylated” hIL10 mutein (i.e., an hIL10 mutein wherein both hIL10 monomers of the hIL10 mutein are PEGylated).

In some instances, the hIL10 variant monomers of the present disclosure possess an N-terminal glutamine (“1Q”) residue. N-terminal glutamine residues have been observed to spontaneously cyclize to form pyroglutamate (pE) at or near physiological conditions. (See e.g., Liu, et al (2011) J. Biol. Chem. 286(13): 11211-11217). In some embodiments, the formation of pyroglutamate prevents N-terminal PEG conjugation particularly when aldehyde chemistry is used for N-terminal PEGylation. This property may be used to provide selective N-terminal PEGylation of one hIL10 variant monomer of an hIL10 mutein comprising such variant monomer. In some embodiments, the hIL10 variant monomers of the present disclosure comprise an amino acid substitution S1Q wherein the naturally occurring N-terminal serine (“S”) residue of hIL10 is replaced with glutamine (“Q”).

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein.

The PEG can be bound to a binding molecule of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of the binding molecules is facilitated by the incorporation of non-natural amino acids bearing unique side chains to facilitate site specific PEGylation. The incorporation of non-natural amino acids into polypeptides to provide functional moieties to achieve site specific PEGylation of such polypeptides is known in the art. See e.g., Ptacin et al., PCT International Application No. PCT/US2018/045257 filed Aug. 3, 2018 and published Feb. 7, 2019 as International Publication Number WO 2019/028419A1.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present disclosure include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g., Sunbright® ME-200AL, NOF), a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10 kDA linear PEG molecules (e.g., Sunbright® GL2-200AL3, NOF), a 20 kDa 2-arm branched PEG-NHS ester the 20 kDA PEG-NHS ester comprising two 10 kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20 kDA linear PEG molecules (e.g., Sunbright® GL2-400AL3), a 40 kDa 2-arm branched PEG-NHS ester the 40 kDA PEG-NHS ester comprising two 20 kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

In some embodiments, a linker can used to join the hIL10 variant monomer and the PEG molecule. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers are described in Section IV. Further, a multimer (e.g., 1, 2, 3, 4, 5, 6, 7, 8, 9, 10, 10-20, 20-30, or 30-50) of these linker sequences may be linked together to provide flexible linkers that may be used to conjugate two molecules. Alternative to a polypeptide linker, the linker can be a chemical linker, e.g., a PEG-aldehyde linker. In some embodiments, the binding molecule is acetylated at the N-terminus by enzymatic reaction with N-terminal acetyltransferase and, for example, acetyl CoA. Alternatively, or in addition to N-terminal acetylation, the binding molecule can be acetylated at one or more lysine residues, e.g., by enzymatic reaction with a lysine acetyltransferase. See, for example Choudhary et al. (2009) Science 325 (5942):834-840.

In some embodiments, the present disclosure provides a hIL10 variant monomer that is PEGylated, wherein the PEG is conjugated to the hIL10 variant monomer and the PEG is a linear or branched PEG molecule having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

### Fatty Acid Carriers

In some embodiments a hIL10 mutein having an extended duration of action in a mammalian subject and useful in the practice of the present disclosure is achieved by covalent attachment of the hIL10 mutein to a fatty acid molecule as described in Resh (2016) Progress in Lipid Research 63: 120-131. Examples of fatty acids that may be conjugated include myristate, palmitate and palmitoleic acid. Myristoylate is typically linked to an N-terminal glycine but lysines may also be myristoylated. Palmitoylation is typically achieved by enzymatic modification of free cysteine —SH groups such as DHHC proteins catalyze S-palmitoylation. Palmitoleylation of serine and threonine residues is typically achieved enzymatically using PORCN enzymes. In some embodiments, the hIL10 mutein is acetylated at the N-terminus by enzymatic reaction with N-terminal acetyltransferase and, for example, acetyl CoA. Alternatively, or in addition to N-terminal acetylation, the hIL10 mutein is acetylated at one or more lysine residues, e.g., by enzymatic reaction with a lysine acetyltransferase. See, for example Choudhary et al. (2009) Science 325 (5942):834L2 ortho840.

### Recombinant Production

In some embodiments, the IL10 molecules of the present disclosure are produced by recombinant DNA technology. In the typical practice of recombinant production of polypeptides, a nucleic acid sequence encoding the desired polypeptide is incorporated into an expression vector suitable for the host cell in which expression will be accomplished, the nucleic acid sequence being operably linked to one or more expression control sequences encoded by the vector and functional in the target host cell. The recombinant protein may be recovered through disruption of the host cell or from the cell medium if a secretion leader sequence (signal peptide) is incorporated into the polypeptide.

In certain embodiments, the hIL10 muteins of the present disclosure contain amino acid substitutions which provide enhanced recombinant expression relative to the expression of wild-type hIL10 or a mutein not containing such substitution; pharmaceutical compositions comprising an hIL10 mutein; recombinant nucleic acid molecules comprising a nucleic acid sequence encoding an hIL10 mutein; recombinant cells engineered to express the hIL10 mutein; and kits comprising the hIL10 mutein, nucleic acids encoding the hIL10 mutein or recombinant cells expressing the hIL10 mutein. In some embodiments, the hIL10 muteins described herein provide substantial increases in yield when expressed in cells while maintaining significant hIL10 biological activity.

In some embodiments, the hIL10 mutein is expressed at higher levels in a transfected or recombinant cell compared to a wild-type or parental IL10 polypeptide. In some embodiments, the hIL10 mutein having increased expression in a transfected or recombinant cell comprises an amino acid substitution at the position corresponding to residue H14 of SEQ ID NO:1. In some embodiments, the amino acid substitution at the position corresponding to H14 of SEQ ID NO:1 is selected from the group consisting of H14A, H14D, H14E, H14I, H14K, H14L, H14M, H14N, H14Q, H14R, H14S, H14T, H14Y, and H14V. In some embodiments, the amino acid substitution at the position corresponding to H14 of SEQ ID NO:1 is selected from H14D, H14C, H14G, H14P, H14F, and H14W. In some embodiments, the amino acid substitutions at the position corresponding to H14 of SEQ ID NO:1 result in substantial increases in yield yet retain STAT3 signaling.

In some embodiments, the IL10 mutein has increased expression in a transfected or recombinant mammalian or bacterial cell compared to a wild-type or parental IL10 polypeptide, and comprises one or more amino acid substitutions at a position corresponding to residues N21, M22, R24, E96, and T100 of SEQ ID NO:1. In some embodiments, the hIL10 mutein has increased expression in a transfected or recombinant cell compared to a wild-type or parental IL10 polypeptide, comprises an amino acid sequence having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1), and comprises one or more amino acid substitutions at a position corresponding to residues N21, M22, R24, E96, and T100 of SEQ ID NO:1. In some embodiments, the one or more amino acid substitutions at a position corresponding to residues N21, M22, R24, E96, and T100 of SEQ ID NO:1 comprise i) a D, E, or K substitution at the position corresponding to N21, ii) a D, S, T, W, or A substitution at the position corresponding to M22, iii) an E substitution at the position corresponding to R24, iv) a Q or K substitution at the position corresponding to E96, and v) a C or L substitution at the position corresponding to T100. In some embodiments, the one or more amino acid substitutions at a position corresponding to N21, M22, R24, E96, and T100 of SEQ ID NO:1 are selected from N21D, N21E, N21K, M22D, M22S, M22T, M22W, M22A, R24E, E96Q, E96K, T100C, T100E, and T100L.

In some embodiments, the IL10 mutein has increased expression in a transfected or recombinant mammalian or bacterial cell compared to a wild-type or parental IL10 polypeptide, and comprises amino acid substitutions corresponding to H14D and N21K, H14D and N21W, H14D and M22S, H14D and M22W, or H14D and T100L with reference to SEQ ID NO:1.

### Combined Mutations:

In some embodiments, the IL10 mutein described herein comprises amino acid substitutions that retain activation of STAT3-mediated signaling in myeloid cells and have increased expression in a transfected or recombinant cell compared to a wild-type or parental IL10 polypeptide. In some embodiments, the hIL10 mutein comprises one or more amino acid substitutions at a position corresponding to residues N18, N21, M22, R32, E74, S93, E96, and T100 of SEQ ID NO:1 combined with an amino acid substitution at a position corresponding to residue H14 of SEQ ID NO:1. In some embodiments, one or more amino acid substitutions at a position corresponding to residues N18, N21, M22, R32, E74, S93, E96, and T100 of SEQ ID NO:1 selected from the group consisting of N18R, N18K, N21C, N21D, N21E, M22D, M22S, M22T, M22W, R32N, R32Q, R32G, R32C, R32P, R32F, R32Y, E74K, S93G, E96C, E96F, E96Y, E96W and T100C are combined with amino acid substitutions at the position corresponding to residue H14 of SEQ ID NO:1 selected from H14C, H14G, H14P, H14F, and H14W.

In some embodiments, one or more amino acid substitutions at a position corresponding to residues N18, N21, M22, R32, E74, S93, E96, and T100 of SEQ ID NO:1 selected from the group consisting of N18R, N18K, N21C, N21D, N21E, M22D, M22S, M22T, M22W, R32N, R32Q, R32G, R32C, R32P, R32F, R32Y, E74K, S93G, E74K, E96C, E96F, E96Y, E96W and T100C are combined with amino acid substitutions at the position corresponding to residue H14 of SEQ ID NO:1 selected from the group consisting of H14A, H14D, H14E, H14I, H14K, H14L, H14M, H14N, H14Q, H14R, H14S, H14T, H14Y, and H14V.

In some embodiments, the hIL10 monomer variant is a polypeptide having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1), and comprises the following amino acid sequence:

SPGQGTQSENSCT (AA14) FPG (AA18) LP (AA21) (AA22) LRDLRDAFS (AA32)
VKTFFQMKDQLDNLLLKESLLEDFKGYLGCQALSEMIQFYLEEVMPQAENQDP
DIKAHVN (AA93) LG (AA96) NLK (AA100) LRLRLRRCHRFLPCENKSKAVEQV
KNAFNKLQEKGIYKAMSEFDIFINYIEAYMTMKIRN

wherein:
AA14 is H (wild-type) or C, G, P, F or W;
AA18 is N (wild-type) or R or K;
AA21 is N (wild-type) or C, D, or E;
AA22 is M (wild-type) or D, S, T, or W;
AA32 is R (wild-type) or N, Q, G, C, P, F, or Y;
AA93 is S (wild-type) or G;
AA96 is E (wild-type) or C, F, Y, or W; and
AA100 is T (wild-type) or C.

In some embodiments, the hIL10 monomer variant is a polypeptide having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1), and comprises the following amino acid sequence:

SPGQGTQSENSCT (AA14) FPG (AA18) LP (AA21) (AA22) LRDLRDAFS (AA32)
VKTFFQMKDQLDNLLLKESLLEDFKGYLGCQALSEMIQFYLEEVMPQAENQDP
DIKA (AA90) V(AA92) (AA93) LG (AA96) NLK (AA100) LRL (AA104) LRRC
HRFLPCENKSKAVEQVKNAFNKLQEKGIYKAMSEFDIFINYIEAYMTMKIRN

wherein:
AA14 is H (wild-type), C, G, P, F, W A, D, E, I, K, L, M, N, Q, R, S, T, Y, or V;
AA18 is N (wild-type), R, K, Y, F, A, D, E, L, V, S, T, I, V, M, or H.
AA21 is N (wild-type), C, D, E, A, R, Q, H, K, S, V, I, L, M, or T;
AA22 is M (wild-type), D, S, T, W, A, V, I, L, N, or Q;
AA32 is R (wild-type), N, Q, G, C, P, F, Y, A, D, E, L, V, S, T, I, V, M, or H;
AA90 is H (wild-type), A, D, E, I, K, L, M, N, Q, R, S, T, Y, or V
AA92 is N (wild-type), D, Q, E, H, K, S, V, I, L, M, T, or A;
AA93 is S (wild-type), G, E, A, R, N, D, Q, E, I, L, K, M, or V;
AA96 is E (wild-type), C, F, Y, W A, N, D, Q, H, K, or S;
AA100 is T (wild-type), C, D, V, E, A, R, N, Q, E, I, L, K, M, or S;
AA104 is R (wild type), A, W, Y, F, H, D, E, N, Q, S, T, I, L, V, or M.

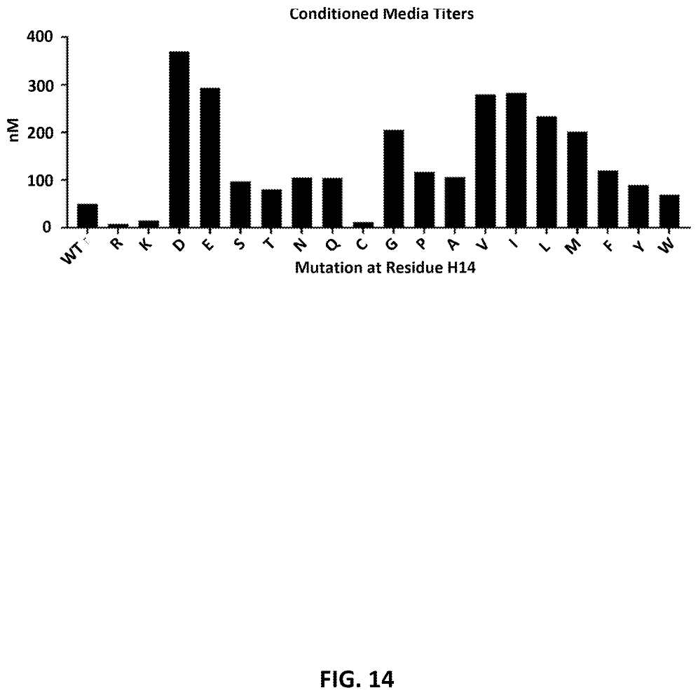

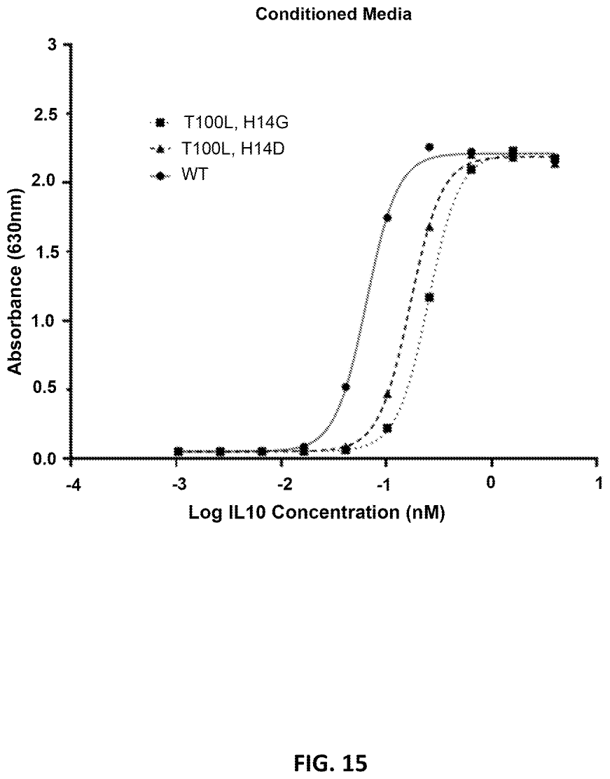

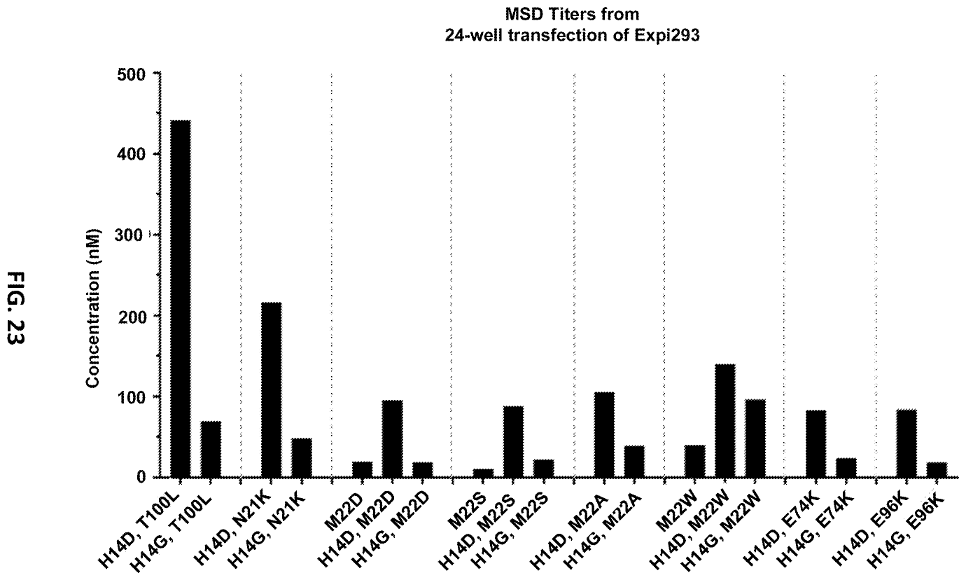

hIL10 muteins comprising hIL10 variant monomers having a mutation at a position corresponding to amino acid residue H14 of SEQ ID NO:1 were evaluated for expression in the HEK293 cell line. The results are shown in FIG. 14. Based on the data, hIL10 variant monomers comprising amino acid substitutions H14D or H14G were further evaluated in combination with amino acid substitutions at positions corresponding to residues N21, M22, E74, E96, and T100 of SEQ ID NO:1. As shown in FIGS. 15 to 22, H14G or H14D mutations combined with T100L, N21K, M22S, M22A, M22W, and E74K demonstrated hIL10 activity in the STAT3 reporter assay. FIG. 23 shows the level of expression of the amino acid substitutions at position H14 combined with amino acid substitutions at positions N21, M22, E74, E96, and T100. The data demonstrates that amino acid substitutions at position H14 can increase the relative expression of hIL10 variant monomers in a mammalian cell line.

Nucleic Acid Sequences Encoding hIL10 Muteins:

In some embodiments, the the hIL10 mutein or hIL10 monomer variant is produced by recombinant methods using a nucleic acid sequence encoding the hIL10 mutein or hIL10 monomer variant (or fusion protein comprising the hIL10 mutein or hIL10 monomer variant). The nucleic acid sequence encoding the desired the hIL10 mutein or hIL10 monomer variant can be synthesized by chemical means using an oligonucleotide synthesizer.

In some embodiments, the hIL10 mutein or hIL10 monomer variant is produced by recombinant methods using a nucleic acid sequence encoding the hIL10 mutein (or fusion protein comprising the hIL10 mutein). The nucleic acid sequence encoding the desired h the hIL10 mutein or hIL10 monomer variant can be synthesized by chemical means using an oligonucleotide synthesizer.

The nucleic acid molecules are not limited to sequences that encode polypeptides; some or all of the non-coding sequences that lie upstream or downstream from a coding sequence can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The nucleic acid molecules encoding the the hIL10 mutein or hIL10 monomer variant (and fusions thereof) may contain naturally occurring sequences or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

Nucleic acid sequences encoding the hIL10 mutein may be obtained from various commercial sources that provide custom made nucleic acid sequences. Amino acid sequence variants of the hIL10 mutein of the present disclosure are prepared by introducing appropriate nucleotide changes into the coding sequence based on the genetic code which is well known in the art. Such variants represent insertions, substitutions, and/or specified deletions of, residues as noted. Any combination of insertion, substitution, and/or specified deletion is made to arrive at the final construct, provided that the final construct possesses the desired biological activity as defined herein.

Methods for constructing a DNA sequence encoding a the hIL10 mutein or hIL10 monomer variant and expressing those sequences in a suitably transformed host include, but are not limited to, using a PCR-assisted mutagenesis technique. Mutations that consist of deletions or additions of amino acid residues to a hIL10 mutein can also be made with standard recombinant techniques. In the event of a deletion or addition, the nucleic acid molecule encoding a hIL10 mutein is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

A hIL10 mutein or hIL10 monomer variant of the present disclosure may be produced recombinantly not only directly, but also as a fusion polypeptide with a heterologous polypeptide, e.g. a signal sequence or other polypeptide having a specific cleavage site at the N-terminus or C-terminus of the mature hIL10 mutein. In some embodiments, the nucleic acid molecule further comprises a nucleic acid sequence encoding a signal peptide In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. The inclusion of a signal sequence depends on whether it is desired to secrete the hIL10 mutein from the recombinant cells in which it is made. If the chosen cells are prokaryotic, it generally is preferred that the DNA sequence not encode a signal sequence. When the recombinant host cell is a yeast cell such as Saccharomyces cerevisiae, the alpha mating factor secretion signal sequence may be employed to achieve extracellular secretion of the hIL10 mutein into the culture medium as described in Singh, U.S. Pat. No. 7,198,919 B1 issued Apr. 3, 2007. In some embodiments, the signal peptide comprises an endogenous or wild-type IL10 signal peptide. In some embodiments, the signal peptide comprises the amino acid sequence of the human IL10 polypeptide: MHSSALLCCLVLLTGVRA (SEQ ID NO:86). In some embodiments, the signal peptide comprises the amino acid sequence of murine IL10 polypeptide: MPGSALLCCLLLLTGMRI (SEQ ID NO:87).

In the event the hIL10 mutein or hIL10 monomer variant to be expressed is to be expressed as a chimera (e.g., a fusion protein comprising a hIL10 mutein or hIL10 monomer variant and a heterologous polypeptide sequence), the chimeric protein can be encoded by a hybrid nucleic acid molecule comprising a first sequence that encodes all or part of hIL10 mutein or hIL10 monomer variant and a second sequence that encodes all or part of the heterologous polypeptide. For example, subject hIL10 mutein or hIL10 monomer variant described herein may be fused to a hexa-/octa-histidine tag to facilitate purification of bacterially expressed protein, or to a hemagglutinin tag to facilitate purification of protein expressed in eukaryotic cells. By first and second, it should not be understood as limiting to the orientation of the elements of the fusion protein and a heterologous polypeptide can be linked at either the N-terminus and/or C-terminus of the hIL10 mutein. For example, the N-terminus may be linked to a targeting domain and the C-terminus linked to a hexa-histidine tag purification handle.

The complete amino acid sequence of the polypeptide (or fusion/chimera) to be expressed can be used to construct a back-translated gene. A DNA oligomer containing a nucleotide sequence coding a hIL10 mutein can be synthesized. For example, several small oligonucleotides coding for portions of the desired polypeptide can be synthesized and then ligated. The individual oligonucleotides typically contain 5′ or 3′ overhangs for complementary assembly.

### Codon Optimization:

In some embodiments, the nucleic acid sequence encoding the hIL10 mutein or hIL10 monomer variant may be “codon optimized” to facilitate expression in a particular host cell type. Techniques for codon optimization in a wide variety of expression systems, including mammalian, yeast and bacterial host cells, are well known in the and there are online tools to provide for a codon optimized sequences for expression in a variety of host cell types. See e.g. Hawash, et al., (2017) 9:46-53 and Mauro and Chappell in Recombinant Protein Expression in Mammalian Cells: Methods and Protocols, edited by David Hacker (Human Press New York). Additionally, there are a variety of web based on-line software packages that are freely available to assist in the preparation of codon optimized nucleic acid sequences.

### Expression Vectors:

Once assembled (by synthesis, site-directed mutagenesis or another method), the nucleic acid sequence encoding an a hIL10 mutein will be inserted into an expression vector. A variety of expression vectors for uses in various host cells are available and are typically selected based on the host cell for expression. An expression vector typically includes, but is not limited to, one or more of the following: an origin of replication, one or more marker genes, an enhancer element, a promoter, and a transcription termination sequence. Vectors include viral vectors, plasmid vectors, integrating vectors, and the like. Plasmids are examples of non-viral vectors.

In some embodiments, such as where the first and second hIL10 monomers of the hIL10 mutein are different such as in the context of heterodimeric hIL10 muteins, the vector comprises a first nucleic acid sequence encoding the first hIL10 monomer and as second nucleic acid sequence encoding the first hIL10 monomers where the first and second nucleic acid sequences are operably linked to an expression control element (e.g. a promoter) and the first and second nucleic acid sequences are separated by a sequence which facilitates co-expression (e.g. an IRES or T2A sequence). Alternatively, the vector comprises a first nucleic acid sequence encoding the first hIL10 monomer and a second nucleic acid sequence encoding the first hIL10 monomer where the first and second nucleic acid sequences are each operably linked to an expression control sequence, the expression control sequences being the same or different.

To facilitate efficient expression of the recombinant polypeptide, the nucleic acid sequence encoding the polypeptide sequence to be expressed is operably linked to transcriptional and translational regulatory control sequences that are functional in the chosen expression host.

### Selectable Marker:

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

### Regulatory Control Sequences:

Expression vectors for a hIL10 mutein or hIL10 monomer variant of the present disclosure contain a regulatory sequence that is recognized by the host organism and is operably linked to a nucleic acid sequence encoding the hIL10 mutein. The terms “regulatory control sequence,” “regulatory sequence” or “expression control sequence” are used interchangeably herein to refer to promoters, enhancers, and other expression control elements (e.g., polyadenylation signals). See, for example, Goeddel (1990) in Gene Expression Technology: Methods in Enzymology 185 (Academic Press, San Diego CA USA Regulatory sequences include those that direct constitute expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. In selecting an expression control sequence, a variety of factors understood by one of skill in the art are to be considered. These include, for example, the relative strength of the sequence, its controllability, and its compatibility with the actual DNA sequence encoding the subject a hIL10 mutein, particularly as regards potential secondary structures.

### Promoters:

In some embodiments, the regulatory sequence is a promoter, which is selected based on, for example, the cell type in which expression is sought. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

Transcription from vectors in mammalian host cells may be controlled, for example, by promoters obtained from the genomes of viruses such as polyoma virus, fowlpox virus, adenovirus (such as human adenovirus serotype 5), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

### Enhancers:

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter. Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Additional examples of marker or reporter genes include beta-lactamase, chloramphenicol acetyltransferase (CAT), adenosine deaminase (ADA), dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). Those of skill in the art can readily determine whether a given regulatory element or selectable marker is suitable for use in a particular experimental context.

Proper assembly of the expression vector can be confirmed by nucleotide sequencing, restriction mapping, and expression of a biologically active polypeptide in a suitable host.

### Host Cells:

The present disclosure further provides prokaryotic or eukaryotic cells that contain and express one or more nucleic acid molecules that encoding a hIL10 variant monomer or hIL10 mutein. A cell of the present disclosure is a transfected cell, i.e., a cell into which a nucleic acid molecule, for example a nucleic acid molecule encoding a hIL10 variant monomer, has been introduced by means of recombinant DNA techniques.

In some embodiments, such as where the first and second hIL10 monomers of the hIL10 mutein are different such as in the context of heterodimeric hIL10 muteins, the recombinantly modified cell comprises a vector, the vector comprising a first nucleic acid sequence encoding a first hIL10 monomer and a second nucleic acid sequence encoding the second hIL10 monomer where the first and second nucleic acid sequences are operably linked to a single expression control sequence and the first and second nucleic acid sequences are separated by a sequence which facilitates co-expression. In other embodiments, the recombinantly modified cell comprises a vector, the vector comprising a first nucleic acid sequence encoding a first hIL10 monomer and a second nucleic acid sequence encoding the second hIL10 monomer where the first and second nucleic acid sequences are each operably linked to a expression control sequence. In other embodiments, where the first and second hIL10 monomers of the hIL10 mutein are different such as in the context of heterodimeric hIL10 muteins, the recombinantly modified cell may comprise two vectors, first, the vector comprising a first nucleic acid sequence encoding a first hIL10 monomer operably linked to an expression control sequence and a second vector comprising a nucleic acid sequence encoding the second hIL10 monomer In some embodiments, the recombinantly modified cell is a prokaryotic cell, such as a bacterial cell. In some embodiments, the recombinantly modified cell is a eukaryotic cell, such as a mammalian cell.

Host cells are typically selected in accordance with their compatibility with the chosen expression vector, the toxicity of the product coded for by the DNA sequences of this invention, their secretion characteristics, their ability to fold the polypeptides correctly, their fermentation or culture requirements, and the ease of purification of the products coded for by the DNA sequences. Suitable host cells for cloning or expressing the DNA in the vectors herein are the prokaryote, yeast, or higher eukaryote cells.

In some embodiments the recombinant hIL10 mutein or hIL10 variant monomer can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerenvisiae include pYepSecl (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)).

Examples of useful mammalian host cell lines are mouse L cells (L-M[TK-], ATCC #CRL-2648), monkey kidney CV1 line transformed by SV40 (COS-7, ATCC CRL 1651); human embryonic kidney line (HEK293 or HEK293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2). In mammalian cells, the expression vector's control functions are often provided by viral regulatory elements. For example, commonly used promoters are derived from polyoma, Adenovirus 2, cytomegalovirus, and Simian Virus 40.

The hIL10 mutein or hIL10 variant monomer may be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

In some embodiments, a hIL10 mutein or hIL10 variant monomer obtained will be glycosylated or unglycosylated depending on the host organism used to produce the mutein. If bacteria are chosen as the host then the hIL10 mutein produced will be unglycosylated. Eukaryotic cells, on the other hand, will typically result in glycosylation of the hIL10 mutein.

In some embodiments, it is possible that an amino acid sequence (particularly a CDR sequence) of an sdAb to be incorporated into a hIL10 mutein may contain a glycosylation motif, particularly an N-linked glycosylation motif of the sequence Asn-X-Ser (N-X-S) or Asn-X-Thr (N-X-T), wherein X is any amino acid except for proline. In such instances, it is desirable to eliminate such N-linked glycosylation motifs by modifying the sequence of the N-linked glycosylation motif to prevent glycosylation. In some embodiments, the N-linked glycosylation motif is disrupted by the incorporation of conservative amino acid substitution of the Asn (N) residue of the N-linked glycosylation motif.

For other additional expression systems for both prokaryotic and eukaryotic cells, see Chapters 16 and 17 of Sambrook et al. (1989) Molecular Cloning: A Laboratory Manual (2nd ed., Cold Spring Harbor Laboratory Press, Plainview, N.Y.). See, Goeddel (1990) in Gene Expression Technology: Methods in Enzymology 185 (Academic Press, San Diego, Calif.).

### Transfection:

The expression constructs of the can be introduced into host cells to thereby produce a hIL10 mutein or hIL10 variant monomer disclosed herein. The expression vector comprising a nucleic acid sequence encoding hIL10 mutein is introduced into the prokaryotic or eukaryotic host cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals. To facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

### Cell Culture:

Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

### Recovery of Recombinant Proteins:

Recombinantly produced hIL10 muteins or hIL10 variant monomers can be recovered from the culture medium as a secreted polypeptide if a secretion leader sequence is employed. Alternatively, the hIL10 muteins or hIL10 variant monomers can also be recovered from host cell lysates. A protease inhibitor, such as phenyl methyl sulfonyl fluoride (PMSF) may be employed during the recovery phase from cell lysates to inhibit proteolytic degradation during purification, and antibiotics may be included to prevent the growth of adventitious contaminants.

Various purification steps are known in the art and find use, e.g. affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural specific binding of one molecular species to separate and purify a second species from a mixture. Antibodies are commonly used in affinity chromatography. Size selection steps may also be used, e.g. gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column.

A recombinantly hIL10 mutein or hIL10 variant monomer expressed by the transformed host can be purified according to any suitable method. Recombinant hIL10 muteins or hIL10 variant monomers can be isolated from inclusion bodies generated in E. coli, or from conditioned medium from either mammalian or yeast cultures producing a given mutein using cation exchange, gel filtration, and or reverse phase liquid chromatography. The substantially purified forms of the recombinant a hIL10 mutein or hIL10 variant monomer can be purified from the expression system using routine biochemical procedures, and can be used, e.g., as therapeutic agents, as described herein.

In some embodiments, where the hIL10 mutein or hIL10 variant monomer is expressed with a purification tag as discussed above, this purification handle may be used for isolation of the hIL10 mutein or hIL10 variant monomer from the cell lysate or cell medium. Where the purification tag is a chelating peptide, methods for the isolation of such molecules using immobilized metal affinity chromatography are well known in the art. See, e.g., Smith, et al. U.S. Pat. No. 4,569,794.

The biological activity of the hIL10 mutein or hIL10 variant monomer recovered can be assayed for activating by any suitable method known in the art and may be evaluated as substantially purified forms or as part of the cell lysate or cell medium when secretion leader sequences are employed for expression.

### Pharmaceutical Formulations

In some embodiments, the subject hIL10 mutein (and/or nucleic acids encoding the hIL10 mutein or recombinant cells incorporating a nucleic acid sequence and modified to express the hIL10 mutein) can be incorporated into compositions, including pharmaceutical compositions. Such compositions typically include the polypeptide or nucleic acid molecule and a pharmaceutically acceptable carrier. A pharmaceutical composition is formulated to be compatible with its intended route of administration and is compatible with the therapeutic use for which the hIL10 mutein is to be administered to the subject in need of treatment or prophyaxis.

In some embodiments, the present disclosure provides a pharmaceutical composition comprising a “monoPEGylated” hIL10 mutein and a “diPEGylated” hIL10 mutein. In some embodiments, the ratio of the monoPEGylated hIL10 mutein and the diPEGylated hIL10 mutein species in such composition are approximately 1:1. In some embodiments, the present disclosure provides a hIL10 mutein composition comprising a mixture of a nonPEGylated hIL10 mutein, a monopegylated hIL10 mutein and a diPEGylated hIL10 mutein and one or more pharmaceutically acceptable carriers.

### Carriers:

Carriers include a sterile diluent such as water for injection, saline solution, fixed oils, polyethylene glycols, glycerine, propylene glycol or other synthetic solvents. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. For intravenous administration, suitable carriers include physiological saline, bacteriostatic water, Cremophor ELTM (BASF, Parsippany, N.J.) or phosphate buffered saline (PBS).

### Buffers:

The term buffers includes buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5).

### Dispersions:

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### Preservatives:

The pharmaceutical formulations for parenteral administration to a subject should be sterile and should be fluid to facilitate easy syringability. It should be stable under the conditions of manufacture and storage and are preserved against the contamination. Prevention of the action of microorganisms can be achieved by various antibacterial and antifungal agents, for example, agents such as benzyl alcohol or methyl parabens; antioxidants such as ascorbic acid or sodium bisulfite; chelating agents such as ethylenediaminetetraacetic acid, parabens, chlorobutanol, phenol, ascorbic acid, thimerosal, and the like. Sterile solutions can be prepared by incorporating the active compound in the required amount in an appropriate solvent with one or a combination of ingredients enumerated above, as required, followed by filtered sterilization.

### Tonicity Agents:

In many cases, it will be preferable to include isotonic agents, for example, sugars, polyalcohols such as mannitol, sorbitol, sodium chloride in the composition.

### Routes of Administration

In some embodiments of the therapeutic methods of the present disclosure involve the administration of a pharmaceutical formulation comprising a hIL10 mutein (and/or nucleic acids encoding the hIL10 mutein or recombinantly modified host cells expressing the hIL10 mutein) to a subject in need of treatment. The pharmaceutical formulation comprising a hIL10 mutein of the present disclosure may be administered to a subject in need of treatment or prophyaxis by a variety of routes of administration, including parenteral administration, oral, topical, or inhalation routes.

### Parenteral Administration:

In some embodiments, the methods of the present disclosure involve the parenteral administration of a pharmaceutical formulation comprising a hIL10 mutein (and/or nucleic acids encoding the hIL10 mutein or recombinantly modified host cells expressing the hIL10 mutein) to a subject in need of treatment. Examples of parenteral routes of administration include, for example, intravenous, intradermal, subcutaneous, transdermal (topical), transmucosal, and rectal administration. Parenteral formulations comprise solutions or suspensions used for parenteral application can include vehicles the carriers and buffers. Pharmaceutical formulations for parenteral administration include sterile aqueous solutions (where water soluble) or dispersions and sterile powders for the extemporaneous preparation of sterile injectable solutions or dispersion. The parenteral preparation can be enclosed in ampoules, disposable syringes or multiple dose vials made of glass or plastic. In one embodiment, the formulation is provided in a prefilled syringe for

### Oral Administration:

In some embodiments, the methods of the present disclosure involve the oral administration of a pharmaceutical formulation comprising a hIL10 mutein (and/or nucleic acids encoding the hIL10 mutein or recombinantly modified host cells expressing the hIL10 mutein) to a subject in need of treatment. Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

### Inhalation Formulations:

In some embodiments, the methods of the present disclosure involve the inhaled administration of a pharmaceutical formulation comprising a hIL10 mutein (and/or nucleic acids encoding the hIL10 mutein or recombinantly modified host cells expressing the hIL10 mutein) to a subject in need of treatment. In the event of administration by inhalation, subject hIL10 muteins, or the nucleic acids encoding them, are delivered in the form of an aerosol spray from pressured container or dispenser which contains a suitable propellant, e.g., a gas such as carbon dioxide, or a nebulizer. Such methods include those described in U.S. Pat. No. 6,468,798.

### Mucosal and Transdermal Formulations:

In some embodiments, the methods of the present disclosure involve the mucosal or transdermal administration of a pharmaceutical formulation comprising a hIL10 mutein (and/or nucleic acids encoding the hIL10 mutein or recombinantly modified host cells expressing the hIL10 mutein) to a subject in need of treatment. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories suppositories (e.g., with conventional suppository bases such as cocoa butter and other glycerides) or retention enemas for rectal delivery. For transdermal administration, the active compounds are formulated into ointments, salves, gels, or creams as generally known in the art and may incorporate permeation enhancers such as ethanol or lanolin.

### Extended Release and Depot Formulations:

In some embodiments of the method of the present disclosure, the hIL10 mutein is administered to a subject in need of treatment in a formulation to provide extended release of the hIL10 mutein agent. Examples of extended release formulations of the injectable compositions can be brought about by including in the composition an agent which delays absorption, for example, aluminum monostearate and gelatin. In one embodiment, the subject hIL10 mutein or nucleic acids are prepared with carriers that will protect the hIL10 mutein against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Administration of Nucleic Acids Encoding the hIL10 Mutein:

In some embodiments of the method of the present disclosure, delivery of the hIL10 mutein to a subject in need of treatment is achieved by the administration of a nucleic acid encoding the hIL10 mutein. Methods for the administration nucleic acid encoding the hIL10 mutein to a subject is achieved by transfection or infection using methods known in the art, including but not limited to the methods described in McCaffrey et al. (Nature (2002) 418:6893), Xia et al. (Nature Biotechnol. (2002) 20:1006-1010), or Putnam (Am. J. Health Syst. Pharm. (1996) 53: 151-160 erratum at Am. J. Health Syst. Pharm. (1996) 53:325). In some embodiments, the hIL10 mutein is administered to a subject by the administration of a pharmaceutically acceptable formulation of recombinant expression vector comprising a nucleic acid sequence encoding the hIL10 mutein operably linked to one or more expression control sequences operable in a mammalian subject. In some embodiments, the expression control sequence may be selected that is operable in a limited range of cell types (or single cell type) to facilitate the selective expression of the hIL10 mutein in a particular target cell type. In one embodiment, the recombinant expression vector is a viral vector. In some embodiments, the recombinant vector is a recombinant viral vector. In some embodiments the recombinant viral vector is a recombinant adenoassociated virus (rAAV) or recombinant adenovirus (rAd), in particular a replication deficient adenovirus derived from human adenovirus serotypes 3 and/or 5. In some embodiments, the replication deficient adenovirus has one or more modifications to the E1 region which interfere with the ability of the virus to initiate the cell cycle and/or apoptotic pathways in a human cell. The replication deficient adenoviral vector may optionally comprise deletions in the E3 domain. In some embodiments the adenovirus is a replication competent adenovirus. In some embodiments the adenovirus is a replication competent recombinant virus engineered to selectively replicate in the target cell type.

In some embodiments, particularly for administration of hIL10 muteins to the subject, particular for treatment of diseases of the intestinal tract or bacterial infections in a subject, the nucleic acid encoding the hIL10 mutein may be delivered to the subject by the administration of a recombinantly modified bacteriophage vector encoding the hIL10 mutein. As used herein, the terms “procaryotic virus,” “bacteriophage” and “phage” are used interchangeably hereinto describe any of a variety of bacterial viruses that infect and replicate within a bacterium. Bacteriophage selectively infect procaryotic cells, restricting the expression of the hIL10 mutein to procaryotic cells in the subject while avoiding expression in mammalian cells. A wide variety of bacteriophages capable of selection a broad range of bacterial cells have been identified and characterized extensively in the scientific literature. In some embodiments, the phage is modified to remove adjacent motifs (PAM). Elimination of Cas9 sequences from the phage genome reduces ability of the Cas9 endonuclease of the target procaryotic cell to neutralize the invading phage encoding the hIL10 mutein.

Administration of Recombinantly Modified Cells Expressing the hIL10 Mutein

In some embodiments of the method of the present disclosure, delivery of the hIL10 mutein to a subject in need of treatment is achieved by the administration of recombinant host cells modified to express the hIL10 mutein may be administered in the therapeutic and prophylactic applications described herein. In some embodiments, the recombinant host cells are mammalian cells, e.g., human cells.

In some embodiments, the nucleic acid sequence encoding the hIL10 mutein (or vectors comprising same) may be maintained extrachromosomally in the recombinantly modified host cell for administration. In other embodiments, the nucleic acid sequence encoding the hIL10 mutein may be incorporated into the genome of the host cell to be administered using at least one endonuclease to facilitate incorporate insertion of a nucleic acid sequence into the genomic sequence of the cell. As used herein, the term “endonuclease” is used to refer to a wild-type or variant enzyme capable of catalyzing the cleavage of bonds between nucleic acids within a DNA or RNA molecule, preferably a DNA molecule. Endonucleases are referred to as “rare-cutting” endonucleases when such endonucleases have a polynucleotide recognition site greater than about 12 base pairs (bp) in length, more preferably of 14-55 bp. Rare-cutting endonucleases can be used for inactivating genes at a locus or to integrate transgenes by homologous recombination (HR) i.e. by inducing DNA double-strand breaks (DSBs) at a locus and insertion of exogenous DNA at this locus by gene repair mechanism. Examples of rare-cutting endonucleases include homing endonucleases (Grizot, et al (2009) Nucleic Acids Research 37(16):5405-5419), chimeric Zinc-Finger nucleases (ZFN) resulting from the fusion of engineered zinc-finger domains (Porteus M and Carroll D., Gene targeting using zinc finger nucleases (2005) Nature Biotechnology 23(3):967-973, a TALEN-nuclease, a Cas9 endonuclease from CRISPR system as or a modified restriction endonuclease to extended sequence specificity (Eisenschmidt, et al. 2005; 33(22): 7039-7047).

In some embodiments, particularly for administration of hIL10 muteins to the intestinal tract, the hIL10 mutein may be delivered to the subject by a recombinantly modified procaryotic cell (e.g., Lactobacillus lacti). The use of engineered procaryotic cells for the delivery of recombinant proteins to the intestinal tract are known in the art. See, e.g. Lin, et al. (2017) Microb Cell Fact 16:148. In some embodiments, the engineered bacterial cell expressing the hIL10 mutein may be administered orally, typically in aqueous suspension, or rectally (e.g. enema).

### Methods of Use

The present disclosure further provides methods of treating a subject suffering from a disease disorder or condition by the administration of a therapeutically effective amount of a hIL10 mutein (or nucleic acid encoding a hIL10 mutein including recombinant viruses encoding the hIL10 mutein) of the present disclosure.

### Inflammatory and Autoimmune Disorders

Disorders amenable to treatment with a hIL10 mutein (including pharmaceutically acceptable formulations comprising a hIL10 mutein and/or the nucleic acid molecules that encode them including recombinant viruses encoding such a hIL10 mutein) of the present disclosure include inflammatory or autoimmune diseases including but not limited to, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes including Type 1 or type 2 diabetes, inflammation, autoimmune disease, atopic diseases, paraneoplastic autoimmune diseases, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome (Seronegativity Enthesopathy Arthropathy Syndrome), juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome(Seronegativity, Enthesopathy, Arthropathy Syndrome).

Other examples of proliferative and/or differentiative disorders amenable to treatment with hIL10 mutein (including pharmaceutically acceptable formulations comprising hIL10 mutein and/or the nucleic acid molecules that encode them including recombinant viruses encoding such hIL10 mutein) of the present disclosure include, but are not limited to, skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the Stratum basale (Stratum germinativum), Stratum spinosum, Stratum granulosum, Stratum lucidum or Stratum corneum. In other embodiments, the disorder may involve aberrant activity of a dermal cell, for example, a dermal endothelial, fibroblast, immune cell (e.g., mast cell or macrophage) found in a dermal layer, for example, the papillary layer or the reticular layer.

Examples of inflammatory or autoimmune skin disorders include psoriasis, psoriatic arthritis, dermatitis (eczema), for example, exfoliative dermatitis or atopic dermatitis, Pityriasis rubra pilaris, pityriasis rosacea, parapsoriasis, pityriasis lichenoiders, Lichen planus, Lichen nitidus, ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; Lichen planus; and keratitis. The skin disorder can be dermatitis, e.g., atopic dermatitis or allergic dermatitis, or psoriasis.

The compositions of the present disclosure (including pharmaceutically acceptable formulations comprising hIL10 mutein and/or the nucleic acid molecules that encode them including recombinant viruses encoding such hIL10 mutein) can also be administered to a patient who is suffering from (or may suffer from) psoriasis or psoriatic disorders. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, plaque psoriasis, moderate to severe plaque psoriasis, psoriasis vulgaris, eruptive psoriasis, psoriatic erythroderma, generalized pustular psoriasis, annular pustular psoriasis, or localized pustular psoriasis.

### Methods for Modulating IL10-Mediated Signaling

In another aspect, the disclosure provides methods for modulating IL10-mediated signaling in a subject. In some embodiments, the method comprises administering to the subject an effective amount of a pharmaceutical composition to the subject, where the pharmaceutical composition comprises a hIL10 mutein described herein, a nucleic acid molecule encoding a hIL10 mutein described herein, a recombinantly modified cell comprising a nucleic acid molecule encoding a hIL10 mutein described herein. In some embodiments, the pharmaceutical composition comprises a pharmaceutically acceptable carrier.

In some embodiments, the method for modulating IL10-mediated signaling in a subject comprises determining STAT3-mediated signaling in one or more cells obtained from the subject. In some embodiments, the STAT3-mediated signaling is determined by an assay selected from the group consisting of a gene expression assay, a phospho-flow signaling assay, and an enzyme-linked immunosorbent assay (ELISA). In some embodiments, the STAT3-mediated signaling in the subject is reduced by about 20% to about 100% compared to a reference level. In some embodiments, the administered composition results in a reduced capacity to induce expression of a pro-inflammatory gene selected from IFN-γ, granzyme B, granzyme A, perforin, TNF-α, GM-CSF, and MIP1α in the subject.

### Kits

Also provided are kits comprising the hIL10 muteins of the disclosure. In some embodiments, the kit comprises one or more components for modulating IL10-mediated signaling in a subject, or treating a health condition in a subject in need thereof, wherein the components are selected from a hIL10 mutein described herein, a nucleic acid molecule encoding a hIL10 mutein described herein, a recombinantly modified cell comprising a nucleic acid molecule encoding a hIL10 mutein described herein, or a pharmaceutical composition comprising one of more the components. In some embodiments, the pharmaceutical composition of the kit comprises a pharmaceutically acceptable carrier.

### EXAMPLES

The following Examples are provided to illustrate, but not to limit the claimed invention.

### Construction of IL10 Muteins

All IL10 muteins were constructed using standard mutagenesis techniques known in the art. The nucleic acid and amino acid sequences for each of the mutants is set forth in Table 1 (including sequences encoding a heterologous signal peptide, a histidine purification tag, and a GS linker). The amino acid sequences for each of the mutants is set forth in Table 2.

### Generation of the Human IL10 Mammalian Expression Vectors

A pCDNA3.4 mammalian expression vector (Life Technologies, Carlsbad, CA) was modified to include additional restriction sites in the Multiple Cloning Cloning Site (MCS) and renamed pExSyn2.0. The human IL-10 Open Reading Frame (ORF) was cloned into pExSyn2.0 at the EcoRI and NotI restriction sites and incorporated an N-terminal “GS” Linker and an 8× Histidine tag using standard molecular biology cloning techniques. The resultant vector was named “pExSyn2.0—His-hIL10 WT”. The vector was DNA sequenced (MC Lab, South San Francisco, CA) to confirm identity.

### Expression of Human IL10 Mutants in Expi293 Expression System

The pExSyn2.0—His-hiL10 WT and mutein vectors expression vectors were transfected into Expi293 Cells following the manufacturer's recommended protocol (Life Technologies, Carlsbad, CA), with the following exceptions: 20% total volume of fresh growth medium and 75% of the recommended Enhancer and feed were added at day 1 post transfection. The cultures were harvested when the viability was ˜60-70%.

HEK-Blue Human IL10 pSTAT3 Reporter Assay

To characterize the mutations' effects on pSTAT3 signaling, the Human IL10 wildtype and muteins were run in the HEK-Blue Human IL10 pSTAT3 Reporter Assay (Invivogen, San Diego, CA). The manufacturer's protocol was followed with the following notes: The proteins were serially diluted in concentrations ranging from 4 nM to 4.85E-4 nM. The results of the pSTAT3 Reporter Assay are shown in FIGS. 2-13, 15-22 and 24-33. All muteins tested showed reduced pSTAT3 activity relative to wild type human IL10 as judged by increased EC50.

### Expression Titering of Human IL10 and Human IL10 Mutants

Protein concentration levels from harvested media for all expressed human IL-10 proteins (wildtype and muteins) were quantified with the U-PLEX Human IL-10 Assay (Meso Scale Diagnostics, Rockville, MD) following the manufacturer's recommended protocol, with the following exception: samples were initially diluted 1:10, then serially diluted 10-fold for a total of 8 dilutions.

TABLE 1
Wildtype hIL-10 and mutant hIL-10 Sequences

| | | | |
|---|---|---|---|
| | | Vector | |
| Description: | Human IL-10, Wildtype | Name: | pExSyn2.0-His-hIL10 WT (mp 43) |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 5 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCAGAGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACTCCCTGGGGGAGAACCTGAAGACCCTCAG | |
| | GCTGAGGCTACGGCGCTGTCATCGATTTCTTCCCTGTGAA | |
| | AACAAGAGCAAGGCCGTGGAGCAGGTGAAGAATGCCTT | |
| | TAATAAGCTCCAAGAGAAAGGCATCTACAAAGCCATGAG | |
| | TGAGTTTGACATCTTCATCAACTACATAGAAGCCTACATG | |
| | ACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSRVKTFFQMKDQLDNLLLKESL | No: 6 |
| | LEDFKGYLGCQALSEMIQFYLEEVMPQAENQDPDIKAHVN | |
| | SLGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, N21C | Name: | 078 pExSyn2.0-huIL10-8X NHis-N21C |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 7 |
| | ACGGATCCAGCCCAGGCCAGGGCACCCAGTCTGAGAACA | |
| | GCTGCACCCACTTCCCAGGCAACCTGCCTTGCATGCTTCG | |
| | AGATCTCCGAGATGCCTTCAGCAGAGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACTCCCTGGGGGAGAACCTGAAGACCCTCAG | |
| | GCTGAGGCTACGGCGCTGTCATCGATTTCTTCCCTGTGAA | |
| | AACAAGAGCAAGGCCGTGGAGCAGGTGAAGAATGCCTT | |
| | TAATAAGCTCCAAGAGAAAGGCATCTACAAAGCCATGAG | |
| | TGAGTTTGACATCTTCATCAACTACATAGAAGCCTACATG | |
| | ACAATGAAGATACGAAACTGATAA | |
| PROTEIN | MGWSCIILFLVATATGVHSAHHHHHHHHGSSPGQGTQSENSC | SEQ ID |
| | THFPGNLPCMLRDLRDAFSRVKTFFQMKDQLDNLLLKESL | No: 8 |
| | LEDFKGYLGCQALSEMIQFYLEEVMPQAENQDPDIKAHVN | |
| | SLGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, N21D | Name: | 073 pExSyn2.0-huIL10-8X NHis-N21D |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 9 |
| | ACGGATCCAGCCCAGGCCAGGGCACCCAGTCTGAGAACA | |
| | GCTGCACCCACTTCCCAGGCAACCTGCCTGACATGCTTC | |
| | GAGATCTCCGAGATGCCTTCAGCAGAGTGAAGACTTTCT | |
| | TTCAAATGAAGGATCAGCTGGACAACTTGTTGTTAAAGG | |
| | AGTCCTTGCTGGAGGACTTTAAGGGTTACCTGGGTTGCC | |
| | AAGCCTTGTCTGAGATGATCCAGTTTTACCTGGAGGAGG | |
| | TGATGCCCCAAGCTGAGAACCAAGACCCAGACATCAAGG | |
| | CGCATGTGAACTCCCTGGGGGAGAACCTGAAGACCCTCA | |
| | GGCTGAGGCTACGGCGCTGTCATCGATTTCTTCCCTGTGA | |
| | AAACAAGAGCAAGGCCGTGGAGCAGGTGAAGAATGCCT | |
| | TTAATAAGCTCCAAGAGAAAGGCATCTACAAAGCCATGA | |
| | GTGAGTTTGACATCTTCATCAACTACATAGAAGCCTACAT | |
| | GACAATGAAGATACGAAACTGATAA | |
| PROTEIN | MGWSCIILFLVATATGVHSAHHHHHHHHGSSPGQGTQSENSC | SEQ ID |
| | THFPGNLPDMLRDLRDAFSRVKTFFQMKDQLDNLLLKESL | No: 10 |
| | LEDFKGYLGCQALSEMIQFYLEEVMPQAENQDPDIKAHVN | |
| | SLGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, N21E | Name: | 074 pExSyn2.0-huIL10-8X NHis-N21E |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 11 |
| | ACGGATCCAGCCCAGGCCAGGGCACCCAGTCTGAGAACA | |
| | GCTGCACCCACTTCCCAGGCAACCTGCCTGAGATGCTTC | |
| | GAGATCTCCGAGATGCCTTCAGCAGAGTGAAGACTTTCT | |
| | TTCAAATGAAGGATCAGCTGGACAACTTGTTGTTAAAGG | |
| | AGTCCTTGCTGGAGGACTTTAAGGGTTACCTGGGTTGCC | |
| | AAGCCTTGTCTGAGATGATCCAGTTTTACCTGGAGGAGG | |
| | TGATGCCCCAAGCTGAGAACCAAGACCCAGACATCAAGG | |
| | CGCATGTGAACTCCCTGGGGGAGAACCTGAAGACCCTCA | |
| | GGCTGAGGCTACGGCGCTGTCATCGATTTCTTCCCTGTGA | |
| | AAACAAGAGCAAGGCCGTGGAGCAGGTGAAGAATGCCT | |
| | TTAATAAGCTCCAAGAGAAAGGCATCTACAAAGCCATGA | |
| | GTGAGTTTGACATCTTCATCAACTACATAGAAGCCTACAT | |
| | GACAATGAAGATACGAAACTGATAA | |
| PROTEIN | MGWSCIILFLVATATGVHSAHHHHHHHHGSSPGQGTQSENSC | SEQ ID |
| | THFPGNLPEMLRDLRDAFSRVKTFFQMKDQLDNLLLKESLL | No: 12 |
| | EDFKGYLGCQALSEMIQFYLEEVMPQAENQDPDIKAHVNS | |
| | LGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, M22D | Name: | 092 pExSyn2.0-huIL 10-8X NHis-M22D |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 13 |
| | ACGGATCCAGCCCAGGCCAGGGCACCCAGTCTGAGAACA | |
| | GCTGCACCCACTTCCCAGGCAACCTGCCTAACGACCTTC | |
| | GAGATCTCCGAGATGCCTTCAGCAGAGTGAAGACTTTCT | |
| | TTCAAATGAAGGATCAGCTGGACAACTTGTTGTTAAAGG | |
| | AGTCCTTGCTGGAGGACTTTAAGGGTTACCTGGGTTGCC | |
| | AAGCCTTGTCTGAGATGATCCAGTTTTACCTGGAGGAGG | |
| | TGATGCCCCAAGCTGAGAACCAAGACCCAGACATCAAGG | |
| | CGCATGTGAACTCCCTGGGGGAGAACCTGAAGACCCTCA | |
| | GGCTGAGGCTACGGCGCTGTCATCGATTTCTTCCCTGTGA | |
| | AAACAAGAGCAAGGCCGTGGAGCAGGTGAAGAATGCCT | |
| | TTAATAAGCTCCAAGAGAAAGGCATCTACAAAGCCATGA | |
| | GTGAGTTTGACATCTTCATCAACTACATAGAAGCCTACAT | |
| | GACAATGAAGATACGAAACTGATAA | |
| PROTEIN | MGWSCIILFLVATATGVHSAHHHHHHHHGSSPGQGTQSENSC | SEQ ID |
| | THFPGNLPNDLRDLRDAFSRVKTFFQMKDQLDNLLLKESLL | No: 14 |
| | EDFKGYLGCQALSEMIQFYLEEVMPQAENQDPDIKAHVNS | |
| | LGENLKTLRLRLRRCHRFLPCENKSKAVEQVKNAFNKLQE | |
| | KGIYKAMSEFDIFINYIEAYMTMKIRN | |

| | | |
|---|---|---|
| | | Vector |
| Description: | Human IL-10, M22S | Name: |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 15 |
| | ACGGATCCAGCCCAGGCCAGGGCACCCAGTCTGAGAACA | |
| | GCTGCACCCACTTCCCAGGCAACCTGCCTAACTCGCTTC | |
| | GAGATCTCCGAGATGCCTTCAGCAGAGTGAAGACTTTCT | |
| | TTCAAATGAAGGATCAGCTGGACAACTTGTTGTTAAAGG | |
| | AGTCCTTGCTGGAGGACTTTAAGGGTTACCTGGGTTGCC | |
| | AAGCCTTGTCTGAGATGATCCAGTTTTACCTGGAGGAGG | |
| | TGATGCCCCAAGCTGAGAACCAAGACCCAGACATCAAGG | |
| | CGCATGTGAACTCCCTGGGGGAGAACCTGAAGACCCTCA | |
| | GGCTGAGGCTACGGCGCTGTCATCGATTTCTTCCCTGTGA | |
| | AAACAAGAGCAAGGCCGTGGAGCAGGTGAAGAATGCCT | |
| | TTAATAAGCTCCAAGAGAAAGGCATCTACAAAGCCATGA | |
| | GTGAGTTTGACATCTTCATCAACTACATAGAAGCCTACAT | |
| | GACAATGAAGATACGAAACTGATAA | |
| PROTEIN | MGWSCIILFLVATATGVHSAHHHHHHHHGSSPGQGTQSENSC | SEQ ID |
| | THFPGNLPNSLRDLRDAFSRVKTFFQMKDQLDNLLLKESLL | No: 16 |
| | EDFKGYLGCQALSEMIQFYLEEVMPQAENQDPDIKAHVNS | |
| | LGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, M22T | Name: | 095 pExSyn2.0-huIL10-8X NHis-M22T |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 17 |
| | ACGGATCCAGCCCAGGCCAGGGCACCCAGTCTGAGAACA | |
| | GCTGCACCCACTTCCCAGGCAACCTGCCTAACACGCTTC | |
| | GAGATCTCCGAGATGCCTTCAGCAGAGTGAAGACTTTCT | |
| | TTCAAATGAAGGATCAGCTGGACAACTTGTTGTTAAAGG | |
| | AGTCCTTGCTGGAGGACTTTAAGGGTTACCTGGGTTGCC | |
| | AAGCCTTGTCTGAGATGATCCAGTTTTACCTGGAGGAGG | |
| | TGATGCCCCAAGCTGAGAACCAAGACCCAGACATCAAGG | |
| | CGCATGTGAACTCCCTGGGGGAGAACCTGAAGACCCTCA | |
| | GGCTGAGGCTACGGCGCTGTCATCGATTTCTTCCCTGTGA | |
| | AAACAAGAGCAAGGCCGTGGAGCAGGTGAAGAATGCCT | |
| | TTAATAAGCTCCAAGAGAAAGGCATCTACAAAGCCATGA | |
| | GTGAGTTTGACATCTTCATCAACTACATAGAAGCCTACAT | |
| | GACAATGAAGATACGAAACTGATAA | |
| PROTEIN | MGWSCIILFLVATATGVHSAHHHHHHHHGSSPGQGTQSENSC | SEQ ID |
| | THFPGNLPNTLRDLRDAFSRVKTFFQMKDQLDNLLLKESLL | No: 18 |
| | EDFKGYLGCQALSEMIQFYLEEVMPQAENQDPDIKAHVNS | |
| | LGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, M22W | Name: | 107 pExSyn2.0-huIL10-8X NHis-M22W |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 19 |
| | ACGGATCCAGCCCAGGCCAGGGCACCCAGTCTGAGAACA | |
| | GCTGCACCCACTTCCCAGGCAACCTGCCTAACTGGCTTC | |
| | GAGATCTCCGAGATGCCTTCAGCAGAGTGAAGACTTTCT | |
| | TTCAAATGAAGGATCAGCTGGACAACTTGTTGTTAAAGG | |
| | AGTCCTTGCTGGAGGACTTTAAGGGTTACCTGGGTTGCC | |
| | AAGCCTTGTCTGAGATGATCCAGTTTTACCTGGAGGAGG | |
| | TGATGCCCCAAGCTGAGAACCAAGACCCAGACATCAAGG | |
| | CGCATGTGAACTCCCTGGGGGAGAACCTGAAGACCCTCA | |
| | GGCTGAGGCTACGGCGCTGTCATCGATTTCTTCCCTGTGA | |
| | AAACAAGAGCAAGGCCGTGGAGCAGGTGAAGAATGCCT | |
| | TTAATAAGCTCCAAGAGAAAGGCATCTACAAAGCCATGA | |
| | GTGAGTTTGACATCTTCATCAACTACATAGAAGCCTACAT | |
| | GACAATGAAGATACGAAACTGATAA | |
| PROTEIN | MGWSCIILFLVATATGVHSAHHHHHHHHGSSPGQGTQSENSC | SEQ ID |
| | THFPGNLPNWLRDLRDAFSRVKTFFQMKDQLDNLLLKESL | No: 20 |
| | LEDFKGYLGCQALSEMIQFYLEEVMPQAENQDPDIKAHVN | |
| | SLGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, E96C | Name: | 154 pExSyn2.0-huIL10-8X NHis-E96C |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 21 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCAGAGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACTCCCTGGGGTGCAACCTGAAGACCCTCAG | |
| | GCTGAGGCTACGGCGCTGTCATCGATTTCTTCCCTGTGAA | |
| | AACAAGAGCAAGGCCGTGGAGCAGGTGAAGAATGCCTT | |
| | TAATAAGCTCCAAGAGAAAGGCATCTACAAAGCCATGAG | |
| | TGAGTTTGACATCTTCATCAACTACATAGAAGCCTACATG | |
| | ACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSRVKTFFQMKDQLDNLLLKESL | No: 22 |
| | LEDFKGYLGCQALSEMIQFYLEEVMPQAENQDPDIKAHVN | |
| | SLGCNLKTLRLRLRRCHRFLPCENKSKAVEQVKNAFNKLQ | |
| | EKGIYKAMSEFDIFINYIEAYMTMKIRN | |

| | | | |
|---|---|---|---|
| | | Vector | |
| Description: | Human IL-10, E96F | Name: | 162 pExSyn2.0-huIL10-8X NHis-E96F |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 23 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCAGAGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACTCCCTGGGGTTCAACCTGAAGACCCTCAG | |
| | GCTGAGGCTACGGCGCTGTCATCGATTTCTTCCCTGTGAA | |
| | AACAAGAGCAAGGCCGTGGAGCAGGTGAAGAATGCCTT | |
| | TAATAAGCTCCAAGAGAAAGGCATCTACAAAGCCATGAG | |
| | TGAGTTTGACATCTTCATCAACTACATAGAAGCCTACATG | |
| | ACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSRVKTFFQMKDQLDNLLLKESL | No: 24 |
| | LEDFKGYLGCQALSEMIQFYLEEVMPQAENQDPDIKAHVN | |
| | SLGFNLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, E96Y | Name: | 163 pExSyn2.0-huIL10-8X NHis-E96Y |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 25 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCAGAGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACTCCCTGGGGTACAACCTGAAGACCCTCAG | |
| | GCTGAGGCTACGGCGCTGTCATCGATTTCTTCCCTGTGAA | |
| | AACAAGAGCAAGGCCGTGGAGCAGGTGAAGAATGCCTT | |
| | TAATAAGCTCCAAGAGAAAGGCATCTACAAAGCCATGAG | |
| | TGAGTTTGACATCTTCATCAACTACATAGAAGCCTACATG | |
| | ACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSRVKTFFQMKDQLDNLLLKESL | No: 26 |
| | LEDFKGYLGCQALSEMIQFYLEEVMPQAENQDPDIKAHVN | |
| | SLGYNLKTLRLRLRRCHRFLPCENKSKAVEQVKNAFNKLQ | |
| | EKGIYKAMSEFDIFINYIEAYMTMKIRN | |

| | | | |
|---|---|---|---|
| | | Vector | |
| Description: | Human IL-10, E96W | Name: | 164 pExSyn2.0-huIL 10-8X NHis-E96W |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 27 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCAGAGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACTCCCTGGGGTGGAACCTGAAGACCCTCAG | |
| | GCTGAGGCTACGGCGCTGTCATCGATTTCTTCCCTGTGAA | |
| | AACAAGAGCAAGGCCGTGGAGCAGGTGAAGAATGCCTT | |
| | TAATAAGCTCCAAGAGAAAGGCATCTACAAAGCCATGAG | |
| | TGAGTTTGACATCTTCATCAACTACATAGAAGCCTACATG | |
| | ACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSRVKTFFQMKDQLDNLLLKESL | No: 28 |
| | LEDFKGYLGCQALSEMIQFYLEEVMPQAENQDPDIKAHVN | |
| | SLGWNLKTLRLRLRRCHRFLPCENKSKAVEQVKNAFNKLQ | |
| | EKGIYKAMSEFDIFINYIEAYMTMKIRN | |

| | | | |
|---|---|---|---|
| | | Vector | |
| Description: | Human IL-10, T100C | Name: | 173 pExSyn2.0-huIL10-8X NHis-T100C |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 29 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCAGAGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACTCCCTGGGGGAGAACCTGAAGTGCCTCAG | |
| | GCTGAGGCTACGGCGCTGTCATCGATTTCTTCCCTGTGAA | |
| | AACAAGAGCAAGGCCGTGGAGCAGGTGAAGAATGCCTT | |
| | TAATAAGCTCCAAGAGAAAGGCATCTACAAAGCCATGAG | |
| | TGAGTTTGACATCTTCATCAACTACATAGAAGCCTACATG | |
| | ACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSRVKTFFQMKDQLDNLLLKESL | No: 30 |
| | LEDFKGYLGCQALSEMIQFYLEEVMPQAENQDPDIKAHVN | |
| | SLGENLKCLRLRLRRCHRFLPCENKSKAVEQVKNAFNKLQ | |
| | EKGIYKAMSEFDIFINYIEAYMTMKIRN | |

| | | | |
|---|---|---|---|
| | | Vector | |
| Description: | Human IL-10, H14D, T100L | Name: | 469 pExSyn2.0-huIL10-8X NHis-H14D, T100L |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 31 |
| | CCAGGGCACCCAGTCTGAGAACAGCTGCACCGACTTCCCAGGCAACCT | |
| | GCCTAACATGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGGAGAACCTGA | |
| | AGCTCCTCAGGCTGAGGCTACGGCGCTGTCATCGATTTCTTCCCTGTGA | |
| | AAACAAGAGCAAGGCCGTGGAGCAGGTGAAGAATGCCTTTAATAAGC | |
| | TCCAAGAGAAAGGCATCTACAAAGCCATGAGTGAGTTTGACATCTTCA | |
| | TCAACTACATAGAAGCCTACATGACAATGAAGATACGAAACTGATAA | |
| PROTEIN | MGWSCIILFLVATATGVHSAHHHHHHHHGSSPGQGTQSENSCTDFPGN | SEQ ID |
| | LPNMLRDLRDAFSRVKTFFQMKDQLDNLLLKESLLEDFKGYLGCQALSEM | No: 32 |
| | IQFYLEEVMPQAENQDPDIKAHVNSLGENLKLLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14D, N21K | Name: | 470 pExSyn2.0-huIL10-8X NHis-H14D, N21K |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 33 |
| | CCAGGGCACCCAGTCTGAGAACAGCTGCACCGACTTCCCAGGCAACCT | |
| | GCCTAAGATGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGGAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTDFPGN | SEQ ID |
| | LPKMLRDLRDAFSRVKTFFQMKDQLDNLLLKESLLEDFKGYLGCQALSEM | No: 34 |
| | IQFYLEEVMPQAENQDPDIKAHVNSLGENLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14D, M22D | Name: | 471 pExSyn2.0-huIL10-8X NHis-H14D, M22D |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 35 |
| | CCAGGGCACCCAGTCTGAGAACAGCTGCACCGACTTCCCAGGCAACCT | |
| | GCCTAACGACCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGGAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTDFPGN | SEQ ID |
| | LPNDLRDLRDAFSRVKTFFQMKDQLDNLLLKESLLEDFKGYLGCQALSEMI | No: 36 |
| | QFYLEEVMPQAENQDPDIKAHVNSLGENLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14D, M22S | Name: | 472 pExSyn2.0-huIL10-8X NHis-H14D, M22S |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 37 |
| | CCAGGGCACCCAGTCTGAGAACAGCTGCACCGACTTCCCAGGCAACCT | |
| | GCCTAACTCGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGGAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTDFPGN | SEQ ID |
| | LPNSLRDLRDAFSRVKTFFQMKDQLDNLLLKESLLEDFKGYLGCQALSEMI | No: 38 |
| | QFYLEEVMPQAENQDPDIKAHVNSLGENLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14D, M22A | Name: | 473 pExSyn2.0-huIL10-8X NHis-H14D, M22A |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 39 |
| | CCAGGGCACCCAGTCTGAGAACAGCTGCACCGACTTCCCAGGCAACCT | |
| | GCCTAACGCGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGGAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTDFPGN | SEQ ID |
| | LPNALRDLRDAFSRVKTFFQMKDQLDNLLLKESLLEDFKGYLGCQALSEMI | No: 40 |
| | QFYLEEVMPQAENQDPDIKAHVNSLGENLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14D, M22W | Name: | 474 pExSyn2.0-huIL10-8X NHis-H14D, M22W |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 41 |
| | CCAGGGCACCCAGTCTGAGAACAGCTGCACCGACTTCCCAGGCAACCT | |
| | GCCTAACTGGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGGAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTDFPGN | SEQ ID |
| | LPNWLRDLRDAFSRVKTFFQMKDQLDNLLLKESLLEDFKGYLGCQALSEM | No: 42 |
| | IQFYLEEVMPQAENQDPDIKAHVNSLGENLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14D, E74K | Name: | 475 pExSyn2.0-huIL10-8X NHis-H14D, E74K |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 43 |
| | CCAGGGCACCCAGTCTGAGAACAGCTGCACCGACTTCCCAGGCAACCT | |
| | GCCTAACATGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGAAGGAGGTGATGCCCCAAGCTGAGAACCA | |
| | AGACCCAGACATCAAGGCGCATGTGAACTCCCTGGGGGAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTDFPGN | SEQ ID |
| | LPNMLRDLRDAFSRVKTFFQMKDQLDNLLLKESLLEDFKGYLGCQALSEM | No: 44 |
| | IQFYLKEVMPQAENQDPDIKAHVNSLGENLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14D, E96K | Name: | 476 pExSyn2.0-huIL10-8X NHis-H14D, E96K |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 45 |
| | CCAGGGCACCCAGTCTGAGAACAGCTGCACCGACTTCCCAGGCAACCT | |
| | GCCTAACATGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGAAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTDFPGN | SEQ ID |
| | LPNMLRDLRDAFSRVKTFFQMKDQLDNLLLKESLLEDFKGYLGCQALSEM | No: 46 |
| | IQFYLEEVMPQAENQDPDIKAHVNSLGKNLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14G, T100L | Name: | 477 pExSyn2.0-huIL10-8X NHis-H14G, T100L |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 47 |
| | CCAGGGCACCCAGTCTGAGAACAGCTGCACCGGCTTCCCAGGCAACCT | |
| | GCCTAACATGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGGAGAACCTGA | |
| | AGCTCCTCAGGCTGAGGCTACGGCGCTGTCATCGATTTCTTCCCTGTGA | |
| | AAACAAGAGCAAGGCCGTGGAGCAGGTGAAGAATGCCTTTAATAAGC | |
| | TCCAAGAGAAAGGCATCTACAAAGCCATGAGTGAGTTTGACATCTTCA | |
| | TCAACTACATAGAAGCCTACATGACAATGAAGATACGAAACTGATAA | |
| PROTEIN | MGWSCIILFLVATATGVHSAHHHHHHHHGSSPGQGTQSENSCTGFPGN | SEQ ID |
| | LPNMLRDLRDAFSRVKTFFQMKDQLDNLLLKESLLEDFKGYLGCQALSEM | No: 48 |
| | IQFYLEEVMPQAENQDPDIKAHVNSLGENLKLLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14G, N21K | Name: | 478 pExSyn2.0-huIL10-8X NHis-H14G, N21K |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 49 |
| | CCAGGGCACCCAGTCTGAGAACAGCTGCACCGGCTTCCCAGGCAACCT | |
| | GCCTAAGATGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGGAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTGFPGN | SEQ ID |
| | LPKMLRDLRDAFSRVKTFFQMKDQLDNLLLKESLLEDFKGYLGCQALSEM | No: 50 |
| | IQFYLEEVMPQAENQDPDIKAHVNSLGENLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14G, M22D | Name: | 479 pExSyn2.0-huIL10-8X NHis-H14G, M22D |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 51 |
| | CCAGGGCACCCAGTCTGAGAACAGCTGCACCGGCTTCCCAGGCAACCT | |
| | GCCTAACGACCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGGAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTGFPGN | SEQ ID |
| | LPNDLRDLRDAFSRVKTFFQMKDQLDNLLLKESLLEDFKGYLGCQALSEMI | No: 52 |
| | QFYLEEVMPQAENQDPDIKAHVNSLGENLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14G, M22S | Name: | 480 pExSyn2.0-huIL10-8X NHis-H14G, M22S |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 53 |
| | CCAGGGCACCCAGTCTGAGAACAGCTGCACCGGCTTCCCAGGCAACCT | |
| | GCCTAACTCGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGGAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTGFPGN | SEQ ID |
| | LPNSLRDLRDAFSRVKTFFQMKDQLDNLLLKESLLEDFKGYLGCQALSEMI | No: 54 |
| | QFYLEEVMPQAENQDPDIKAHVNSLGENLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14G, M22A | Name: | 481 pExSyn2.0-huIL10-8X NHis-H14G, M22A |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 55 |
| | CCAGGGCACCCAGTCTGAGAACAGCTGCACCGGCTTCCCAGGCAACCT | |
| | GCCTAACGCGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGGAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTGFPGN | SEQ ID |
| | LPNALRDLRDAFSRVKTFFQMKDQLDNLLLKESLLEDFKGYLGCQALSEMI | No: 56 |
| | QFYLEEVMPQAENQDPDIKAHVNSLGENLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14G, M22W | Name: | 482 pExSyn2.0-huIL10-8X NHis-H14G, M22W |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 57 |
| | CCAGGGCACCCAGTCTGAGAACAGCTGCACCGGCTTCCCAGGCAACCT | |
| | GCCTAACTGGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGGAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTGFPGN | SEQ ID |
| | LPNWLRDLRDAFSRVKTFFQMKDQLDNLLLKESLLEDFKGYLGCQALSEM | No: 58 |
| | IQFYLEEVMPQAENQDPDIKAHVNSLGENLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14G, E74K | Name: | 483 pExSyn2.0-huIL10-8X NHis-H14G, E74K |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 59 |
| | CCAGGGCACCCAGTCTGAGAACAGCTGCACCGGCTTCCCAGGCAACCT | |
| | GCCTAACATGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGAAGGAGGTGATGCCCCAAGCTGAGAACCA | |
| | AGACCCAGACATCAAGGCGCATGTGAACTCCCTGGGGGAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTGFPGN | SEQ ID |
| | LPNMLRDLRDAFSRVKTFFQMKDQLDNLLLKESLLEDFKGYLGCQALSEM | No: 60 |
| | IQFYLKEVMPQAENQDPDIKAHVNSLGENLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, H14G, E96K | Name: | 484 pExSyn2.0-huIL10-8X NHis-H14G, E96K |

| | | |
|---|---|---|
| DNA | ATGGGATGGTCCTGCATCATCCTGTTTCTGGTGGCTACCGCCACCGGTG | SEQ ID |
| | TCCACTCTGCTCACCATCACCATCACCACCATCACGGATCCAGCCCAGG | No: 61 |
| | CCAGGGCACCCAGTCTGAGAACAGCTGCACCGGCTTCCCAGGCAACCT | |
| | GCCTAACATGCTTCGAGATCTCCGAGATGCCTTCAGCAGAGTGAAGAC | |
| | TTTCTTTCAAATGAAGGATCAGCTGGACAACTTGTTGTTAAAGGAGTC | |
| | CTTGCTGGAGGACTTTAAGGGTTACCTGGGTTGCCAAGCCTTGTCTGA | |
| | GATGATCCAGTTTTACCTGGAGGAGGTGATGCCCCAAGCTGAGAACCA | |
| | AGACCCAGACATCAAGGCGCATGTGAACTCCCTGGGGAAGAACCTGA | |
| | AGACCCTCAGGCTGAGGCTACGGCGCTGTCATCGATTTCTTCCCTGTG | |
| | AAAACAAGAGCAAGGCCGTGGAGCAGGTGAAGAATGCCTTTAATAAG | |
| | CTCCAAGAGAAAGGCATCTACAAAGCCATGAGTGAGTTTGACATCTTC | |
| | ATCAACTACATAGAAGCCTACATGACAATGAAGATACGAAACTGATAA | |
| PROTEIN | MGWSCIILFLVATATGVHSAHHHHHHHHGSSPGQGTQSENSCTGFPGN | SEQ ID |
| | LPNMLRDLRDAFSRVKTFFQMKDQLDNLLLKESLLEDFKGYLGCQALSEM | No: 62 |
| | IQFYLEEVMPQAENQDPDIKAHVNSLGKNLKTLRLRLRRCHRFLPCENKSK | |
| | AVEQVKNAFNKLQEKGIYKAMSEFDIFINYIEAYMTMKIRN | |

| | | | |
|---|---|---|---|
| | | Vector | |
| Description: | Human IL-10, N18R | Name: | 279 pExSyn2.0-huIL10-8X NHis-N18R |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 63 |
| | ACGGATCCAGCCCAGGCCAGGGCACCCAGTCTGAGAACA | |
| | GCTGCACCCACTTCCCAGGCCGCCTGCCTAACATGCTTC | |
| | GAGATCTCCGAGATGCCTTCAGCAGAGTGAAGACTTTCT | |
| | TTCAAATGAAGGATCAGCTGGACAACTTGTTGTTAAAGG | |
| | AGTCCTTGCTGGAGGACTTTAAGGGTTACCTGGGTTGCC | |
| | AAGCCTTGTCTGAGATGATCCAGTTTTACCTGGAGGAGG | |
| | TGATGCCCCAAGCTGAGAACCAAGACCCAGACATCAAGG | |
| | CGCATGTGAACTCCCTGGGGGAGAACCTGAAGACCCTCA | |
| | GGCTGAGGCTACGGCGCTGTCATCGATTTCTTCCCTGTGA | |
| | AAACAAGAGCAAGGCCGTGGAGCAGGTGAAGAATGCCT | |
| | TTAATAAGCTCCAAGAGAAAGGCATCTACAAAGCCATGA | |
| | GTGAGTTTGACATCTTCATCAACTACATAGAAGCCTACAT | |
| | GACAATGAAGATACGAAACTGATAA | |
| PROTEIN | MGWSCIILFLVATATGVHSAHHHHHHHHGSSPGQGTQSENSC | SEQ ID |
| | THFPGRLPNMLRDLRDAFSRVKTFFQMKDQLDNLLLKESL | No: 64 |
| | LEDFKGYLGCQALSEMIQFYLEEVMPQAENQDPDIKAHVN | |
| | SLGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, N18K | Name: | 281 pExSyn2.0-huIL10-8X NHis-N18K |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 65 |
| | ACGGATCCAGCCCAGGCCAGGGCACCCAGTCTGAGAACA | |
| | GCTGCACCCACTTCCCAGGCAAGCTGCCTAACATGCTTC | |
| | GAGATCTCCGAGATGCCTTCAGCAGAGTGAAGACTTTCT | |
| | TTCAAATGAAGGATCAGCTGGACAACTTGTTGTTAAAGG | |
| | AGTCCTTGCTGGAGGACTTTAAGGGTTACCTGGGTTGCC | |
| | AAGCCTTGTCTGAGATGATCCAGTTTTACCTGGAGGAGG | |
| | TGATGCCCCAAGCTGAGAACCAAGACCCAGACATCAAGG | |
| | CGCATGTGAACTCCCTGGGGGAGAACCTGAAGACCCTCA | |
| | GGCTGAGGCTACGGCGCTGTCATCGATTTCTTCCCTGTGA | |
| | AAACAAGAGCAAGGCCGTGGAGCAGGTGAAGAATGCCT | |
| | TTAATAAGCTCCAAGAGAAAGGCATCTACAAAGCCATGA | |
| | GTGAGTTTGACATCTTCATCAACTACATAGAAGCCTACAT | |
| | GACAATGAAGATACGAAACTGATAA | |
| PROTEIN | MGWSCIILFLVATATGVHSAHHHHHHHHGSSPGQGTQSENSC | SEQ ID |
| | THFPGKLPNMLRDLRDAFSRVKTFFQMKDQLDNLLLKESL | No: 66 |
| | LEDFKGYLGCQALSEMIQFYLEEVMPQAENQDPDIKAHVN | |
| | SLGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, R32N | Name: | 304 pExSyn2.0-huIL 10-8X NHis-R32N |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 67 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCAACGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACTCCCTGGGGGAGAACCTGAAGACCCTCAG | |
| | GCTGAGGCTACGGCGCTGTCATCGATTTCTTCCCTGTGAA | |
| | AACAAGAGCAAGGCCGTGGAGCAGGTGAAGAATGCCTT | |
| | TAATAAGCTCCAAGAGAAAGGCATCTACAAAGCCATGAG | |
| | TGAGTTTGACATCTTCATCAACTACATAGAAGCCTACATG | |
| | ACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSNVKTFFQMKDQLDNLLLKESL | No: 68 |
| | LEDFKGYLGCQALSEMIQFYLEEVMPQAENQDPDIKAHVN | |
| | SLGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, R32Q | Name: | 305 pExSyn2.0-huIL10-8X NHis-R32Q |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 69 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCCAAGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACTCCCTGGGGGAGAACCTGAAGACCCTCAG | |
| | GCTGAGGCTACGGCGCTGTCATCGATTTCTTCCCTGTGAA | |
| | AACAAGAGCAAGGCCGTGGAGCAGGTGAAGAATGCCTT | |
| | TAATAAGCTCCAAGAGAAAGGCATCTACAAAGCCATGAG | |
| | TGAGTTTGACATCTTCATCAACTACATAGAAGCCTACATG | |
| | ACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSQVKTFFQMKDQLDNLLLKESL | No: 70 |
| | LEDFKGYLGCQALSEMIQFYLEEVMPQAENQDPDIKAHVN | |
| | SLGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, R32G | Name: | 307 pExSyn2.0-huIL10-8X NHis-R32G |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 71 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCGGAGTGAAGACTTTCTT | |
| | TCAAATGAAGGATCAGCTGGACAACTTGTTGTTAAAGGA | |
| | GTCCTTGCTGGAGGACTTTAAGGGTTACCTGGGTTGCCA | |
| | AGCCTTGTCTGAGATGATCCAGTTTTACCTGGAGGAGGT | |
| | GATGCCCCAAGCTGAGAACCAAGACCCAGACATCAAGG | |
| | CGCATGTGAACTCCCTGGGGGAGAACCTGAAGACCCTCA | |
| | GGCTGAGGCTACGGCGCTGTCATCGATTTCTTCCCTGTGA | |
| | AAACAAGAGCAAGGCCGTGGAGCAGGTGAAGAATGCCT | |
| | TTAATAAGCTCCAAGAGAAAGGCATCTACAAAGCCATGA | |
| | GTGAGTTTGACATCTTCATCAACTACATAGAAGCCTACAT | |
| | GACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSGVKTFFQMKDQLDNLLLKESL | No: 72 |
| | LEDFKGYLGCQALSEMIQFYLEEVMPQAENQDPDIKAHVN | |
| | SLGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, R32C | Name: | 306 pExSyn2.0-huIL10-8X NHis-R32C |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 73 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCTGCGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACTCCCTGGGGGAGAACCTGAAGACCCTCAG | |
| | GCTGAGGCTACGGCGCTGTCATCGATTTCTTCCCTGTGAA | |
| | AACAAGAGCAAGGCCGTGGAGCAGGTGAAGAATGCCTT | |
| | TAATAAGCTCCAAGAGAAAGGCATCTACAAAGCCATGAG | |
| | TGAGTTTGACATCTTCATCAACTACATAGAAGCCTACATG | |
| | ACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSCVKTFFQMKDQLDNLLLKESL | No: 74 |
| | LEDFKGYLGCQALSEMIQFYLEEVMPQAENQDPDIKAHVN | |
| | SLGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, R32P | Name: | 308 pExSyn2.0-huIL10-8X NHis-R32P |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 75 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCCCAGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACTCCCTGGGGGAGAACCTGAAGACCCTCAG | |
| | GCTGAGGCTACGGCGCTGTCATCGATTTCTTCCCTGTGAA | |
| | AACAAGAGCAAGGCCGTGGAGCAGGTGAAGAATGCCTT | |
| | TAATAAGCTCCAAGAGAAAGGCATCTACAAAGCCATGAG | |
| | TGAGTTTGACATCTTCATCAACTACATAGAAGCCTACATG | |
| | ACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSPVKTFFQMKDQLDNLLLKESLL | No: 76 |
| | EDFKGYLGCQALSEMIQFYLEEVMPQAENQDPDIKAHVNS | |
| | LGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, R32F | Name: | 314 pExSyn2.0-huIL10-8X NHis-R32F |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 77 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCTTTGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACTCCCTGGGGGAGAACCTGAAGACCCTCAG | |
| | GCTGAGGCTACGGCGCTGTCATCGATTTCTTCCCTGTGAA | |
| | AACAAGAGCAAGGCCGTGGAGCAGGTGAAGAATGCCTT | |
| | TAATAAGCTCCAAGAGAAAGGCATCTACAAAGCCATGAG | |
| | TGAGTTTGACATCTTCATCAACTACATAGAAGCCTACATG | |
| | ACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSFVKTFFQMKDQLDNLLLKESLL | No: 78 |
| | EDFKGYLGCQALSEMIQFYLEEVMPQAENQDPDIKAHVNS | |
| | LGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, R32Y | Name: | 315 pExSyn2.0-huIL10-8X NHis-R32Y |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 79 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCTACGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACTCCCTGGGGGAGAACCTGAAGACCCTCAG | |
| | GCTGAGGCTACGGCGCTGTCATCGATTTCTTCCCTGTGAA | |
| | AACAAGAGCAAGGCCGTGGAGCAGGTGAAGAATGCCTT | |
| | TAATAAGCTCCAAGAGAAAGGCATCTACAAAGCCATGAG | |
| | TGAGTTTGACATCTTCATCAACTACATAGAAGCCTACATG | |
| | ACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSYVKTFFQMKDQLDNLLLKESL | No: 80 |
| | LEDFKGYLGCQALSEMIQFYLEEVMPQAENQDPDIKAHVN | |
| | SLGENLKTLRLRLRRCHRFLPCENKSKAVEQVKNAFNKLQE | |
| | KGIYKAMSEFDIFINYIEAYMTMKIRN | |

| | | | |
|---|---|---|---|
| | | Vector | |
| Description: | Human IL-10, S93G | Name: | 345 pExSyn2.0-huIL10-8X NHis-S93G |

| | | |
|---|---|---|
| DNA | ATGGGATGGTCCTGCATCATCCTGTTTCTGGTGGCTACCGCC | SEQ ID |
| | ACCGGTGTCCACTCTGCTCACCATCACCATCACCACCATC | No: 81 |
| | ACGGATCCAGCCCAGGCCAGGGCACCCAGTCTGAGAACA | |
| | GCTGCACCCACTTCCCAGGCAACCTGCCTAACATGCTTCG | |
| | AGATCTCCGAGATGCCTTCAGCAGAGTGAAGACTTTCTTT | |
| | CAAATGAAGGATCAGCTGGACAACTTGTTGTTAAAGGAG | |
| | TCCTTGCTGGAGGACTTTAAGGGTTACCTGGGTTGCCAA | |
| | GCCTTGTCTGAGATGATCCAGTTTTACCTGGAGGAGGTG | |
| | ATGCCCCAAGCTGAGAACCAAGACCCAGACATCAAGGC | |
| | GCATGTGAACGGCCTGGGGGAGAACCTGAAGACCCTCA | |
| | GGCTGAGGCTACGGCGCTGTCATCGATTTCTTCCCTGTGA | |
| | AAACAAGAGCAAGGCCGTGGAGCAGGTGAAGAATGCCT | |
| | TTAATAAGCTCCAAGAGAAAGGCATCTACAAAGCCATGA | |
| | GTGAGTTTGACATCTTCATCAACTACATAGAAGCCTACAT | |
| | GACAATGAAGATACGAAACTGATAA | |
| PROTEIN | MGWSCIILFLVATATGVHSAHHHHHHHHGSSPGQGTQSENSC | SEQ ID |
| | THFPGNLPNMLRDLRDAFSRVKTFFQMKDQLDNLLLKESL | No: 82 |
| | LEDFKGYLGCQALSEMIQFYLEEVMPQAENQDPDIKAHVN | |
| | GLGENLKTLRLRLRRCHRFLPCENKSKAVEQVKNAFNKLQ | |
| | EKGIYKAMSEFDIFINYIEAYMTMKIRN | |

Legend
Signal peptide italicized
Mutational changes from wild type in bold and underlined

TABLE 2
Wildtype hIL-10 and mutant hIL-10 Mature Protein Sequences

| | | | |
|---|---|---|---|
| | | Vector | |
| Description: | Human IL-10, Wildtype | Name: | pExSyn2.0-His-hIL10 WT (mp 43) |

| | | |
|---|---|---|
| PROTEIN | SPGQGTQSENSCTHFPGNLPNMLRDLRDAFSRVKTFFQMK | SEQ ID |
| | DQLDNLLLKESLLEDFKGYLGCQALSEMIQFYLEEVMPQAE | No: 88 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, N21C | Name: | 078 pExSyn2.0-huIL10-8X NHis-N21C |

| | | |
|---|---|---|
| PROTEIN | SPGQGTQSENSCTHFPGNLPCMLRDLRDAFSRVKTFFQMK | SEQ ID |
| | DQLDNLLLKESLLEDFKGYLGCQALSEMIQFYLEEVMPQAE | No: 89 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, N21D | Name: | 073 pExSyn2.0-huIL10-8X NHis-N21D |

| | | |
|---|---|---|
| PROTEIN | SPGQGTQSENSCTHFPGNLPDMLRDLRDAFSRVKTFFQMK | SEQ ID |
| | DQLDNLLLKESLLEDFKGYLGCQALSEMIQFYLEEVMPQAE | No: 90 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, N21E | Name: | 074 pExSyn2.0-huIL10-8X NHis-N21E |

| | | |
|---|---|---|
| PROTEIN | SPGQGTQSENSCTHFPGNLPEMLRDLRDAFSRVKTFFQMKD | SEQ ID |
| | QLDNLLLKESLLEDFKGYLGCQALSEMIQFYLEEVMPQAEN | No: 91 |
| | QDPDIKAHVNSLGENLKTLRLRLRRCHRFLPCENKSKAVEQ | |
| | VKNAFNKLQEKGIYKAMSEFDIFINYIEAYMTMKIRN | |

| | | | |
|---|---|---|---|
| | | Vector | |
| Description: | Human IL-10, M22D | Name: | 092 pExSyn2.0-huIL10-8X NHis-M22D |

| | | |
|---|---|---|
| PROTEIN | SPGQGTQSENSCTHFPGNLPNDLRDLRDAFSRVKTFFQMKD | SEQ ID |
| | QLDNLLLKESLLEDFKGYLGCQALSEMIQFYLEEVMPQAEN | No: 92 |
| | QDPDIKAHVNSLGENLKTLRLRLRRCHRFLPCENKSKAVEQ | |
| | VKNAFNKLQEKGIYKAMSEFDIFINYIEAYMTMKIRN | |

| | | | |
|---|---|---|---|
| | | Vector | |
| Description: | Human IL-10, M22S | Name: | 094 pExSyn2.0-huIL10-8X NHis-M22S |

| | | |
|---|---|---|
| PROTEIN | SPGQGTQSENSCTHFPGNLPNSLRDLRDAFSRVKTFFQMKD | SEQ ID |
| | QLDNLLLKESLLEDFKGYLGCQALSEMIQFYLEEVMPQAEN | No: 93 |
| | QDPDIKAHVNSLGENLKTLRLRLRRCHRFLPCENKSKAVEQ | |
| | VKNAFNKLQEKGIYKAMSEFDIFINYIEAYMTMKIRN | |

| | | | |
|---|---|---|---|
| | | Vector | |
| Description: | Human IL-10, M22T | Name: | 095 pExSyn2.0-huIL10-8X NHis-M22T |

| | | |
|---|---|---|
| PROTEIN | SPGQGTQSENSCTHFPGNLPNTLRDLRDAFSRVKTFFQMKD | SEQ ID |
| | QLDNLLLKESLLEDFKGYLGCQALSEMIQFYLEEVMPQAEN | No: 94 |
| | QDPDIKAHVNSLGENLKTLRLRLRRCHRFLPCENKSKAVEQ | |
| | VKNAFNKLQEKGIYKAMSEFDIFINYIEAYMTMKIRN | |

| | | | |
|---|---|---|---|
| | | Vector | |
| Description: | Human IL-10, M22W | Name: | 107 pExSyn2.0-huIL 10-8X NHis-M22W |

| | | |
|---|---|---|
| PROTEIN | SPGQGTQSENSCTHFPGNLPNWLRDLRDAFSRVKTFFQMK | SEQ ID |
| | DQLDNLLLKESLLEDFKGYLGCQALSEMIQFYLEEVMPQAE | No: 95 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, E96C | Name: | 154 pExSyn2.0-huIL10-8X NHis-E96C |

| | | |
|---|---|---|
| PROTEIN | SPGQGTQSENSCTHFPGNLPNMLRDLRDAFSRVKTFFQMK | SEQ ID |
| | DQLDNLLLKESLLEDFKGYLGCQALSEMIQFYLEEVMPQAE | No: 96 |
| | NQDPDIKAHVNSLGCNLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, E96F | Name: | 162 pExSyn2.0-huIL 10-8X NHis-E96F |

| | | |
|---|---|---|
| PROTEIN | SPGQGTQSENSCTHFPGNLPNMLRDLRDAFSRVKTFFQMK | SEQ ID |
| | DQLDNLLLKESLLEDFKGYLGCQALSEMIQFYLEEVMPQAE | No: 97 |
| | NQDPDIKAHVNSLGFNLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, E96Y | Name: | 163 pExSyn2.0-huIL 10-8X NHis-E96Y |

| | | |
|---|---|---|
| PROTEIN | SPGQGTQSENSCTHFPGNLPNMLRDLRDAFSRVKTFFQMK | SEQ ID |
| | DQLDNLLLKESLLEDFKGYLGCQALSEMIQFYLEEVMPQAE | No: 98 |
| | NQDPDIKAHVNSLGYNLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, E96W | Name: | 164 pExSyn2.0-huIL 10-8X NHis-E96W |

| | | |
|---|---|---|
| PROTEIN | SPGQGTQSENSCTHFPGNLPNMLRDLRDAFSRVKTFFQMK | SEQ ID |
| | DQLDNLLLKESLLEDFKGYLGCQALSEMIQFYLEEVMPQAE | No: 99 |
| | NQDPDIKAHVNSLGWNLKTLRLRLRRCHRFLPCENKSKAV | |
| | EQVKNAFNKLQEKGIYKAMSEFDIFINYIEAYMTMKIRN | |

| | | | |
|---|---|---|---|
| | | Vector | |
| Description: | Human IL-10, T100C | Name: | 173 pExSyn2.0-huIL10-8X NHis-T100C |

| | | |
|---|---|---|
| PROTEIN | SPGQGTQSENSCTHFPGNLPNMLRDLRDAFSRVKTFFQMK | SEQ ID |
| | DQLDNLLLKESLLEDFKGYLGCQALSEMIQFYLEEVMPQAE | No: 100 |
| | NQDPDIKAHVNSLGENLKCLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, H14D, T100L | Name: | 469 pExSyn2.0-huIL10-8X NHis-H14D, T100L |

| | | |
|---|---|---|
| PROTEIN | SPGQGTQSENSCTDFPGNLPNMLRDLRDAFSRVKTFFQMK | SEQ ID |
| | DQLDNLLLKESLLEDFKGYLGCQALSEMIQFYLEEVMPQAE | No: 101 |
| | NQDPDIKAHVNSLGENLKLLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, H14D, N21K | Name: | 470 pExSyn2.0-huIL 10-8X NHis-H14D, N21K |

| | | |
|---|---|---|
| PROTEIN | SPGQGTQSENSCTDFPGNLPKMLRDLRDAFSRVKTFFQMK | SEQ ID |
| | DQLDNLLLKESLLEDFKGYLGCQALSEMIQFYLEEVMPQAE | No: 102 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, H14D, M22D | Name: | 471 pExSyn2.0-huIL10-8X NHis-H14D, M22D |

| | | |
|---|---|---|
| PROTEIN | SPGQGTQSENSCTDFPGNLPNDLRDLRDAFSRVKTFFQMKD | SEQ ID |
| | QLDNLLLKESLLEDFKGYLGCQALSEMIQFYLEEVMPQAEN | No: 103 |
| | QDPDIKAHVNSLGENLKTLRLRLRRCHRFLPCENKSKAVEQ | |
| | VKNAFNKLQEKGIYKAMSEFDIFINYIEAYMTMKIRN | |

| | | | |
|---|---|---|---|
| | | Vector | |
| Description: | Human IL-10, H14D, M22S | Name: | 472 pExSyn2.0-huIL10-8X NHis-H14D, M22S |

| | | |
|---|---|---|
| PROTEIN | SPGQGTQSENSCTDFPGNLPNSLRDLRDAFSRVKTFFQMKD | SEQ ID |
| | QLDNLLLKESLLEDFKGYLGCQALSEMIQFYLEEVMPQAEN | No: 104 |
| | QDPDIKAHVNSLGENLKTLRLRLRRCHRFLPCENKSKAVEQ | |
| | VKNAFNKLQEKGIYKAMSEFDIFINYIEAYMTMKIRN | |

| | | | |
|---|---|---|---|
| | | Vector | |
| Description: | Human IL-10, H14D, M22A | Name: | 473 pExSyn2.0-huIL10-8X NHis-H14D, M22A |

| | | |
|---|---|---|
| PROTEIN | SPGQGTQSENSCTDFPGNLPNALRDLRDAFSRVKTFFQMKD | SEQ ID |
| | QLDNLLLKESLLEDFKGYLGCQALSEMIQFYLEEVMPQAEN | No: 105 |
| | QDPDIKAHVNSLGENLKTLRLRLRRCHRFLPCENKSKAVEQ | |
| | VKNAFNKLQEKGIYKAMSEFDIFINYIEAYMTMKIRN | |

| | | | |
|---|---|---|---|
| | | Vector | |
| Description: | Human IL-10, H14D, M22W | Name: | 474 pExSyn2.0-huIL10-8X NHis-H14D, M22W |

| | | |
|---|---|---|
| PROTEIN | SPGQGTQSENSCTDFPGNLPNWLRDLRDAFSRVKTFFQMK | SEQ ID |
| | DQLDNLLLKESLLEDFKGYLGCQALSEMIQFYLEEVMPQAE | No: 106 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, H14D, E74K | Name: | 475 pExSyn2.0-huIL10-8X NHis-H14D, E74K |

| | | |
|---|---|---|
| PROTEIN | SPGQGTQSENSCTDFPGNLPNMLRDLRDAFSRVKTFFQMK | SEQ ID |
| | DQLDNLLLKESLLEDFKGYLGCQALSEMIQFYLKEVMPQA | No: 107 |
| | ENQDPDIKAHVNSLGENLKTLRLRLRRCHRFLPCENKSKAV | |
| | EQVKNAFNKLQEKGIYKAMSEFDIFINYIEAYMTMKIRN | |

| | | | |
|---|---|---|---|
| | | Vector | |
| Description: | Human IL-10, H14D, E96K | Name: | 476 pExSyn2.0-huIL 10-8X NHis-H14D, E96K |

| | | |
|---|---|---|
| PROTEIN | SPGQGTQSENSCTDFPGNLPNMLRDLRDAFSRVKTFFQMK | SEQ ID |
| | DQLDNLLLKESLLEDFKGYLGCQALSEMIQFYLEEVMPQAE | No: 108 |
| | NQDPDIKAHVNSLGKNLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, H14G, T100L | Name: | 477 pExSyn2.0-huIL10-8X NHis-H14G, T100L |

| | | |
|---|---|---|
| PROTEIN | SPGQGTQSENSCTGFPGNLPNMLRDLRDAFSRVKTFFQMK | SEQ ID |
| | DQLDNLLLKESLLEDFKGYLGCQALSEMIQFYLEEVMPQAE | No: 109 |
| | NQDPDIKAHVNSLGENLKLLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, H14G, N21K | Name: | 478 pExSyn2.0-huIL 10-8X NHis-H14G, N21K |

| | | |
|---|---|---|
| PROTEIN | SPGQGTQSENSCTGFPGNLPKMLRDLRDAFSRVKTFFQMK | SEQ ID |
| | DQLDNLLLKESLLEDFKGYLGCQALSEMIQFYLEEVMPQAE | No: 110 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, H14G, M22D | Name: | 479 pExSyn2.0-huIL 10-8X NHis-H14G, M22D |

| | | |
|---|---|---|
| PROTEIN | SPGQGTQSENSCTGFPGNLPNDLRDLRDAFSRVKTFFQMKD | SEQ ID |
| | QLDNLLLKESLLEDFKGYLGCQALSEMIQFYLEEVMPQAEN | No: 111 |
| | QDPDIKAHVNSLGENLKTLRLRLRRCHRFLPCENKSKAVEQ | |
| | VKNAFNKLQEKGIYKAMSEFDIFINYIEAYMTMKIRN | |

| | | | |
|---|---|---|---|
| | | Vector | |
| Description: | Human IL-10, H14G, M22S | Name: | 480 pExSyn2.0-huIL10-8X NHis-H14G, M22S |

| | | |
|---|---|---|
| PROTEIN | SPGQGTQSENSCTGFPGNLPNSLRDLRDAFSRVKTFFQMKD | SEQ ID |
| | QLDNLLLKESLLEDFKGYLGCQALSEMIQFYLEEVMPQAEN | No: 112 |
| | QDPDIKAHVNSLGENLKTLRLRLRRCHRFLPCENKSKAVEQ | |
| | VKNAFNKLQEKGIYKAMSEFDIFINYIEAYMTMKIRN | |

| | | | |
|---|---|---|---|
| | | Vector | |
| Description: | Human IL-10, H14G, M22A | Name: | 481 pExSyn2.0-huIL10-8X NHis-H14G, M22A |

| | | |
|---|---|---|
| PROTEIN | SPGQGTQSENSCTGFPGNLPNALRDLRDAFSRVKTFFQMKD | SEQ ID |
| | QLDNLLLKESLLEDFKGYLGCQALSEMIQFYLEEVMPQAEN | No: 113 |
| | QDPDIKAHVNSLGENLKTLRLRLRRCHRFLPCENKSKAVEQ | |
| | VKNAFNKLQEKGIYKAMSEFDIFINYIEAYMTMKIRN | |

| | | | |
|---|---|---|---|
| | | Vector | |
| Description: | Human IL-10, H14G, M22W | Name: | 482 pExSyn2.0-huIL10-8X NHis-H14G, M22W |

| | | |
|---|---|---|
| PROTEIN | SPGQGTQSENSCTGFPGNLPNWLRDLRDAFSRVKTFFQMK | SEQ ID |
| | DQLDNLLLKESLLEDFKGYLGCQALSEMIQFYLEEVMPQAE | No: 114 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, H14G, E74K | Name: | 483 pExSyn2.0-huIL10-8X NHis-H14G, E74K |

| | | |
|---|---|---|
| PROTEIN | SPGQGTQSENSCTGFPGNLPNMLRDLRDAFSRVKTFFQMK | SEQ ID |
| | DQLDNLLLKESLLEDFKGYLGCQALSEMIQFYLKEVMPQA | No: 115 |
| | ENQDPDIKAHVNSLGENLKTLRLRLRRCHRFLPCENKSKAV | |
| | EQVKNAFNKLQEKGIYKAMSEFDIFINYIEAYMTMKIRN | |

| | | | |
|---|---|---|---|
| | Human IL-10, H14G, | Vector | |
| Description: | E96K | Name: | 484 pExSyn2.0-huIL 10-8X NHis-H14G, E96K |

| | | |
|---|---|---|
| PROTEIN | SPGQGTQSENSCTGFPGNLPNMLRDLRDAFSRVKTFFQMK | SEQ ID |
| | DQLDNLLLKESLLEDFKGYLGCQALSEMIQFYLEEVMPQAE | No: 116 |
| | NQDPDIKAHVNSLGKNLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, N18R | Name: | 279 pExSyn2.0-huIL 10-8X NHis-N18R |

| | | |
|---|---|---|
| PROTEIN | SPGQGTQSENSCTHFPGRLPNMLRDLRDAFSRVKTFFQMK | SEQ ID |
| | DQLDNLLLKESLLEDFKGYLGCQALSEMIQFYLEEVMPQAE | No: 117 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, N18K | Name: | 281 pExSyn2.0-huIL10-8X NHis-N18K |

| | | |
|---|---|---|
| PROTEIN | SPGQGTQSENSCTHFPGKLPNMLRDLRDAFSRVKTFFQMK | SEQ ID |
| | DQLDNLLLKESLLEDFKGYLGCQALSEMIQFYLEEVMPQAE | No: 118 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, R32N | Name: | 304 pExSyn2.0-huIL10-8X NHis-R32N |

| | | |
|---|---|---|
| PROTEIN | SPGQGTQSENSCTHFPGNLPNMLRDLRDAFSNVKTFFQMK | SEQ ID |
| | DQLDNLLLKESLLEDFKGYLGCQALSEMIQFYLEEVMPQAE | No: 119 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, R32Q | Name: | 305 pExSyn2.0-huIL 10-8X NHis-R32Q |

| | | |
|---|---|---|
| PROTEIN | SPGQGTQSENSCTHFPGNLPNMLRDLRDAFSQVKTFFQMK | SEQ ID |
| | DQLDNLLLKESLLEDFKGYLGCQALSEMIQFYLEEVMPQAE | No: 120 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, R32G | Name: | 307 pExSyn2.0-huIL10-8X NHis-R32G |

| | | |
|---|---|---|
| PROTEIN | SPGQGTQSENSCTHFPGNLPNMLRDLRDAFSGVKTFFQMK | SEQ ID |
| | DQLDNLLLKESLLEDFKGYLGCQALSEMIQFYLEEVMPQAE | No: 121 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, R32C | Name: | 306 pExSyn2.0-huIL10-8X NHis-R32C |

| | | |
|---|---|---|
| PROTEIN | SPGQGTQSENSCTHFPGNLPNMLRDLRDAFSCVKTFFQMK | SEQ ID |
| | DQLDNLLLKESLLEDFKGYLGCQALSEMIQFYLEEVMPQAE | No: 122 |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, R32P | Name: | 308 pExSyn2.0-huIL10-8X NHis-R32P |

| | | |
|---|---|---|
| PROTEIN | SPGQGTQSENSCTHFPGNLPNMLRDLRDAFSPVKTFFQMKD | SEQ ID |
| | QLDNLLLKESLLEDFKGYLGCQALSEMIQFYLEEVMPQAEN | No: 123 |
| | QDPDIKAHVNSLGENLKTLRLRLRRCHRFLPCENKSKAVEQ | |
| | VKNAFNKLQEKGIYKAMSEFDIFINYIEAYMTMKIRN | |

| | | | |
|---|---|---|---|
| | | Vector | |
| Description: | Human IL-10, R32F | Name: | 314 pExSyn2.0-huIL10-8X NHis-R32F |

| | | |
|---|---|---|
| PROTEIN | SPGQGTQSENSCTHFPGNLPNMLRDLRDAFSFVKTFFQMKD | SEQ ID |
| | QLDNLLLKESLLEDFKGYLGCQALSEMIQFYLEEVMPQAEN | No: 124 |
| | QDPDIKAHVNSLGENLKTLRLRLRRCHRFLPCENKSKAVEQ | |
| | VKNAFNKLQEKGIYKAMSEFDIFINYIEAYMTMKIRN | |

| | | | |
|---|---|---|---|
| | | Vector | |
| Description: | Human IL-10, R32Y | Name: | 315 pExSyn2.0-huIL10-8X NHis-R32Y |

| | | |
|---|---|---|
| PROTEIN | SPGQGTQSENSCTHFPGNLPNMLRDLRDAFSYVKTFFQMK | SEQ ID |
| | DQLDNLLLKESLLEDFKGYLGCQALSEMIQFYLEEVMPQAE | |
| | NQDPDIKAHVNSLGENLKTLRLRLRRCHRFLPCENKSKAVE | |
| | QVKNAFNKLQEKGIYKAMSEFDIFINYIEAYMTMKIRN | |

| | | | |
|---|---|---|---|
| | | Vector | |
| Description: | Human IL-10, S93G | Name: | 345 pExSyn2.0-huIL10-8X NHis-S93G |

| | | |
|---|---|---|
| PROTEIN | SPGQGTQSENSCTHFPGNLPNMLRDLRDAFSRVKTFFQMK | SEQ ID |
| | DQLDNLLLKESLLEDFKGYLGCQALSEMIQFYLEEVMPQAE | No: 126 |
| | NQDPDIKAHVNGLGENLKTLRLRLRRCHRFLPCENKSKAV | |
| | EQVKNAFNKLQEKGIYKAMSEFDIFINYIEAYMTMKIRN | |

Legend
Mutational changes from wild type in bold and underlined

What is claimed is:

1. A hIL10 monomer variant having at least 70% sequence identity (e.g., at least 70%, 75%, 80%, 85%, 90%, 91%, 92%, 93%, 94%, 95%, 96%, 97%, 98%, or 99% sequence identity) to SEQ ID NO:1 and comprising one or more amino acid substitutions at a position corresponding to residues H14, N18, N21, M22, D25, R32, S93, E96, and T100 of SEQ ID NO:1.

2. The hIL10 monomer variant of claim 1 comprising one or more amino substitutions at a position corresponding to amino acid residues selected from the group consisting of H14, N18, N21, M22, D25, R32, S93, E96, and T100 of SEQ ID NO:1, wherein:
the amino acid substitution at position H14 is selected from the group consisting of H14C, H14F, H14P, H14W and H14G;
the amino acid substitution at position N18 is selected from the group consisting of N18R and N18K;
the amino acid substitution at position N21 is selected from the group consisting of N21C, N21D and N21E;
the amino acid substitution at position M22 is selected from the group consisting of M22D, M22S, M22T, and M22W;
the amino acid substitution at position D25 is selected from the group consisting of D25P and D25Q;
the amino acid substitution at position R32 is selected from the group consisting of R32N, R32Q, R32G, R32C, R32P, R32F, and R32Y;
the amino acid substitution at position S93 is S93G;
the amino acid substitution at position E96 is selected from the group consisting of E96C, E96F, E96Y, and E96W; and
the amino acid substitution at position T100 is T100C.

3. The hIL10 monomer variant of claim 2 further comprising one or more amino substitutions selected from the group consisting of:
H14A, H14D, H14E, H14I, H14K, H14L, H14M, H14N, H14Q, H14R, H14S, H14T, H14Y, and H14V;
N18Y, N18F, N18A, N18D, N18E, N18L, N18V, N18S, N18T, N18I, N18V, N18M, and N18H;
N21A, N21R, N21Q, N21H, N21K, N21S, N21V, N21I, N21L, N21M, and N21T,
M22A, M22V, M22I, M22L, M22N, and M22Q;
R24E, R24D, R24N, R24Q, R24A, R24S, and R24T;
D25A, D25N, D25H, D25I, D25K, D25L, and D25V;
D28A, D28E, D28L, D28V, D28S, D28T, D28I, D28V, D28M, D28H, D28K, and D28R;
R32A, R32D, R32E, R32L, R32V, R32S, R32T, R32I, R32V, R32M, and R32H;
E74A, E74D, E74L, E74V, E74S, E74T, E74I, E74V, E74M, E74H, E74K, and E74R;
H90A, H90D, H90E, H90I, H90K, H90L, H90M, H90N, H90Q, H90R, H90S, H90T, H90Y, and H90V;
N92D, N92Q, N92E, N92H, N92K, N92S, N92V, N92I, N92L, N92M, N92T, and N92A;
S93E, S93A, S93R, S93N, S93D, S93Q, S93E, S93I, S93L, S93K, S93M, and S93V;
E96A, E96N, E96D, E96Q, E96H, E96K, and E96S;
T100D, T100V, T100E, T100A, T100R, T100N, T100Q, T100E, T100I, T100L, T100K, T100M, and T100S; or
R104A, R104W, R104Y, R104F, R104H, R104D, R104E, R104N, R104Q,
R104S, R104T, R104I, R104L, R104V, and R104M.

4. The hIL10 monomer variant of claim 2 further comprising an amino acid substitution at the position corresponding to N18 of SEQ ID NO:1 selected from the group consisting of Y, F, A, D, E, L, V, S, T, I, V, M, and H.

5. The hIL10 monomer variant of claim 2 further comprising an amino acid substitution at the position corresponding to N21 of SEQ ID NO:1 selected from the group consisting of A, R, Q, H, K, S, V, I, L, M, and T.

6. The hIL10 monomer variant of claim 2 further comprising an amino acid substitution at the position corresponding to M22 of SEQ ID NO:1 selected from the group consisting of A, V, I, L, N, and Q.

7. The hIL10 monomer variant of claim 2 further comprising an amino acid substitution at the position corresponding to R24 of SEQ ID NO:1 selected from the group consisting of E, D, N, Q, A, S, and T.

8. The hIL10 monomer variant of claim 2 further comprising an amino acid substitution at the position corresponding to D25 of SEQ ID NO:1 selected from the group consisting of A, N, H, I, K, L, and V.

9. The hIL10 monomer variant of claim 2 further comprising an amino acid substitution at the position corresponding to D28 of SEQ ID NO:1 selected from the group consisting of A, E, L, V, S, T, I, V, M, H, K, and R.

10. The hIL10 monomer variant of claim 2 further comprising an amino acid substitution at the position corresponding to R32 of SEQ ID NO:1 selected from the group consisting of A, D, E, L, V, S, T, I, V, M, and H.

11. The hIL10 monomer variant of claim 2 further comprising an amino acid substitution at the position corresponding to E74 of SEQ ID NO:1 selected from the group consisting of A, D, L, V, S, T, I, V, M, H, K, and R.

12. The hIL10 monomer variant of claim 2 further comprising an amino acid substitution at the position corresponding to H90 of SEQ ID NO:1 selected from the group consisting of A, D, E, I, K, L, M, N, Q, R, S, T, Y, and V.

13. The hIL10 monomer variant of claim 2 further comprising an amino s acid substitution at the position corresponding to N92 of SEQ ID NO:1 selected from the group consisting of D, Q, E, H, K, S, V, I, L, M, T, and A.

14. The hIL10 monomer variant of claim 2 further comprising an amino substitution at the position corresponding to S93 of SEQ ID NO:1 selected from the group consisting of E, A, R, N, D, Q, E, I, L, K, M, and V.

15. The hIL10 monomer variant of claim 2 further comprising an amino acid substitution at the position corresponding to E96 of SEQ ID NO:1 selected from the group consisting of A, N, D, Q, H, K, and S.

16. The hIL10 monomer variant of claim 2 further comprising an amino acid substitution at the position corresponding to T100 of SEQ ID NO:1 selected from the group consisting of D, V, E, A, R, N, Q, E, I, L, K, M, and S.

17. The hIL10 monomer variant of claim 2 further comprising an amino acid substitution at the position corresponding to R104 of SEQ ID NO:1 selected from the group consisting of A, W, Y, F, H, D, E, N, Q, S, T, I, L, V, and M.

18. The hIL10 monomer variant of claim 2 wherein the IL10 polypeptide further comprises an amino acid substitution selected from the group consisting of H14A, H14D, H14E, H14I, H14K, H14L, H14M, H14N, H14Q, H14R, H14S, H14T, H14Y, and H14V.

19. A hIL10 mutein comprising a hIL10 monomer variant of any one of claims 1-18 and a wild-type hIL10 monomer.

20. A hIL10 mutein comprising a first hIL10 monomer variant of any one of claims 1-18 and a second hIL10 monomer variant of any one of claims 1-18.

21. The hIL10 mutein of claim 20 wherein the first hIL10 monomer variant and the second hIL10 monomer have the same amino acid sequence.

22. The hIL10 mutein of claim 20 wherein the first hIL10 monomer variant and the second hIL10 monomer have different amino acid sequences.

23. An hIL10 mutein comprising an hIL10 monomer variant of any one of claims 1-18 wherein the hIL10 mutein possesses: (i) a pSTAT3 Emax in a myelocyte greater than 20% of the pSTAT3 Emax of IL10 in the myelocyte; and (b) a pSTAT3 Emax in a lymphocyte less than 70% but greater than 20% of the pSTAT3 Emax of IL10 in the lymphocyte.

24. The hIL10 mutein of claim 23 wherein the lymphocyte is a CD8+ T cell, a CD4+ T cell, a B cell or an NK cell.

25. The hIL10 mutein of claim 23 wherein the myelocyte cell is a neutrophil, eosinophil, mast cell, basophil or monocyte.

26. The hIL10 mutein of claim 23 wherein the monocyte is a dendritic cell or a macrophage.

27. The hIL10 mutein of claim 23 wherein the macrophage is a Kupffer cell.

28. An hIL10 mutein of any one of claims 19-22 comprising an hIL10 monomer variant of any one of claims 1-18 conjugated to one or more carrier molecules.

29. The hIL10 mutein of claim 28 wherein the carrier molecule is selected from a protein carrier molecule, Fc polypeptides and albumin polypeptides.

30. The hIL10 mutein of claim 28 wherein the carrier molecule is a water-soluble polymeric carrier.

31. The hIL10 mutein of claim 30 wherein the water-soluble polymeric carrier is polyethylene glycol (PEG).

32. The hIL10 mutein of claim 31 wherein the PEG is a linear or branched PEG having a molecular weight from about 5 kDA to about 80 kDA.

33. The hIL10 mutein of claim 31 wherein the PEG is conjugated to the N-terminus of the IL10 polypeptide.

34. The hIL10 mutein of any one of claims 28-33 wherein the carrier molecule is conjugated to at least one hIL10 monomer polypeptide via a linker.

35. A nucleic acid molecule comprising a nucleic acid sequence encoding an hIL10 monomer variant of any one of claims 1-18.

36. The nucleic acid molecule of claim 35, wherein the nucleic acid molecule further encodes a signal peptide.

37. The nucleic acid molecule of claim 35 or 36, wherein the nucleic acid molecule further encodes a linker.

38. The nucleic acid molecule of claims 35-37, wherein the nucleic acid sequence is operably linked to a heterologous nucleic acid sequence.

39. The nucleic acid molecule of claim 38 wherein the heterologous nucleic acid sequence is an expression control sequence.

40. An expression vector comprising a nucleic acid molecule of any one of claims 35-39.

41. A recombinantly modified cell comprising a nucleic acid molecule of any one of claims 35-39.

42. The cell of claim 41, wherein the cell is a prokaryotic cell.

43. The cell of claim 41, wherein the prokaryotic cell is a bacterial cell.

44. The cell of claim 41, wherein the cell is a eukaryotic cell.

45. The cell of claim 41, wherein the eukaryotic cell is a mammalian cell.

46. A cell culture comprising at least one cell of any one of claims 41-45, and a culture medium.

47. A method for producing a hIL10 mutein of any one of claims 19-23, the method comprising steps of:
a) providing one or more cells of any one of claims 41-45; and
b) culturing the one or more cells in a culture medium such that the cells produce the hIL10 monomer variant encoded by the nucleic acid molecule.

48. The method of claim 47, the method further comprising the step:
(c) isolating and/or purifying the hIL10 mutein.

49. A hIL10 mutein produced by the method of any one of claims 47-48.

50. A pharmaceutical composition comprising a pharmaceutically acceptable carrier and:
a) an hIL10 mutein comprising an hIL10 monomer variant of any one of claims 1-18;
b) an hIL10 mutein of any one of claims 19-34;
c) a nucleic acid of any one of claims 35-39; and/or
c) a cell of claim 41 or 45.

51. A method for modulating IL10-mediated signaling in a subject, the method comprising administering to the subject an effective amount of a pharmaceutical composition of claim 50.

52. The method of claim 51, wherein the IL10-mediated signaling comprises STAT3-mediated signaling.

53. The method of claim 52, wherein the STAT3-mediated signaling is determined by an assay selected from the group consisting of a gene expression assay, a phospho-flow signaling assay, and an enzyme-linked immunosorbent assay (ELISA).

54. The method of any one of claims 51-53, wherein the STAT3-mediated signaling in the subject is reduced by about 20% to about 100% compared to a reference level.

55. The method of any one of claims 51-54, wherein the administered composition results in a reduced capacity to induce expression of a pro-inflammatory gene selected from IFN-7, granzyme B, granzyme A, perforin, TNF-α, GM-CSF, and MIP1α in the subject.

56. A method for the treatment of a health condition in a subject in need thereof, the method comprising administering to the subject a composition comprising:
a) an hIL10 mutein comprising an hIL10 monomer variant of any one of claims 1-18;
b) an hIL10 mutein of any one of claims 19-34;
c) a nucleic acid of any one of claims 35-39; and/or
c) a cell of claim 41 or 45.

57. A kit for modulating IL10-mediated signaling in a subject, or treating a health condition in a subject in need thereof, the kit comprising:
a) an hIL10 mutein comprising an hIL10 monomer variant of any one of claims 1-18,
b) an hIL10 mutein of any one of claims 19-34
c) a nucleic acid of any one of claims 35-39; and/or
c) a cell of claim 41 or 45.
